(19) United States
(12) Patent Application Publication
(10) Pub. No.: US 2026/0096998 A1
(43) Pub. Date: Apr. 9, 2026

(54) NUCLEIC ACID COMPLEXES AND USES THEREOF

(71) Applicant: BioNTech SE, Mainz (DE)

(72) Inventors: Jorge Moreno Herrero, Mainz (DE); Heinrich Haas, Mainz (DE); Stephanie Erbar, Chiclana de la Frontera (ES); Theo Benjamin Stahl, Mainz (DE)

(21) Appl. No.: 19/114,817

(22) PCT Filed: Sep. 26, 2023
(86) PCT No.: PCT/EP2023/076608

§ 371 Date: Mar. 25, 2025

Related U.S. Application Data
(60) Provisional application No. 63/517,338, filed on Aug. 2, 2023.

(30) Foreign Application Priority Data
Sep. 26, 2022 (WO) PCT/IB22/59136

(51) Int. Cl. A61K 9/51; A61K 31/7105; A61K 47/22; A61K 47/26; A61K 47/64; A61K 47/69; B82Y 5/00; C12N 15/67; C12N 15/88

(52) U.S. Cl. CPC A61K 9/5123; A61K 31/7105; A61K 47/22; A61K 47/26; A61K 47/64; A61K 47/6935; C12N 15/67; C12N 15/88; B82Y 5/00

(57) ABSTRACT

The present disclosure provides complexes and methods of use. In some embodiments, a complex described herein is a complex comprising a cationic polymer, an anionic polymer, and a monomeric RNA molecule, wherein the cationic polymer and the monomeric RNA molecule form a core complex encapsulated by the anionic polymer. In some embodiments, a complex comprises a linear cationic polymer, an anionic polymer, and a monomeric RNA molecule, wherein the cationic polymer and the monomeric RNA molecule form a core complex encapsulated by the anionic polymer.

### CROSS-REFERENCE TO RELATED APPLICATIONS

The present application claims priority PCT App. No. PCT/IB22/59136, filed Sep. 26, 2022, and U.S. Provisional Application No. 63/517,338, filed Aug. 2, 2023, the entirety contents of each of which are hereby incorporated by reference in their entirety.

### BACKGROUND

Use of nucleic acids, e.g., RNA, has emerged as a means for delivering therapy in recent years. Nucleic acid therapies have shown particular success in the space of infectious disease vaccines, and have exhibited promise as cancer immunotherapies, therapeutic protein replacement therapies, and treatment of genetic diseases. Delivering nucleic acid therapy to a cellular target faces certain challenges, as RNA is unstable and prone to degradation by nucleases. See Wadhwa, et al., Pharmaceutics, 12 (2): 102 (2020).

### SUMMARY

There remains a need for delivery vehicles of nucleic acids, e.g., RNA, that are capable of withstanding the conditions in the body, while effectively and efficiently delivering the nucleic acid to a target cell, without requiring substantial amounts of the nucleic acid to achieve such effectiveness. Cationic polymers have been recognized as useful for developing such delivery vehicles, as reported in PCT App. Pub. No. WO 2021/001417, the entirety of which is incorporated herein by reference. It is desirable, however, to provide a delivery vehicle that improves functionality relative to previous formulations, such as by providing a delivery vehicle that exhibits improved stealth properties, improved or altered targeting of cells or tissues, includes tracking functionality (e.g., a moiety that can be imaged, such as a radionuclide, a fluorophore, or a chelating agent), and/or can beneficially respond to particular physiological conditions, such as pH, temperature, or redox potential.

The present disclosure provides, among other things, complexes for delivery nucleic acids, e.g., RNA, that exhibit certain improvements over previous complex formulations. In some embodiments, the present disclosure provides a complex comprising a cationic polymer, an anionic polymer, and RNA, wherein the cationic polymer and the RNA form a core complex encapsulated by the anionic polymer. Such complexes, that exhibit improved properties described above, can be used in applications not previously realized by prior formulations, such as administration by systemic, subcutaneous, or intranasal means. Moreover, in some embodiments, provided complexes allow for specific tuning of the charge on the surface, preferably from about −25 mV to about +25 mV, as well as exhibit particular stability when exposed to multiple freeze-thaw cycles while maintaining acceptable PDI and constant size distribution.

It has also surprisingly been discovered that particular cationic polymers, e.g. cationic polymers having a linear structure confer improved properties relative to those having a branched structure. For example, as illustrated in Example 7, it was surprisingly found that a complex comprising a linear cationic polymer conferred improved transfection in a variety of cell lines relative to a complex comprising a branched cationic polymer. Accordingly, in some embodiments, the present disclosure provides a complex comprising a linear cationic polymer, an anionic polymer, and RNA, wherein the linear cationic polymer and the RNA form a core complex encapsulated by the anionic polymer.

In some embodiments, the present disclosure provides a method of increasing or causing increased expression of RNA in a target in a subject comprising administering to the subject a complex described herein.

In some embodiments, the present disclosure provides a method of treating a disease, disorder, or condition in a subject comprising administering to the subject a complex described herein.

In some embodiments, the present disclosure provides a complex described herein, for use as a medicament.

In some embodiments, the present disclosure provides a complex described herein, for use in the treatment and/or prevention of a disease or disorder, wherein the disease or disorder is an infectious disease, cancer, a genetic disorder, an autoimmune disease, or a rare disease.

### BRIEF DESCRIPTION OF THE DRAWING

FIG. 1 is a bar graph illustrating the average hydrodynamic diameter of formulations comprising saRNA after incubation in 10% v/v human serum at 37° C. for 30 minutes.

FIGS. 2A-C illustrates physicochemical characterization of Alfa-tagged ternary modRNA PEI-PLXs with PGA(50)-b-pSar(50)-Alfa, PGA(50)-Alfa, PGA(100)-Alfa and PGA(50)-b-Ac-AEEA(14)-Alfa with Glu/N ratio from 0 to 3. FIG. 2A is a plot measuring particle size distribution (Zavg). FIG. 2B is a plot measuring polydispersity index (PDI). FIG. 2C is a plot measuring zeta potential.

FIGS. 3A-3D illustrate Agarose gel electrophoresis for ternary modRNA PEI-PLXs with (FIG. 3A) PGA(50)-b-pSar(50)-Alfa (Glu/N 0, 0.2, 0.4, 0.6, 0.8, 1, 1.5 followed by naked saRNA control); (FIG. 3B) PGA(50)-Alfa (Glu/N 0, 0.2, 0.4, 0.6, 0.8, 1, 1.5 and 3 followed by naked saRNA control); (FIG. 3C) PGA(100)-Alfa (Glu/N 0, 0.2, 0.4, 0.6 followed by naked saRNA control) and (FIG. 3D) PGA(50)b-Ac-AEEA(14)-Alfa (Glu/N 0, 0.2, 0.4, 0.6, 0.8, 1, 1.5 followed by naked saRNA control). Each well indicates a sample at increasing Glu/N ratio, followed by naked modRNA control.

FIGS. 4A-F illustrate frozen stability of ternary modRNA PEI-PLXs showing Zavg and PDI. FIG. 4A: PGA(50)-b-pSar(50)-Alfa; FIGS. 4B and 4C: PGA(50)-Alfa; FIG. 4D: PGA(100)-Alfa; and FIGS. 4E and 4F: PGA(50)-b-Ac-AEEA(14)-Alfa, respectively, at −20° C. and −80° C.

FIGS. 5A-C are a set of plots illustrating physicochemical characterization of alfa-tagged PGA(X) ternary PEI polyplexes (with X=50, 100), where FIG. 5A illustrates particle size distribution; FIG. 5B illustrates polydispersity Index (PDI) distribution; and FIG. 5C illustrates zeta potential distribution.

FIG. 6 illustrates Agarose gel electrophoresis for ternary PEI-PLXs with PGA(X)-Alfa with X=50, 100. Each well indicates a sample at increasing Glu/N ratio from 0, 0.2, 0.4, 0.6, 0.8, 1, 1.5 (PGA(50)-Alfa) and from 0, 0.2, 0.4, 0.6 (PGA(100)-Alfa) followed by naked saRNA control.

FIGS. 7A-D illustrate frozen stability (Zavg and PDI) of ternary saRNA PEI-PLXs with bar graphs FIGS. 7A and 7B reporting PGA(50)-Alfa, and bar graphs FIGS. 7C and 7D reporting PGA(100)-Alfa at −20° C. and −80° C.

FIGS. 8A-C is a series of plots illustrating physicochemical characterization of saRNA polyplexes coated with PEI-PLXs with PGA(50)-b-pSar(50)-Alfa and PGA(50)-b-pAEEA(14)-Alfa., where FIG. 8A illustrates particle size distribution; FIG. 8B illustrates polydispersity index (PDI) distribution; FIG. 8C illustrates zeta potential distribution.

FIGS. 9A-9B illustrates Agarose gel electrophoresis for ternary PEI-PLXs with FIG. 9A illustrating PGA(50)-b-pSar(50)-Alfa; and FIG. 9B illustrating PGA(50)-b-pAEEA(14)-Alfa (Beach well represents increasing Glu/N ratios from 0, 0.2, 0.4, 0.6, 0.8, 1 (PGA(50)-Alfa) followed by naked saRNA control.

FIGS. 10A-D illustrates frozen stability (Z-avg and PDI) of ternary saRNA PEI-PLXs with FIGS. 10A and 10B illustrating PGA(50)-b-pSar(50)-Alfa; and FIGS. 10C and 10D illustrating PGA(50)-b-pAEEA(14)-Alfa at −20° C. and −80° C.

FIG. 11 illustrates particle size and PDI of functionalized polyplexes with N/P=15; Core particle. Size and PDI measurement were conducted on a DynaPro Plate Reader III (Wyatt Technology) at a concentration of 5 ng/μL (5 μL of sample was mixed with 95 μL of water) at 23° C. Before measurement, samples were mixed in well by pipetting. For each well, 10 acquisitions with an acquisition time of 5 s was used.

FIG. 12 illustrates Zeta potential of functionalized polyplexes. Zeta potential measurement was conducted using a Malvern Zetasizer Ultra Nano Series Instrument cuvette with a DTS1070 cuvette at a concentration of 0.002 μg/μL (16 μL of sample mixed with 784 μL of 10 mM HEPES (pH=6)) at 23° C. Each sample was measured in 12 runs (attenuator set to 11).

FIG. 13 illustrates Agarose gel electrophoresis analysis of functionalized polyplexes. Agarose gels were cast at 1%, using pH 7 TAE buffer and Gel-Red in the recommended dilution. For all samples, 10 μL (total nucleic acid (NA) concentration=0.1 μg/L) were loaded on the gel, resulting in a total NA mass of 1 μg in each well. Two references were pipetted (0.1 μg, 0.5 μg) for each NA. Of 6-fold loading dye, 2 μL were added to all samples and reference. Separation was conducted at 80 V and 500 mA for 40 minutes. The image was taken with the BioRad ChemiDOC imaging device (auto optimal exposure time).

FIGS. 14A-B illustrate PBMC transfection studies with functionalized polyplexes. 10 μl of the respective nanoparticle formulations (total cargo concentration=0.1 μg/μL, c (Thy1.1)=0.025 g/L) were prediluted in 50 μl X-Vivo15 in an ultra-low adhesion 96 well plate. 0.3e6 thawed human PBMCs were diluted in 50 μl human serum from male AB clotted whole blood and added to the nanoparticle dilution. After 30 min of incubation (37° C., 5% CO₂), 100 μl of X-Vivo15 with human IL-2 [200 U/ml] were added per well. Cells were cultivated (37° C., 5% CO2) for an additional 96 h. FIG. 14A shows alive cell counts of all cells (total alive; right y-axis) and of cell subpopulations (CD4+, CD8+, CD19+ and CD14+; left y-axis) analyzed via flow cytometry. FIG. 14B shows cell type-specific RNA-transfection (Thy1.1-RNA expression), analyzed via flow cytometry. Depicted are the percentages of Thy1.1-expressing cells out of all single and alive cells.

FIG. 15 illustrates transfection studies with functionalized polyplexes in Jurkat cells. 5 μl of the respective nanoparticle formulation were prediluted in 50 μl RPMI+10% FBS and 1% Pen/Strep in an ultra-low adhesion 96 well plate. 0.5e6 Jurkat cells were diluted in in 50 μl RPMI+10% FBS and 1% Pen/Strep and added to the nanoparticle dilution. After 30 min of incubation (37° C., 5% CO₂) 10 μl of Jurkat cells and nanoparticle solution were transferred into new wells and 190 μl RPMI+10% FBS and 1% Pen/Strep were added. Cells were cultivated (37° C., 5% CO₂) for an additional 96 h. FIG. 15 shows flow cytometry-based analysis of alive cell counts (Alive; right y-axis) and transfection, depicted as percentage of Thy1.1-RNA and Venus-DNA expressing cells out of all single and alive cells (Thy1.1/Venus; left y-axis).

FIGS. 16A-B illustrate physicochemical characterization of binary polyplexes prepared with linear (L-PEI) and branched (b-PEI) PEI and modRNA and saRNA. FIG. 16A illustrates particle size distribution and monomeric RNA. FIG. 16B is an image of agarose gel electrophoresis (each well indicates a sample, followed by naked modRNA or saRNA control).

FIGS. 17A-B illustrate frozen stability of binary polyplexes prepared with linear (L-PEI) and branched (b-PEI); FIG. 17A reports complexes with modRNA and FIG. 17B reports complexes with saRNA at −20° C. and −80° C.

FIGS. 18A-C illustrate in vitro transfection activity of modRNA binary polyplexes prepared with linear (L-PEI) and branched (b-PEI) PEI in HepG2 (FIG. 18A), RAW (FIG. 18B) and C2C12 (FIG. 18C) cell lines.

FIGS. 19A-C illustrate in vitro cell viability of modRNA binary polyplexes prepared with linear (L-PEI) and branched (b-PEI) PEI in HepG2 (FIG. 19A), RAW (FIG. 19B) and C2C12 (FIG. 19C) cell lines.

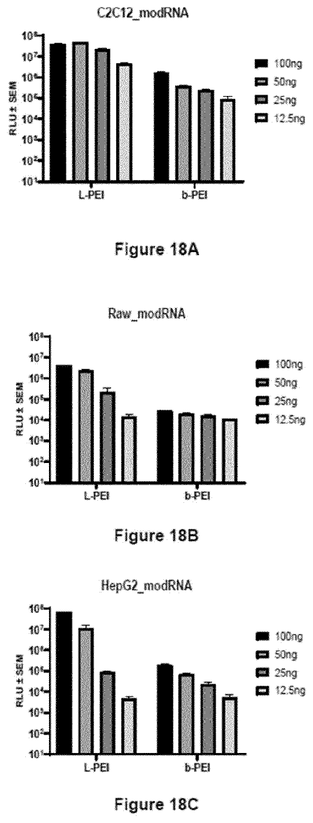

FIGS. 20A-C illustrate in vitro transfection activity of saRNA binary polyplexes prepared with linear (L-PEI) and branched (b-PEI) PEI in HepG2 (FIG. 20A), RAW (FIG. 20B) and C2C12 (FIG. 20C) cell lines.

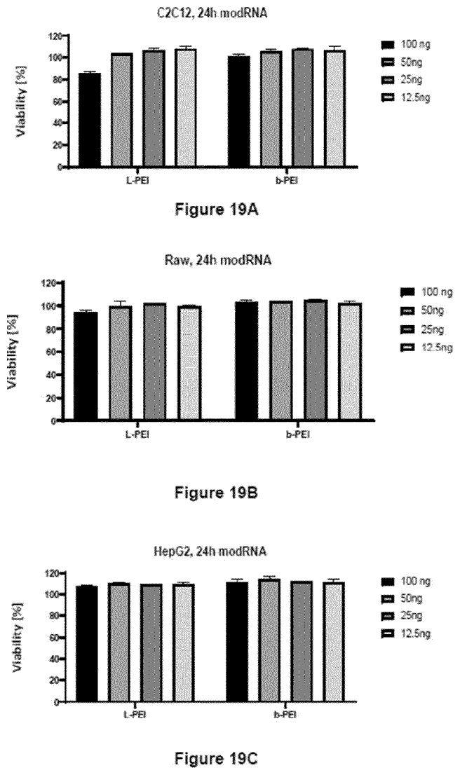

FIGS. 21A-C illustrate in vitro cell viability of saRNA binary polyplexes prepared with linear (L-PEI) and branched (b-PEI) PEI in HepG2 (FIG. 21A), RAW (FIG. 21B) and C2C12 (FIG. 21C) cell lines.

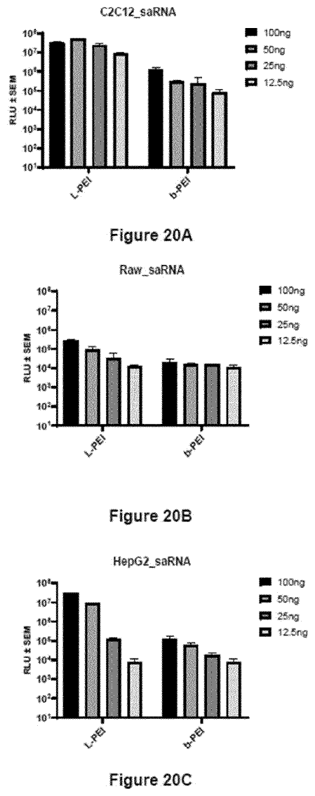

FIG. 22 illustrates physicochemical characterization for modRNA ternary PEI-PLXs with PGA(X) with X=20, 50. The top graph illustrates particle size distribution, the middle graph illustrates polydispersity index, and the bottom graph illustrates Zeta potential.

FIG. 23 illustrates agarose gel electrophoresis for ternary PEI-PLXs with PGA(X) with X=20, 50. Each well indicates a sample at increasing Glu/N ratio from 0, 0.2, 0.4, 0.8, 1, 1.5 and 3, followed by naked modRNA control for PGA(20) and Glu/N ratio from 0, 0.2, 0.4, 0.8, 1 and 1.5, followed by naked modRNA control for PGA(50).

FIG. 24 illustrates serum stability of ternary PEI-PLXs with PGA(X) with X=20, 50. For each set of ternary complexes, upper lane corresponds to control samples without serum incubation and bottom lane corresponds to samples that undergo serum incubation. Each well indicates a sample at increasing Glu/N ratio from 0, 0.2, 0.4, 0.8, 1, 1.5 and 3, followed by naked modRNA control for PGA(20) and Glu/N ratio from 0, 0.2, 0.4, 0.8, followed by naked modRNA control for PGA(50).

FIG. 25 illustrates frozen stability of ternary modRNA PEI-PLXs with PGA(20) and PGA(50), respectively, at −20° C. and −80° C.

FIG. 26 illustrates physicochemical characterization for ternary PEI-PLXs with PAsp(50). The top graph illustrates particle size distribution, the middle graph illustrates polydispersity index, and the bottom graph illustrates Zeta potential.

FIG. 27 illustrates agarose gel electrophoresis for ternary PEI-PLXs with PAsp(50). Each well indicates a sample at increasing Asp/N ratio from 0, 0.2, 0.4, 0.6, 0.8, 1, 1.5 and 3, followed by naked modRNA control.

FIG. 28 illustrates physicochemical characterization of ternary PEI-PLXs with PGA(50)-b-pSar(X) with X=10, 25, 50. The top graph illustrates particle size distribution, the middle graph illustrates polydispersity index, and the bottom graph illustrates Zeta potential.

FIG. 29 is an image of agarose gel electrophoresis for ternary PEI-PLXs with PGA(50)-b-pSar(X) with X=10, 25 and 50. Each well indicates a sample at increasing Glu/N ratio from 0, 0.2, 0.4, 0.6, followed by naked modRNA control.

FIG. 30 illustrates serum stability of ternary PEI-PLXs prepared with PGA(50)-b-pSar(50) at Glu/N 0.6. Upper lane corresponds to control samples without serum incubation and bottom lane corresponds to samples that undergo serum incubation. Wells order represent increasing incubation times, 30 min, 1 h, 2 h followed by naked modRNA control.

FIG. 31 illustrates cell viability of modRNA binary and ternary PLXs with PGA(50)-b-pSar(50) at Glu/N 0.6 in HepG2, RAW and C2C12 cell lines.

FIG. 32 illustrates in vitro transfection activity of modRNA binary and ternary PLXs with PGA(50)-b-pSar(50) at Glu/N 0.6 in HepG2, RAW and C2C12 cell lines.

FIG. 33 illustrates physicochemical characterization of Alfa tagged ternary modRNA PEI-PLXs with PGA(50)-b-pSar(50)-Alfa, PGA(50)-Alfa, PGA(100)-Alfa and PGA(50)-b-Ac-AEEA(14)-Alfa with Glu/N ratio from 0 to 3. The top graph illustrates particle size distribution, the middle graph illustrates polydispersity index, and the bottom graph illustrates Zeta potential.

FIG. 34 is an image of agarose gel electrophoresis for ternary modRNA PEI-PLXs with A) PGA(50)-b-pSar(50)-Alfa (Glu/N 0, 0.2 0.4, 0.6, 0.8, 1, 1.5)-x), B) PGA(50)-Alfa (Glu/N 0, 0.2, 0.4, 1, 1.5, 3), C) PGA(100)-Alfa (Glu/N 0, 0.2, 0.4) and D) PGA(50)b-Ac-AEEA(14)-Alfa (Glu/N 0, 0.2 0.4, 0.6, 0.8, 1, 1.5). Each well indicates a sample at increasing Glu/N ratio, followed by naked modRNA control.

FIG. 35 illustrates frozen stability of ternary modRNA PEI-PLXs with PGA(50)-b-pSar(50)-Alfa, PGA(50)-Alfa, PGA(100)-Alfa and PGA(50)-b-Ac-AEEA(14)-Alfa respectively, at −20° C. and −80° C.

FIG. 36 illustrates physicochemical characterization for saRNA ternary PEI-PLXs with PGA(X) with X=10, 20, 50. The top graph illustrates particle size distribution, the middle graph illustrates polydispersity index, and the bottom graph illustrates Zeta potential.

FIG. 37 is an image of agarose gel electrophoresis for ternary PEI-PLXs with PGA(X) with X=10, 20, 50. Each well indicates a sample at increasing Glu/N ratio from 0, 0.2, 0.4, 1.5 and 3, followed by naked saRNA control for PGA(10), Glu/N ratio from 0, 0.2, 0.4, 0.6, 0.8, 1, 1.5 and 3, followed by naked saRNA control for PGA(20), Glu/N ratio from 0, 0.2, 0.4, 0.8, 1, followed by naked saRNA control, for PGA(50).

FIG. 38 illustrates frozen stability of ternary saRNA PEI-PLXs at some of the selected Glu/N ratios, with A) PGA(10), B) PGA(20), C) PGA(50), respectively, at −20° C. and −80° C.

FIG. 39 illustrates physicochemical characterization for saRNA ternary PEI-PLXs with PGA(50)-b-pSar(X) with X=10, 25, 50. The top graph illustrates particle size distribution, the middle graph illustrates polydispersity index, and the bottom graph illustrates Zeta potential.

FIG. 40 is an image of agarose gel electrophoresis for ternary PEI-PLXs with PGA(50)-b-pSar(X) with X=10, 25 and 50. Each well indicates a sample at increasing Glu/N ratio from 0, 0.2, 0.4, 0.6, followed by naked saRNA control.

FIG. 41 illustrates frozen stability of ternary saRNA PEI-PLXs with A) PGA(50)-b-pSar(10), B) PGA(50)-b-pSar(25), PGA(50)-b-pSar(50) at −80° C.

FIG. 42 illustrates physicochemical characterization of alfa-tagged PGA(X) ternary PEI polyplexes (with X=50, 100). The top graph illustrates particle size distribution, the middle graph illustrates polydispersity index, and the bottom graph illustrates Zeta potential.

FIG. 43 is an image of agarose gel electrophoresis for ternary PEI-PLXs with PGA(X)-Alfa with X=50, 100. Each well indicates a sample at increasing Glu/N ratio from 0, 0.2, 0.4, 0.6, 0.8, 1, 1.5 followed by naked saRNA control for PGA(50)-Alfa, and Glu/N ratio from 0, 0.2, 0.4, 0.6 followed by naked saRNA control for PGA(100)-Alfa.

FIG. 44 illustrates frozen stability of ternary saRNA PEI-PLXs with PGA(50)-Alfa and PGA(100)-Alfa at −20° C. and −80° C.

FIG. 45 illustrates physicochemical characterization of saRNA polyplexes coated with PEI-PLXs with PGA(50)-b-pSar(50)-Alfa and PGA(50)-b-pAEEA(14)-Alfa. The top graph illustrates particle size distribution, the middle graph illustrates polydispersity index, and the bottom graph illustrates Zeta potential.

FIG. 46 is an image of agarose gel electrophoresis for ternary PEI-PLXs with PGA(50)-b-pSar(50)-Alfa and PGA(50)-b-pAEEA(14)-Alfa. Each well represents increasing Glu/N ratios from 0, 0.2, 0.4, 0.6, 0.8, 1, followed by naked saRNA control).

FIG. 47 illustrates frozen stability of ternary saRNA PEI-PLXs with PGA(50)-b-pSar(50)-Alfa (A) and PGA(50)-b-pAEEA(14)-Alfa (B) at −20° C. and −80° C.

FIG. 48 illustrates physicochemical characterization for saRNA ternary PGA(50)-b-Ac-AEEA(X) with X=4, 8 and 14. The top graph illustrates particle size distribution, the middle graph illustrates polydispersity index, and the bottom graph illustrates Zeta potential.

FIG. 49 is an image of agarose gel electrophoresis for ternary PEI-PLXs with PGA(50)-b-Ac-AEEA(X) with X=4, 8 and 14. Each well indicates increasing Glu/N ratio from 0, 0.2, 0.4, 0.6, 0.8, 1, followed by naked saRNA control for PGA(50)-b-pAEEA(4) and PGA(50)-b-pAEEA(8); and Glu/N ratio from 0, 0.2, 0.4, 0.6, 0.8, 1, 1.5 followed by naked saRNA control for PGA(50)-b-pAEEA(14).

FIG. 50 illustrates frozen stability of ternary saRNA PEI-PLXs with PGA(50)-b-Ac-AEEA(X) with X=4, 8 and 14 at −20° C. and −80° C.

### DETAILED DESCRIPTION OF CERTAIN EMBODIMENTS

The present disclosure provides, among other things, complexes useful for delivery of a nucleic acid, e.g., RNA, and uses of such complexes. In some embodiments, such complexes exhibit improved properties over previous formulations, including by one or more of: improved stealth properties, improved or altered targeting of cells or tissues, includes tracking functionality (e.g., a moiety that can be imaged, such as a radionuclide), and/or can beneficially respond to particular physiological conditions, such as pH, temperature, or redox potential. Moreover, it was also discovered that inclusion of a linear cationic polymer in complexes reported herein conferred improved properties even over complexes comprising a branched cationic polymer. For example, as illustrated in Example 7, use of a linear cationic polymer provided improved transfection efficiency (e.g., two to three log improvement) of target cells at a variety of concentrations relative to a branched polymer.

In some embodiments, complexes described herein are useful for the treatment of a variety of diseases. In some embodiments, such complexes can be administered via systemic, intravenous, or intranasal means. Moreover, in some embodiments, provided complexes allow for specific tuning of the charge on the surface, preferably from about −25 mV to about +25 mV, as well as exhibit particular stability when exposed to multiple freeze-thaw cycles while maintaining acceptable PDI and constant size distribution.

### Definitions

Complexes of this disclosure include those described generally above and are further illustrated by the classes, subclasses, and species disclosed herein. As used herein, the following definitions shall apply unless otherwise indicated. For purposes of this disclosure, the chemical elements are identified in accordance with the Periodic Table of Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Unless otherwise stated, structures depicted herein are meant to include all stereoisomeric (e.g., enantiomeric or diastereomeric) forms of the structure, as well as all geometric or conformational isomeric forms of the structure. For example, the R and S configurations of each stereocenter are contemplated as part of the disclosure. Therefore, single stereochemical isomers, as well as enantiomeric, diastereomeric, and geometric (or conformational) mixtures of provided compounds are within the scope of the disclosure. Unless otherwise stated, all tautomeric forms of provided compounds are within the scope of the disclosure.

Unless otherwise indicated, structures depicted herein are meant to include compounds that differ only in the presence of one or more isotopically enriched atoms. For example, compounds having the present structures including replacement of hydrogen by deuterium or tritium, or replacement of a carbon by ¹³C- or ¹⁴C-enriched carbon are within the scope of this disclosure.

About or approximately: As used herein, the term “approximately” or “about,” as applied to one or more values of interest, refers to a value that is similar to a stated reference value. In general, those skilled in the art, familiar within the context, will appreciate the relevant degree of variance encompassed by “about” or “approximately” in that context. For example, in some embodiments, the term “approximately” or “about” may encompass a range of values that are within (i.e., ±) 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to the administration of a composition to a subject to achieve delivery of an agent that is, or is included in, a composition to a target site or a site to be treated. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may be parenteral. In some embodiments, administration may be oral. In some particular embodiments, administration may be intravenous. In some particular embodiments, administration may be subcutaneous. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, administration may comprise a prime-and-boost protocol. A prime-and-boost protocol can include administration of a first dose of a pharmaceutical composition (e.g., an immunogenic composition, e.g., a vaccine) followed by, after an interval of time, administration of a second or subsequent dose of a pharmaceutical composition (e.g., an immunogenic composition, e.g., a vaccine). In the case of an immunogenic composition, a prime-and-boost protocol can result in an increased immune response in a patient.

Aliphatic: The term “aliphatic” refers to a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “cycloaliphatic”), that has a single point or more than one points of attachment to the rest of the molecule. Unless otherwise specified, aliphatic groups contain 1-12 aliphatic carbon atoms. As used herein, it is understood that an aliphatic group can also be bivalent (e.g., encompass a bivalent hydrocarbon chain that is saturated or contains one or more units of unsaturation, such as, for example, —CH₂—, —CH₂—CH₂—, —CH₂—CH₂—CH₂—, and so on). In some embodiments, aliphatic groups contain 1-6 aliphatic carbon atoms (e.g., C₁₋₆). In some embodiments, aliphatic groups contain 1-5 aliphatic carbon atoms (e.g., C₁₋₅). In other embodiments, aliphatic groups contain 1-4 aliphatic carbon atoms (e.g., C₁₋₄). In still other embodiments, aliphatic groups contain 1-3 aliphatic carbon atoms (e.g., C₁₋₃), and in yet other embodiments, aliphatic groups contain 1-2 aliphatic carbon atoms (e.g., C₁₋₂). In some embodiments, “cycloaliphatic” refers to a monocyclic C₃₋₈ hydrocarbon or a bicyclic C₇₋₁₀ hydrocarbon that is completely saturated or that contains one or more units of unsaturation, but which is not aromatic, that has a single point or more than one points of attachment to the rest of the molecule. Suitable aliphatic groups include, but are not limited to, linear or branched, substituted or unsubstituted alkyl, alkenyl, or alkynyl groups and hybrids thereof. A preferred aliphatic group is C₁₋₆ alkyl.

Alkyl: The term “alkyl”, used alone or as part of a larger moiety, refers to a saturated, optionally substituted straight or branched chain hydrocarbon group having (unless otherwise specified) 1-12, 1-10, 1-8, 1-6, 1-4, 1-3, or 1-2 carbon atoms (e.g., C₁₋₁₂, C₁₋₁₀, C₁₋₈, C₁₋₆, C₁₋₄, C₁₋₃, or C₁₋₂). Exemplary alkyl groups include methyl, ethyl, propyl, butyl, pentyl, hexyl, and heptyl.

Alkenyl: The term “alkenyl”, used alone or as part of a larger moiety, refers to an optionally substituted straight or branched chain or cyclic hydrocarbon group having at least one double bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkenyl groups include ethenyl, propenyl, butenyl, pentenyl, hexenyl, and heptenyl. The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentenyl, cyclohexenyl, and cycloheptenyl.

Alkynyl: The term “alkynyl”, used alone or as part of a larger moiety, refers to an optionally substituted straight or branched chain hydrocarbon group having at least one triple bond and having (unless otherwise specified) 2-12, 2-10, 2-8, 2-6, 2-4, or 2-3 carbon atoms (e.g., C₂₋₁₂, C₂₋₁₀, C₂₋₈, C₂₋₆, C₂₋₄, or C₂₋₃). Exemplary alkynyl groups include ethynyl, propynyl, butynyl, pentynyl, hexynyl, and heptynyl.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Aryl: The term “aryl” refers to monocyclic and bicyclic ring systems having a total of five to fourteen ring members (e.g., C₅-C₁₄), wherein at least one ring in the system is aromatic and wherein each ring in the system contains three to seven ring members. In some embodiments, an “aryl” group contains between six and twelve total ring members (e.g., C₆-C₁₂). The term “aryl” may be used interchangeably with the term “aryl ring”. In certain embodiments of the present invention, “aryl” refers to an aromatic ring system which includes, but not limited to, phenyl, biphenyl, naphthyl, anthracyl and the like, which may bear one or more substituents. Unless otherwise specified, “aryl” groups are hydrocarbons. In some embodiments, an “aryl” ring system is an aromatic ring (e.g., phenyl) that is fused to a non-aromatic ring (e.g., cycloalkyl). Examples of aryl rings include that are fused include

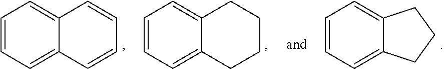

Associated: Two events or entities are “associated” with one another, as that term is used herein, if the presence, level and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids (e.g., sperm, sweat, tears), secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example, nucleic acids or proteins extracted from a sample or obtained by subjecting a primary sample to techniques such as amplification or reverse transcription of mRNA, isolation and/or purification of certain components, etc.

Carrier: As used herein, the term “carrier” refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Combination therapy: As used herein, the term “combination therapy” refers to those situations in which a subject is simultaneously exposed to two or more therapeutic regimens (e.g., two or more therapeutic agents or modality(ies)). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: As used herein, the term “cycloaliphatic” refers to a monocyclic C₃₋₈ hydrocarbon or a bicyclic C₇₋₁₀ hydrocarbon that is completely saturated or that contains one or more units of unsaturation, but which is not aromatic, that has a single point or more than one points of attachment to the rest of the molecule.

Cycloalkyl: As used herein, the term “cycloalkyl” refers to an optionally substituted saturated ring monocyclic or polycyclic system of about 3 to about 10 ring carbon atoms. Exemplary monocyclic cycloalkyl rings include cyclopropyl, cyclobutyl, cyclopentyl, cyclohexyl, and cycloheptyl.

Deoxyribonucleic Acid (DNA): As used herein, the term “DNA” refers to a polymeric molecule of nucleotides that are typically double-stranded and comprise adenine, cytosine, guanine and thymine, and a deoxyribose sugar backbone structure as specified in the definition “Nucleic Acid/Polynucleotide.” In some embodiments, DNA is linear DNA, plasmid DNA, minicircle DNA, nanoplasmid DNA, doggybone DNA, or a transposon.

Deoxyribonucleotide: As used herein, the term “deoxyribonucleotide” refers to unmodified and modified deoxyribonucleotides. For example, unmodified deoxyribonucleotides include the purine bases adenine (A) and guanine (G), and the pyrimidine bases cytosine (C) and thymine (T). Modified deoxyribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen or therapeutic regimen: Those skilled in the art will appreciate that the terms “dosing regimen” and “therapeutic regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Excipient: As used herein, the term “excipient” refers to a non-therapeutic agent that may be included in a pharmaceutical composition, for example, to provide or contribute to a desired consistency or stabilizing effect. Suitable pharmaceutical excipients include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Heteroaliphatic: The term “heteroaliphatic” or “heteroaliphatic group”, as used herein, denotes an optionally substituted hydrocarbon moiety having, in addition to carbon atoms, from one to five heteroatoms, that may be straight-chain (i.e., unbranched), branched, or cyclic (“heterocyclic”) and may be completely saturated or may contain one or more units of unsaturation, but which is not aromatic. The term “heteroatom” refers to nitrogen, oxygen, or sulfur, and includes any oxidized form of nitrogen or sulfur, and any quaternized form of a basic nitrogen. The term “nitrogen” also includes a substituted nitrogen. Unless otherwise specified, heteroaliphatic groups contain 1-10 carbon atoms wherein 1-3 carbon atoms are optionally and independently replaced with heteroatoms selected from oxygen, nitrogen, and sulfur. In some embodiments, heteroaliphatic groups contain 1-4 carbon atoms, wherein 1-2 carbon atoms are optionally and independently replaced with heteroatoms selected from oxygen, nitrogen, and sulfur. In yet other embodiments, heteroaliphatic groups contain 1-3 carbon atoms, wherein 1 carbon atom is optionally and independently replaced with a heteroatom selected from oxygen, nitrogen, and sulfur. Suitable heteroaliphatic groups include, but are not limited to, linear or branched, heteroalkyl, heteroalkenyl, and heteroalkynyl groups. For example, a 1- to 10 atom heteroaliphatic group includes the following exemplary groups: —O—CH₃, —CH₂—O—CH₃, —O—CH₂—CH₂—O—CH₂—CH₂—O—CH₃, and the like.

Heteroaryl: The terms “heteroaryl” and “heteroar-”, used alone or as part of a larger moiety, e.g., “heteroaralkyl”, or “heteroaralkoxy”, refer to monocyclic or bicyclic ring groups having 5 to 12 ring atoms (e.g., 5- to 6-membered monocyclic heteroaryl or 9- to 12-membered bicyclic heteroaryl); having 6, 10, or 14 TT-electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, imidazo[1,2-a]pyrimidinyl, imidazo[1,2-a]pyridyl, imidazo[4,5-b]pyridyl, imidazo[4,5-c]pyridyl, pyrrolopyridyl, pyrrolopyrazinyl, thienopyrimidinyl, triazolopyridyl, and benzoisoxazolyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring (i.e., a bicyclic heteroaryl ring having 1 to 3 heteroatoms). Nonlimiting examples include indolyl, isoindolyl, benzothienyl, benzofuranyl, dibenzofuranyl, indazolyl, benzimidazolyl, benzotriazolyl, benzothiazolyl, benzothiadiazolyl, benzoxazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, pyrido[2,3-b]-1,4-oxazin-3(4H)-one, 4H-thieno[3,2-b]pyrrole, and benzoisoxazolyl. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

Heteroatom: The term “heteroatom” as used herein refers to nitrogen, oxygen, or sulfur, and includes any oxidized form of nitrogen or sulfur, and any quaternized form of a basic nitrogen.

Heterocycle: As used herein, the terms “heterocycle”, “heterocyclyl”, “heterocyclic radical”, and “heterocyclic ring” are used interchangeably and refer to a stable 3- to 8-membered monocyclic, a 7- to 12-membered bicyclic, or a 10- to 16-membered polycyclic heterocyclic moiety that is either saturated or partially unsaturated, and having, in addition to carbon atoms, one or more, such as one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, azetidinyl, oxetanyl, tetrahydrofuranyl, tetrahydrothienyl, pyrrolidinyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted. A bicyclic heterocyclic ring also includes groups in which the heterocyclic ring is fused to one or more aryl rings. Exemplary bicyclic heterocyclic groups include indolinyl, isoindolinyl, benzodioxolyl, 1,3-dihydroisobenzofuranyl, 2,3-dihydrobenzofuranyl, tetrahydroquinolinyl,

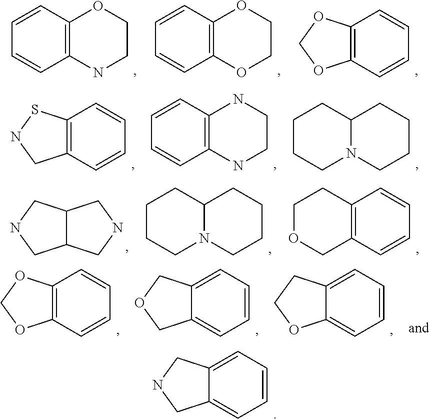

A bicyclic heterocyclic ring can also be a spirocyclic ring system (e.g., 7- to 11-membered spirocyclic fused heterocyclic ring having, in addition to carbon atoms, one or more heteroatoms as defined above (e.g., one, two, three or four heteroatoms)). A bicyclic heterocyclic ring can also be a bridged ring system (e.g., 7- to 11-membered bridged heterocyclic ring having one, two, or three bridging atoms.

Nanoparticle: As used herein, the term “nanoparticle” refers to a discrete entity of small size, e.g., typically having a longest dimension that is shorter than about 1000 nanometers (nm) and often is shorter than 500 nm, or even 100 nm or less. In many embodiments, a nanoparticle may be characterized by a longest dimension between about 1 nm and about 100 nm, or between about 1 μm and about 500 nm, or between about 1 nm and 1000 nm. In many embodiments, a population of microparticles is characterized by an average size (e.g., longest dimension) that is below about 1000 nm, about 500 nm, about 100 nm, about 50 nm, about 40 nm, about 30 nm, about 20 nm, or about 10 nm and often above about 1 nm. In many embodiments, a microparticle may be substantially spherical (e.g., so that its longest dimension may be its diameter). In some embodiments, a nanoparticle has a diameter of less than 100 nm as defined by the National Institutes of Health. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer.

Nucleic acid/Polynucleotide: As used herein, the term “nucleic acid” refers to a polymer of at least 10 nucleotides or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Nucleic acid particle: A “nucleic acid particle” can be used to deliver nucleic acid to a target site of interest (e.g., cell, tissue, organ, and the like). A nucleic acid particle may be formed from at least one cationic or cationically ionizable lipid or lipid-like material, at least one cationic polymer such as protamine, or a mixture thereof and nucleic acid. Nucleic acid particles include lipid nanoparticle (LNP)-based and lipoplex (LPX)-based formulations.

Nucleotide: As used herein, the term “nucleotide” refers to its art-recognized meaning. When a number of nucleotides is used as an indication of size, e.g., of a polynucleotide, a certain number of nucleotides refers to the number of nucleotides on a single strand, e.g., of a polynucleotide.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intra-arterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

Partially unsaturated: As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amount appropriate for administration in a therapeutic or dosing regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66:1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Physiological conditions: as used herein, has its art-understood meaning referencing conditions under which cells or organisms live and/or reproduce. In some embodiments, the term refers to conditions of the external or internal milieu that may occur in nature for an organism or cell system. In some embodiments, physiological conditions are those conditions present within the body of a human or non-human animal, especially those conditions present at and/or within a surgical site. Physiological conditions typically include, e.g., a temperature range of 20-40° C., atmospheric pressure of 1, pH of 6-8, glucose concentration of 1-20 mM, oxygen concentration at atmospheric levels, and gravity as it is encountered on earth. In some embodiments, conditions in a laboratory are manipulated and/or maintained at physiologic conditions. In some embodiments, physiological conditions are encountered in an organism.

Polycyclic: As used herein, the term “polycyclic” refers to a saturated or unsaturated ring system having two or more rings (for example, heterocyclyl rings, heteroaryl rings, cycloalkyl rings, or aryl rings), having between 7 and 20 atoms, in which one or more carbon atoms are common to two adjacent rings. For example, in some embodiments, a polycyclic ring system refers to a saturated or unsaturated ring system having three or more rings (for example, heterocyclyl rings, heteroaryl rings, cycloalkyl rings, or aryl rings), having between 14 and 20 atoms, in which one or more carbon atoms are common to two adjacent rings. The rings in a polycyclic ring system may be fused (i.e., bicyclic or tricyclic), spirocyclic, or a combination thereof.

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics).

Polymer. As used herein, the term “polymer” refers to a composition comprising one or more molecules that comprise repeating units of one or more monomers. As used herein, “polymer” and “polymer composition” are used interchangeably, and unless otherwise specified, refer to a composition of polymer molecules. A person of skill in the art will appreciate that a polymer composition comprises polymer molecules having molecules of different lengths (e.g., comprising varying amounts of monomers). Polymer compositions described herein are characterized by one or more of a number average molecular weight (Mₙ), a weight average molecular weight (M_{w}), and/or a polydispersity index (PDI). Polymers described herein can also be characterized by the degree of polymerization (DP), which refers to the number of monomer units in the polymer. A polymer described herein can be a homopolymer, a heteropolymer, or a block-co-polymer. As used herein, a “homopolymer” refers to a polymer having a single type of monomer repeating throughout a polymer chain, e.g., -A-A-A-A-. As used herein, a “heteropolymer” refers to a polymer having more than one type (e.g., two or more) types of monomers present throughout a polymer chain, e.g., -A-B-A-B-A-. As used herein, a “block-co-polymer” refers to a polymer having an arrangement of blocks of polymerized monomers, e.g., -A-A-A-A-B-B-B-B- (a di block polymer) or -A-A-A-A-B-B-B-B-A-A-A- (a tri block polymer). Polymers described herein can be linear or branched. As used herein, a “linear polymer” refers to a polymer in which the molecules form long chains without branches or cross-linked structures. As used herein, a “branched polymer” refers to a polymer comprising a polymer backbone with one or more additional monomers, or chains of monomers, extending from polymer backbone that can form, for example, branches or cross-linked structures.

Prevent or prevention: As used herein, the terms “prevent” or “prevention”, when used in connection with the occurrence of a disease, disorder, and/or condition, refer to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments where an RNA is a mRNA. In some embodiments where an RNA is a mRNA, a RNA typically comprises at its 3′ end a poly(A) region. In some embodiments where an RNA is a mRNA, an RNA typically comprises at its 5′ end an art-recognized cap structure, e.g., for recognizing and attachment of a mRNA to a ribosome to initiate translation. In some embodiments, a RNA is a synthetic RNA. Synthetic RNAs include RNAs that are synthesized in vitro (e.g., by enzymatic synthesis methods and/or by chemical synthesis methods). As used herein, “monomeric RNA” refers to an individual RNA molecule that is not an aggregate, a dimer, trimer, or oligomer of RNA.

Sample: As used herein, the term “sample” typically refers to an aliquot of material obtained or derived from a source of interest. In some embodiments, a source of interest is a biological or environmental source. In some embodiments, a source of interest may be or comprise a cell, tissue, or organism, such as a microbe, a plant, or an animal (e.g., a human). In some embodiments, a source of interest is or comprises biological tissue or fluid. In some embodiments, a source of interest may be or comprise a preparation generated in a production run. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample.

Substituted or optionally substituted: As described herein, compounds of the invention may contain “optionally substituted” moieties. In general, the term “substituted,” whether preceded by the term “optionally” or not, means that one or more hydrogens of the designated moiety are replaced with a suitable substituent. “Substituted” applies to one or more hydrogens that are either explicit or implicit from the structure

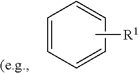

refers to at least

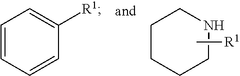

refers to at least

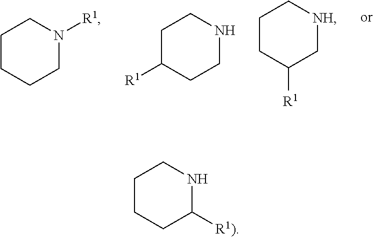

Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes provided herein. Groups described as being “substituted” preferably have between 1 and 4 substituents, more preferably 1 or 2 substituents. Groups described as being “optionally substituted” may be unsubstituted or be “substituted” as described above.

Suitable monovalent substituents on a substitutable carbon atom of an “optionally substituted” group are independently halogen; —(CH₂)₀₋₄R^{◯}; —(CH₂)₀₋₄OR^{◯}; —O(CH₂)₀₋₄R^{◯}, —O—(CH₂)₀₋₄C(O)OR^{◯}; —(CH₂)₀₋₄CH(OR^{◯})₂; —(CH₂)₀₋₄SR^{◯}; —(CH₂)₀₋₄Ph, which may be substituted with R^{◯}; —(CH₂)₀₋₄O(CH₂)₀₋₁Ph which may be substituted with R^{◯}; —CH═CHPh, which may be substituted with R^{◯}; —(CH₂)₀₋₄O(CH₂)₀₋₁-pyridyl which may be substituted with R^{◯}; —NO₂; —CN; —N₃; (CH₂)₀₋₄N(R^{◯}₂); —(CH₂)₀₋₄N(R^{◯})C(O)R^{◯}); —N(R^{◯})C(S)R^{◯}); —(CH₂)₀₋₄N(R^{◯})C(O)NR^{◯}₂); N(R^{◯})C(S)NR^{◯}₂); —(CH₂)₀₋₄N(R^{◯})C(O)OR^{◯}; —N(R^{◯})N(R^{◯})C(O)R^{◯}; N(R^{◯})N(R^{◯})C(O)NR^{◯}₂; N(R^{◯})N(R^{◯})C(O)OR^{◯}; —(CH₂)₀₋₄C(O)R^{◯}; C(S)R^{◯}; —(CH₂)₀₋₄C(O)OR^{◯}; —(CH₂)₀₋₄C(O)SR^{◯}; (CH₂)₀₋₄C(O)OSiR^{◯}₃; —(CH₂)₀₋₄OC(O)R^{◯}; —OC(O)(CH₂)₀₋₄SR^{◯}; —(CH₂)₀₋₄SC(O)R^{◯}; —(CH₂)₀₋₄C(O)NR^{◯}₂; —C(S)NR^{◯}₂; —C(S)SR^{◯}; —SC(S)SR^{◯}, (CH₂)₀₋₄OC(O)NR^{◯}₂; C(O)N(OR^{◯})R^{◯}; —C(O)C(O)R^{◯}; —C(O)CH₂C(O)R^{◯}; —C(NOR^{◯})R^{◯}; (CH₂)₀₋₄SSR^{◯}; —(CH₂)₀₋₄S(O)₂R^{◯}; —(CH₂)₀₋₄S(O)₂OR^{◯}; —(CH₂)₀₋₄OS(O)₂R^{◯}; —S(O)₂NR^{◯}₂; (CH₂)₀₋₄S(O)R^{◯}; N(R^{◯})S(O)₂NR^{◯}₂; —N(R^{◯})S(O)₂R^{◯}; —N(OR^{◯})R^{◯}; —C(NH)NR^{◯}₂; —P(O)₂R^{◯}; P(O)R^{◯}₂; OP(O)R^{◯}₂; —OP(O)(OR^{◯})₂; SiR^{◯}₃; —(C₁₋₄ straight or branched alkylene)O—N(R^{◯})₂; or —(C₁₋₄ straight or branched alkylene) C(O)O—N(R^{◯})₂, wherein each R^{◯} may be substituted as defined below and is independently hydrogen, C₁₋₆ aliphatic, —CH₂Ph, —O(CH₂)₀₋₁Ph, —CH₂—(5- to 6-membered heteroaryl ring), or a 3- to 6-membered saturated, partially unsaturated, or aryl ring having 0-4 heteroatoms independently selected from nitrogen, oxygen, or sulfur, or, notwithstanding the definition above, two independent occurrences of R^{◯}, taken together with their intervening atom(s), form a 3- to 12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{◯} (or the ring formed by taking two independent occurrences of R^{◯} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂, O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur. Suitable divalent substituents on a saturated carbon atom of R^{◯} include ═O and ═S.

Suitable divalent substituents on a saturated carbon atom of an “optionally substituted” group include the following: ═O (“oxo”), ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include:—O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R′ include halogen, —R^{●}, (haloR^{●}, OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, S(O)₂R^{†}, S(O)₂NR^{†}₂, —C(S)NR^{†}₂, —C(NH)NR^{†}₂, or —N(R^{†}) S(O)₂R^{†}; wherein each R^{†} is independently hydrogen, C₁₋₆ aliphatic which may be substituted as defined below, unsubstituted-OPh, or an unsubstituted 3- to 6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, (haloR^{●}), —OH, —OR^{●}, —O(haloR^{●}, —CN, —C(O)OH, —C(O)OR^{●}, —NH₂, —NHR^{●}, —NR^{●}₂, or NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3- to 6-membered saturated, partially unsaturated, or aryl ring having 0-4 heteroatoms independently selected from nitrogen, oxygen, or sulfur.

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer.

Those of ordinary skill in the art, reading the present disclosure, will appreciate that certain small molecule compounds described herein, for example, may be provided and/or utilized in any of a variety of forms such as, for example, crystal forms (e.g., polymorphs, solvates, etc), salt forms, protected forms, pro-drug forms, ester forms, isomeric forms (e.g., optical and/or structural isomers), isotopic forms, etc.

Those of ordinary skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers; in some embodiments, such a small molecule may be utilized in accordance with the present disclosure in a racemic mixture form.

Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms.

Those of skill in the art will appreciate that certain small molecule compounds have structures that permit isotopic substitution (e.g., ²H or ³H for H; ¹¹C, ¹³C or ¹⁴C for ¹²C; ¹³N or ¹⁵N for ¹⁴N; ¹⁷O or ¹⁸O for ¹⁶O; ³⁶Cl for ³⁵Cl or ³⁷Cl; ¹⁸F for ¹⁹F; ¹³¹I for ¹²⁷I; etc.). In some embodiments, such a small molecule may be utilized in accordance with the present disclosure in one or more isotopically modified forms, or mixtures thereof.

In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form.

In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Those skilled in the art will further appreciate that, in small molecule structures, the symbol [symbol] , as used herein, refers to a point of attachment between two atoms.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### Complexes

The present disclosure provides, among other things, complexes for delivery of a nucleic acid, e.g., RNA. In some embodiments, the present disclosure provides a complex comprising a cationic polymer, an anionic polymer, and RNA. In some embodiments, the present disclosure provides a complex comprising a cationic polymer, an anionic polymer, and RNA, wherein the cationic polymer and the RNA form a core complex encapsulated by the anionic polymer. In some embodiments, a complex described herein comprises a monomeric RNA molecule.

As described herein, reference to a “binary formulation” or “binary particle” is also intended to refer to a “core complex” as described throughout the present application. That is, a binary formulation comprises a cationic polymer and a nucleic acid. A “ternary formulation” or “ternary particle” is also intended to refer to a complex comprising a cationic polymer, a nucleic acid, and an anionic polymer, wherein the cationic polymer and the nucleic acid form a core complex that is encapsulated by an anionic polymer.

The presently described complexes exhibit numerous benefits over previous formulations due, at least in part, to the presence of an anionic polymer that acts as a shell to encapsulate a core complex of a cationic polymer and RNA. Without being bound by theory, it is understood that the anionic polymer induces release of RNA from the core complex by displacement of negatively charged phosphates.

In some embodiments, a complex comprises a linear cationic polymer, an anionic polymer, and RNA. In some embodiments, a complex comprises a linear cationic polymer, an anionic polymer, and a monomeric RNA molecule. In some embodiments, a complex comprises a linear cationic polymer, an anionic polymer, and RNA, wherein the linear cationic polymer and the RNA form a core complex encapsulated by the anionic polymer. In some embodiments, a complex comprises a linear cationic polymer, an anionic polymer, and a monomeric RNA molecule, wherein the linear cationic polymer and the monomeric RNA molecule form a core complex encapsulated by the anionic polymer. In some embodiments, a complex comprises a linear cationic polymer, an anionic polymer, and RNA, wherein the linear cationic polymer and the RNA form a core complex encapsulated by the anionic polymer, and wherein the cationic polymer is a linear poly(ethyleneimine). In some embodiments, a complex comprises a linear cationic polymer, an anionic polymer, and a monomeric RNA molecule, wherein the linear cationic polymer and the monomeric RNA molecule form a core complex encapsulated by the anionic polymer, and wherein the cationic polymer is a linear poly(ethyleneimine). In some embodiments, a complex comprises a linear cationic polymer, an anionic polymer, and RNA, wherein the linear cationic polymer and the RNA form a core complex encapsulated by the anionic polymer, wherein the cationic polymer is a linear poly(ethyleneimine), and the anionic polymer is a block co-polymer comprising blocks of polymers selected from poly-L-glutamic acid, poly-L-aspartic acid, and poly-2-(2-(2-aminoethoxy) ethoxy) acetic acid. In some embodiments, a complex comprises a linear cationic polymer, an anionic polymer, and a monomeric RNA molecule, wherein the linear cationic polymer and the monomeric RNA molecule form a core complex encapsulated by the anionic polymer, wherein the cationic polymer is a linear poly(ethyleneimine), and the anionic polymer is a block co-polymer comprising blocks of polymers selected from poly-L-glutamic acid, poly-L-aspartic acid, and poly-2-(2-(2-aminoethoxy) ethoxy) acetic acid.

### Core Complexes

As described generally herein, in some embodiments, a complex comprises a core complex (also referred to as a binary particle) comprising a cationic polymer and RNA (e.g., monomeric RNA, as described herein). In some embodiments, a complex comprises a core complex comprising a cationic polymer, RNA, and an additional nucleic acid (e.g., DNA). Examples of cationic polymers and conditions suitable for forming core complexes are described in PCT App. Pub. No. WO 2021/001417, which is incorporated herein by reference in its entirety. Such complexes are characterized by an “N/P ratio,” which is the molar ratio of cationic (nitrogen) groups (the “N” in N/P) in the cationic polymer to the anionic (phosphate) groups (the “P” in N/P) in RNA. It is understood that a cationic group is one that is either in cationic form (e.g., N⁺), or one that is ionizable to become cationic. Use of a single number in an N/P ratio (e.g., an N/P ratio of about 5) is intended to refer to that number over 1, e.g., an N/P ratio of about 5 is intended to mean a ratio of about 5:1.

In some embodiments, an N/P ratio for a core complex described herein is greater than or equal to about 5. In some embodiments, an N/P ratio for a core complex described herein is greater than or equal to about 10. In some embodiments, an N/P ratio for a core complex described herein is greater than or equal to about 12. In some embodiments, an N/P ratio for a core complex described herein is greater than or equal to about 20. In some embodiments, an N/P ratio for a core complex described herein is greater than or equal to about 40. In some embodiments, an N/P ratio for a core complex described herein is greater than or equal to about 70. In some embodiments, an N/P ratio for a core complex described herein is greater than or equal to about 100. In some embodiments, an N/P ratio for a core complex described herein is greater than or equal to about 200. In some embodiments, an N/P ratio for a core complex described herein is greater than or equal to about 300. In some embodiments, an N/P ratio for a core complex described herein is from about 5 to about 20 (e.g., about 5, about 6, about 7, about 8, about 9, about 10, about 11, about 12, about 13, about 14, about 15, about 16, about 17, about 18, about 19, or about 20). In some embodiments, an N/P ratio for a core complex described herein is from about 10 to about 50. In some embodiments, an N/P ratio for a core complex described herein is from about 10 to about 70. In some embodiments, an N/P ratio for a core complex described herein is from about 10 to about 120. In some embodiments, an N/P ratio for a core complex described herein is from about 40 to about 70. In some embodiments, an N/P ratio for a core complex described herein is from about 40 to about 120 (e.g., about 40, about 50, about 60, about 70, about 80, about 90, about 100, about 110, or about 120). In some embodiments, an N/P ratio for a core complex described herein is from about 70 to about 120. In some embodiments, an N/P ration is greater than or equal to 48. In some embodiments, an N/P ratio is from about 48 to about 300. In some embodiments, an N/P ratio is from about 60 to about 200. In some embodiments, an N/P ratio is from about 80 to about 150.

In some embodiments, a core complex described herein is characterized by an RNA concentration. In some embodiments, an RNA concentration in a core complex is greater than or equal to about 0.05 mg/mL. In some embodiments, an RNA concentration in a core complex is greater than or equal to about 0.1 mg/mL. In some embodiments, an RNA concentration in a core complex is greater than or equal to about 0.25 mg/mL. In some embodiments, an RNA concentration in a core complex is greater than or equal to about 0.5 mg/mL. In some embodiments, an RNA concentration in a core complex is from about 0.01 mg/mL to about 0.5 mg/mL. In some embodiments, an RNA concentration in a core complex is from about 0.1 mg/ml to about 0.25 mg/mL. In some embodiments, an RNA concentration in a core complex is about 0.1 mg/mL, about 0.15 mg/mL, about 0.2 mg/mL, about 0.25 mg/mL, about 0.3 mg/mL, about 0.35 mg/mL, about 0.4 mg/mL, about 0.45 mg/mL, or about 0.5 mg/mL. In some embodiments, an RNA concentration in a core complex is about 0.1 mg/mL.

In some embodiments, an RNA concentration in a core complex is about 0.1 mg/mL, and an N/P ratio is greater than or equal to about 45. In some embodiments, an RNA concentration in a core complex is about 0.1 mg/mL, and an N/P ratio is from about 48 to about 300. In some embodiments, an RNA concentration in a core complex is about 0.1 mg/mL, and an N/P ratio is from about 60 to about 200. In some embodiments, an RNA concentration in a core complex is about 0.1 mg/mL, and an N/P ratio is from about 80 to about 150.

In some embodiments, an RNA concentration in a core complex is about 0.05 mg/mL, and an N/P ratio is greater than or equal to about 5. In some embodiments, an RNA concentration in a core complex is about 0.05 mg/mL, and an N/P ratio is greater than or equal to about 12. In some embodiments, an RNA concentration in a core complex is about 0.05 mg/mL, and an N/P ratio is greater than or equal to about 45. In some embodiments, an RNA concentration in a core complex is about 0.05 mg/mL, and an N/P ratio is greater than about 60.

In some embodiments, a core complex described herein is prepared according to a process comprising a step of contacting a solution comprising RNA with a solution comprising a cationic polymer at a ratio of about 3:1 (v:v) or greater. In some embodiments, a core complex described herein is prepared according to a process comprising a step of contacting a solution comprising RNA with a solution comprising a cationic polymer at a ratio of about 20:1 (v:v) or greater. In some embodiments, a core complex described herein is prepared according to a process comprising a step of contacting a solution comprising RNA with a solution comprising a cationic polymer at a ratio of about 50:1 (v:v) or greater. In some embodiments, a core complex described herein is prepared according to a process comprising a step of contacting a solution comprising RNA with a solution comprising a cationic polymer at a ratio of about 99:1 (v:v) or greater.

In some embodiments, a core complex described herein comprises monomeric RNA. “Monomeric RNA,” as used herein, refers to RNA that is an individual RNA molecule, and not an aggregate, or as a dimer, trimer, or oligomer of RNA. Therefore, reference to a complex comprising monomeric RNA is understood to be a complex that with a single RNA molecule that is not aggregate, or as a dimer, trimer, or oligomer of RNA.

In some embodiments, a core complex further comprises DNA.

It is understood that complexes described herein are included as part of a solution or formulation. As such, many complexes will make up a solution or formulation. Characterization of any complex within is intended to represent either an average of all complexes within a given solution or formulation, as well as individual complexes when characterized according to methods known in the art.

### Cationic Polymers

As described generally above, a complex described herein comprises a cationic polymer. In some embodiments, a cationic polymer is a polycationic polymer, e.g., a polymer having one or more cationic or ionizable groups. As described herein, a “cationic” group is a group having a net positive charge. As used herein, an “ionizable” group is a group that may have a neutral charge at a certain pH, but may become charged (e.g., cationic) at a different pH. For example, in some embodiments, an ionizable group becomes cationic (i.e., positively charged) at physiological pH (e.g., a pH of about 7.4). Accordingly, it is understood that reference to a cationic polymer refers to both the neutral form and the charged (i.e., ionic) form. A person of skill in the art will also appreciate that cationic groups described herein can also exist as a salt (e.g., a pharmaceutically acceptable salt) that comprises a cationic group and one or more suitable counterions. For example, in some embodiments, a cationic group described herein comprises ammonium chloride. Suitable counterions include halogens (e.g., Br⁻, Cl⁻, I⁻, F⁻), acetates (e.g., C(O)O⁻), and the like. For additional examples, see the definition for pharmaceutically acceptable salts described herein.

In some embodiments, one or more cationic or ionizable groups comprise a nitrogen atom. Cationic polymers useful for preparing complexes described herein can be homopolymers heteropolymers, or block-co-polymers.

In some embodiments, a cationic polymer is a homopolymer. In some embodiments, a cationic polymer is a homopolymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

It is understood that cationic polymers described herein can be linear or branched. In some embodiments, a cationic polymer is linear. In some embodiments, a cationic polymer is a linear polymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

In some embodiments, a cationic polymer is a branched polymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a cationic polymer is linear poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

In some embodiments, a cationic polymer is poly(ethylenimine). In some embodiments, a cationic polymer is poly(propylenimine). In some embodiments, a cationic polymer is polybrene. In some embodiments, a cationic polymer is polyallylamine. In some embodiments, a cationic polymer is polyvinylamine. In some embodiments, a cationic polymer is polyamidoamine. In some embodiments, a cationic polymer is poly-L-lysine. In some embodiments, a cationic polymer is poly-L-arginine. In some embodiments, a cationic polymer is poly-L-histidine. In some embodiments, a cationic polymer is poly(2-aminoethyl methacrylate).

In some embodiments, a cationic polymer is linear poly(ethylenimine). In some embodiments, a cationic polymer is linear poly(propylenimine). In some embodiments, a cationic polymer is linear polybrene. In some embodiments, a cationic polymer is linear polyallylamine. In some embodiments, a cationic polymer is linear polyvinylamine. In some embodiments, a cationic polymer is linear polyamidoamine. In some embodiments, a cationic polymer is linear poly-L-lysine. In some embodiments, a cationic polymer is linear poly-L-arginine. In some embodiments, a cationic polymer is linear poly-L-histidine. In some embodiments, a cationic polymer is linear poly(2-aminoethyl methacrylate).

In some embodiments, a cationic polymer is a heteropolymer comprising copolymers of one or more of poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a cationic polymer is a heteropolymer comprising poly(ethylenimine) and poly(propylenimine). In some embodiments, a cationic polymer is a linear heteropolymer comprising copolymers of one or more of poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a cationic polymer is a heteropolymer comprising poly(ethylenimine) and poly(propylenimine).

In some embodiments, a cationic polymer is a block-co-polymer comprising blocks of polymers selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof. In some embodiments, a cationic polymer is a block-co-polymer of poly(ethylenimine) and poly(propylenimine) (i.e., poly(ethylenimine)-block-poly(propylenimine)). In some embodiments, a cationic polymer is a linear block-co-polymer comprising blocks of polymers selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate). In some embodiments, a cationic polymer is a linear block-co-polymer of poly(ethylenimine) and poly(propylenimine) (i.e., poly(ethylenimine)-block-poly(propylenimine)).

In some embodiments, a cationic polymer has between 2 and 2000 repeating monomeric units. In some embodiments, a cationic polymer has between 100 and 2000 repeating monomeric units. In some embodiments, a cationic polymer has between 500 and 2000 repeating monomeric units. In some embodiments, a cationic polymer has between 1000 and 2000 repeating monomeric units. In some embodiments, a cationic polymer has between 1500 and 2000 repeating monomeric units. In some embodiments, a cationic polymer comprises about 500, about 600, about 700, about 800, about 900, about 1000, about 1100, about 1200, about 1300, about 1400, about 1500, about 1600, about 1700, about 1800, about 1900, or about 2000 monomeric units.

In some embodiments, a cationic polymer is a polymer or copolymer comprising blocks of formula I and/or II:

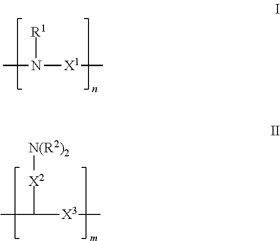

or a pharmaceutically acceptable salt thereof, wherein:
R¹ is H, —C(O)-optionally substituted C₁-C₆ aliphatic, optionally substituted C₁-C₆ aliphatic or G;
each R² is independently H, optionally substituted C₁-C₆ aliphatic, or G;
X¹ and X³ are each independently C₁-C₆ aliphatic;
X² is a bond or optionally substituted C₁-C₆ aliphatic;
m is an integer between 2 and 2000;
n is an integer between 2 and 2000;
each G is independently:

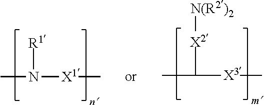

R^{1′} is H, —C(O)-optionally substituted C₁-C₆ aliphatic, optionally substituted C₁-C₆ aliphatic, or G′;
each R^{2′} is independently H, optionally substituted C₁-C₆ aliphatic, or G′;
X^{1′} and X^{3′} are each independently optionally substituted C₁-C₆ aliphatic;
X^{2′} is a bond or optionally substituted C₁-C₆ aliphatic;
n′ is an integer between 2 and 2000;
m′ is an integer between 2 and 2000;
each G′ is independently:

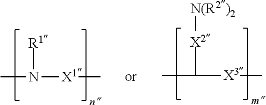

R^{1″} is H, —C(O)-optionally substituted C₁-C₆ aliphatic, or optionally substituted C₁-C₆ aliphatic;
each R^{2″} is independently H or C₁-C₆ aliphatic;
X^{1″} and X^{3″} are each independently optionally substituted C₁-C₆ aliphatic;
X^{2″} is a bond or optionally substituted C₁-C₆ aliphatic;
n″ is an integer between 2 and 2000; and
m″ is an integer between 2 and 2000.

In some embodiments, a cationic polymer is a polymer described herein, having a number average molecular weight (Mₙ) of about 600 Daltons (Da) to about 400,000 Da. In some embodiments, a cationic polymer has a Mₙ of about 1,000 Da to about 300,000 Da. In some embodiments, a cationic polymer has a Mₙ of about 10,000 to about 250,000 Da. In some embodiments, a cationic polymer has a Mₙ of about 10,000 to about 120,000 Da. In some embodiments, a cationic polymer has a Mₙ of about 20,000 to about 120,000 Da.

In some embodiments, provided compounds are provided and/or utilized in a salt form (e.g., a pharmaceutically acceptable salt form). Reference to a compound provided herein is understood to include reference to salts thereof, unless otherwise indicated.

### Polyamine Derivatives (Viromers)

In some embodiments, a complex comprises a cationic polymer, an anionic polymer, and RNA, wherein the cationic polymer is or comprises a polyamine derivative. As described herein, a polyamine derivative is also referred to as a “viromer.” In some embodiments, a complex comprises a cationic polymer, an anionic polymer, and a monomeric RNA molecule, wherein the cationic polymer is or comprises a polyamine derivative. In some embodiments, a complex comprises a cationic polymer, an anionic polymer, RNA, and DNA, wherein the cationic polymer is or comprises a polyamine derivative.

In some embodiments, a complex comprises a cationic polymer, an anionic polymer, and RNA, wherein the cationic polymer is or comprises a polyamine derivative, and the polyamine derivative and the RNA form a core complex that is encapsulated by the anionic polymer. In some embodiments, a complex comprises a cationic polymer, an anionic polymer, and a monomeric RNA molecule, wherein the cationic polymer is a polyamine derivative, and the polyamine derivative and the monomeric RNA molecule form a core complex that is encapsulated by the anionic polymer.

In some embodiments, a complex described herein comprises a polyamine derivative, e.g., a carboxylated polyamine derivative. Without being bound by theory, it is understood that polyamines form polycations in solution, which facilitates the complex formation with polyanions such as nucleic acids.

In some embodiments, a polyamine derivative comprises:
a polyamine moiety comprising a plurality of amino groups;
a plurality of carboxylated substituents comprising a carboxyl group bonded via a hydrophobic linker to amino groups of said polyamine moiety; and
a plurality of hydrophobic substituents bonded to amino groups of said polyamine moiety.

In some embodiments, a polyamine derivative comprises:
a polyamine moiety comprising a plurality of amino groups;
a plurality of carboxylated substituents comprising a carboxyl group bonded via a hydrophobic linker to amino groups of said polyamine moiety, wherein each of said carboxylated substituents comprises from 6 to 40 carbon atoms, preferably from 6 to 20 carbon atoms, and more preferably from 8 to 16 carbon atoms, and each of said hydrophobic linker may comprise from 1 to 3 heteroatoms selected from O, N, and S; and
a plurality of hydrophobic substituents bonded to amino groups of said polyamine moiety, wherein each of said hydrophobic substituents comprises at least 2 carbon atoms, preferably from 6 to 40 carbon atoms, and may comprise from 1 to 3 heteroatoms selected from O, N, and S provided said hydrophobic substituent has at least 6 carbon atoms.

In some embodiments, each of said carboxylated substituents of said polyamine derivative comprises any one or more of the following moieties as said hydrophobic linker: alkylene, alkenylene, alkynylene, cycloalkylene, cycloalkenylene, arylene, and combinations thereof; and/or each of said hydrophobic substituents of said polyamine derivative comprises any one or more of the following moieties: alkyl, alkenyl, alkynyl, cycloalkyl, cycloalkenyl, aryl, and combinations thereof.

In some embodiments, a polyamine derivative which is useful herein as delivery vehicle for polyanions is a polyalkylenimine derivative having one or more carboxyalkyl substituents comprising from 6 to 40 carbon atoms, and one or more hydrophobic substituents selected from hydrocarbon substituents having at least 2 carbon atoms, preferably from 6 to 40 carbon atoms, wherein each of said hydrophobic substituents may be or may comprise an alkyl group and/or each of said hydrophobic substituents may be or may comprise an aryl group.

In some embodiments, the polyalkylenimine is selected from the group consisting of polyethylenimines, polypropylenimines, and polybutylenimines.

In some embodiments, the polyamine moiety of said polyamine derivative may comprise from 4 to 20000 nitrogen atoms, more preferably from 6 to 10000 nitrogen atoms, e.g., from 6 to 1000 nitrogen atoms, or from 6 to 100 nitrogen atoms per polyamine molecule. In some embodiments, the polyamine moiety of said polyamine derivative may be a branched polyamine, preferably a branched polyalkylenimine.

In some embodiments, a carboxylated substituent comprises one or two carboxyl groups, preferably one carboxyl group. In some embodiments, each carboxylated substituent comprises from 6 to 40 carbon atoms, preferably from 6 to 20 carbon atoms, and more preferably from 8 to 16 carbon atoms. The hydrophobic linkers of said carboxylated substituents may comprise from 1 to 3, preferably, 1 or 2, heteroatoms selected from O, N, and S. Preferably, the heteroatoms are selected from O and S. In some embodiments, 1 or 2 heteroatoms selected from O, N and S, preferably O and S, are contained in the hydrophobic linker. In some embodiments, carboxylated substituents may be carboxyhydrocarbyl groups. In some embodiments, carboxylated substituents are carboxyheterohydrocarbyl groups comprising from 1 to 3 heteroatoms selected from O, N, and S, preferably selected from O and S.

In some embodiments, among the plurality of carboxylated substituents of a molecule of said polyamine derivative, there may be exclusively carboxyhydrocarbyl groups, exclusively carboxyheterohydrocarbyl groups, or there may be carboxyhydrocarbyl groups and carboxyheterohydrocarbyl groups. In some embodiments, the plurality of carboxylated substituents are all carboxyhydrocarbyl groups. In some embodiments, the plurality of carboxylated substituents are all carboxyheterohydrocarbyl groups.

In some embodiments, carboxylated substituents are carboxyhydrocarbyl groups, and the hydrocarbyl moieties of said carboxyhydrocarbyl groups may be saturated aliphatic moieties, cycloaliphatic moieties, aromatic moieties, or moieties comprising two or more moieties from the aforementioned list.

Examples of the carboxyhydrocarbyl groups are carboxyalkyl groups, carboxyalkenyl groups, carboxyalkynyl groups, carboxycycloalkyl groups, carboxycycloalkenyl groups, carboxyalkylcycloalkyl groups, carboxycycloalkylalkyl groups, carboxyalkylcycloalkylalkyl groups, carboxyaryl groups, carboxyalkylaryl groups, carboxyarylalkyl groups, and carboxyalkylarylalkyl groups. It is possible to replace 1, 2 or 3, preferably 1 or 2, of the carbon atoms of the hydrocarbyl moieties of the carboxylated substituents by oxygen, nitrogen or sulfur, thereby forming carboxyheterohydrocarbyl moieties. It is understood that any such formal replacement by a heteroatom will include adjustment of bound hydrogen atoms to adjust to the valency of the exchanged heteroatom. In preferred embodiments, such carboxyheterohydrocarbyl moieties comprise one or more functional group selected from —O—, —S—, —N(H) C(O)—, —C(O)O— —OC(O)N(H)—, —C(O)—, —C(O)—N(H)—, —N(H)—C(O)—O—, —O—C(O)—, or —S—S— in the hydrophobic linker.

In some embodiments, the hydrophobic linkers are or comprise alkylene groups such as linear or branched alkylene groups, or the linkers are or comprise cycloalkylene groups. Alkylene groups may be n-alkylene or isoalkylene groups. Examples of alkylene groups are propylene, butylene, pentylene, hexylene, heptylene, octylene, nonylene, decylene, undecylene, dodecylene, tetradecylene or hexadecylene groups. Examples of cycloalkylene groups are cyclopentylene, cyclohexylene and cycloheptylene groups. Examples of alkylcycloalkyl groups are methylcyclopentylene, ethylcyclopentylene, propylcyclopentylene, butylcyclopentylene, pentylcyclopentylene, hexylcylopentylene, methylcyclohexylene, ethylcyclohexylene, propylcyclohexylene, butylcyclohexylene, pentylcyclohexylene and hexylcylohexylene. One or more of these may be combined in a hydrophobic linker.

In some embodiments, the carboxylated substituents are or comprise carboxyalkyl or carboxycycloalkyl groups comprising from 6 to 20 carbon atoms. Such carboxylated substituents may be selected from the group consisting of carboxy-n-alkyl groups, branched carboxyalkyl groups or cyclic carboxyalkyl groups and their constitution or conformation isomers. In a preferred embodiment, the carboxylalkyl groups are radicals of acids selected from hexanoic acid, heptanoic acid, octanoic acid, nonanoic acid, decanoic acid, undecanoic acid, dodecanoic acid, tetradecanoic acid, hexadecanoic acid, 2-cyclohexylacetic acid, 4-cyclohexylbutyric acid, 6-cyclohexylhexanoic acid, 2-(2′, 3′ or 4′ ethylcyclohexyl)-acetic acid or 4-(2′, 3′ or 4′ ethylcyclohexyl)-butyric acid or 6-(2′, 3′ or 4′ ethylcyclohexyl)-hexanoic acid.

In some embodiments, the hydrophobic linkers are or comprise arylene groups and have from 6 to 20 carbon atoms. Aryl groups forming said arylene groups include aromatic hydrocarbyl groups (carbon-only aryl groups) and aromatic heterohydrocarbyl groups (heteroaryl groups). Examples of the former are phenyl, naphthyl, anthracenyl and phenanthryl. In some embodiments, nitrogen-containing heteroaryl groups have a pK value of <5 for avoiding additional cationic charges at neutral pH. Examples of such nitrogen-containing heteroaryl groups are indolyl groups pyrazinyl groups, pyridazinyl groups, pyrimidinyl groups, cinnolinyl groups, phthalazinyl groups and purinyl groups. In some embodiments, oxygen-containing heterohydrocarbyl groups that form hydroxy groups have a pK>12 for avoiding negative charges at neutral pH.

Examples of alkylaryl groups are methylphenyl (tolyl), ethylphenyl, 4-isopropylphenyl, and xylyl groups. Examples of arylalkyl (aralkyl) groups are benzyl, phenylethyl and trityl groups. Examples of alkylarylalkyl groups are methylbenzyl and 4-isopropyl benzyl groups. Carboxyarylalkyl moieties may for example be radicals derived from o, m or p-methyl benzoic acid, or o-, m- or p-ethyl benzoic acid. Carboxyalkylarylalkyl moieties may for example be o-, m- or p-methyl phenylacetic acid. Carboxyalkenylarylalkyl moieties may for example be or from o-, m- or p-methyl cinnamic acid.

Multiple carboxylated substituents such as those being or comprising carboxyalkyl groups present on the polyamine derivative may be the same or different. For simplicity, they may be the same. The carboxy group of the carboxylated substituent may be bound to any carbon atom of the hydrophobic linker. Preferably, the carboxy group is bound to a carbon atom as follows: if z is the number of carbon atoms in the longest carbon chain in the carboxylated substituent (such as the carboxyalkyl group) to the carbon atom that is bound to a polyamine nitrogen atom, the carboxy group is bound to a carbon atom at a position that is more than z/2 atom positions away from the polyamine nitrogen, if the carbon atom bound to the polyamine nitrogen is counted as position 1. If the value of z/2 is not an integer, the above definition leads to the position defined by the next integer>z/2. In one embodiment, the carboxy group is bound to the carbon atom of the hydrophobic linker that is most remote (in terms of the number of carbon atoms) from the polyamine nitrogen atom to which the hydrophobic linker (alkylene chain in the case of carboxyalkyl groups) is connected. The carboxy group may be bound to the carbon atom that is farthest away from the polyamine nitrogen within the carboxylated substituent (or carboxyalkyl group), such as to the terminal (omega position) carbon atom of the carboxylated substituents (or carboxyalkyl group) in case of a linear carboxylated substituent.

In some embodiments, the hydrophobic substituents comprise from 2 to 40 carbon atoms, in some embodiments, from 3 to 40 carbon atoms, in some embodiments from 6 to 40 carbon atoms and in some embodiments from 6 to 20 carbon atoms. The hydrophobic substituents may comprise from 1 to 3, preferably 1 or 2, heteroatoms selected from O, N, and S, provided said hydrophobic substituents comprise 6 or more carbon atoms. Preferably, the heteroatoms are selected from O and S. Thus, the hydrophobic substituents may be hydrocarbyl groups or heterohydrocarbyl groups, the latter comprising from 1 to 3 heteroatoms as mentioned before. Among the plurality of hydrophobic substituents of a molecule of said polyamine derivative, there may be exclusively hydrocarbyl groups, exclusively heterohydrocarbyl groups, or there may be hydrocarbyl groups and heterohydrocarbyl groups. In some embodiments, the plurality of hydrophobic substituents are all hydrocarbyl groups. In some embodiments, the plurality of hydrophobic substituents are all heterohydrocarbyl groups.

Where the hydrophobic substituents are hydrocarbyl groups, they may be selected from alkyl groups, alkenyl groups, alkynyl groups, cycloalkyl groups, cycloalkenyl groups, cycloalkylalkyl groups, alkylcycloalkyl groups, alkylcycloalkylalkyl groups, aryl groups, alkylaryl groups, arylalkyl groups, and alkylarylalkyl groups and groups comprising two or more groups from the aforementioned list. Provided the hydrophobic substituent comprises 6 or more carbon atoms, it is possible to replace 1, 2 or 3 of the carbon atoms of said hydrocarbyl groups by oxygen, nitrogen or sulfur, preferably oxygen or sulfur, thereby forming heterohydrocarbyl substituents. Such heterohydrocarbyl substituents may comprise functional groups selected from —O—, —S—, —N(H) C(O)—, —C(O)O—, —OC(O)N(H)—, —C(O)—, —C(O)—N(H)—, —N(H)—C(O)—O—, —O—C(O)—, or —S—S—.

In some embodiments, the hydrophobic substituents are or comprise alkyl groups such as linear or branched alkyl groups, or cycloalkyl groups. Alkyl groups may be n-alkyl or isoalkyl groups. Examples of alkyl groups are propyl, butyl, pentyl, hexyl, heptyl, octyl, nonyl, decyl, undecyl, dodecyl, tetradecyl or hexadecyl groups. Examples of cycloalkyl groups are cyclopentyl, cyclohexyl and cycloheptyl groups.

Examples of alkenyl groups are propenyl, butenyl, pentenyl, hexenyl, heptenyl, octenyl, nonenyl, decenyl, undecenyl, dodecenyl, tetradecenyl and hexadecenyl groups. Examples of alkynyl groups are propynyl, butynyl, pentynyl, hexynyl, heptynyl, octynyl, nonynyl, decynyl, undecynyl, dodecynyl, tetradecynyl and hexadecynyl groups.

Examples of cycloalkenyl groups are cyclopentenyl, cyclohexenyl and cycloheptenyl groups. Cycloalkylalkyl groups are groups wherein a cycloalkyl group is linked to an alkylene group corresponding to an alkyl group. Examples are cyclopentylmethyl, cyclopentylethyl, cyclohexylmethyl, cyclohexylethyl etc.

Alkylcycloalkyl groups are groups wherein an alkyl group is linked to a cycloalkylene group corresponding to a cycloalkyl group. Examples of alkylcycloalkyl groups are methylcyclopentyl, ethylcyclopentyl, propylcyclopentyl, butylcyclopentyl, pentylcyclopentyl, hexylcylopentyl, methylcyclohexyl, ethylcyclohexyl, propylcyclohexyl, butylcyclohexyl, pentylcyclohexyl and hexylcylohexyl.

Alkylcycloalkylalkyl groups are groups wherein an alkyl group is linked to a cycloalkylalkylene group.

In some embodiments, the hydrophobic substituent comprises an aryl group and has from 6 to 20, preferably from 7 to 15 carbon atoms. Aryl groups include aromatic hydrocarbyl groups (carbon-only aryl groups) and aromatic heterohydrocarbyl groups (heteroaryl groups). Examples of the former are phenyl, naphthyl and phenanthryl. In some embodiments, nitrogen-containing heteroaryl groups have a pK value of <5 for avoiding additional cationic charges at neutral pH. Examples of such nitrogen-containing heteroaryl groups are indolyl groups pyrazinyl groups, pyridazinyl groups, pyrimidinyl groups, cinnolinyl groups, phthalazinyl groups and purinyl groups. In some embodiments, oxygen-containing heterohydrocarbyl groups that form hydroxy groups have a pK>12 for avoiding negative charges at neutral pH.

Examples of alkylaryl groups are methylphenyl (tolyl), ethylphenyl, 4-isopropylphenyl, methylindolyl and xylyl groups. Examples of arylalkyl (aralkyl) groups are benzyl, phenylethyl, indolylmethyl and trityl groups. Examples of alkylarylalkyl groups are methylbenzyl and 4-isopropylbenzyl groups.

Different hydrophobic substituents on a molecule of the polyamine derivative may be the same or may be different. For simplicity, they may be the same.

In some embodiments, the polyamine derivative has a linear polyethylenimine moiety of from 2 to 500 kDa (in terms of number average molecular weight), the carboxylated substituents have from 10 to 16 carbon atoms and are n-alkylcarboxylic acids and the hydrophobic substituents have from 1 to 12 carbon atoms and are alkyls, preferably n-alkyls, and/or alkylarylalkyls.

In some embodiments, the polyamine derivative has a branched polyethylenimine moiety of from 0.5 to 200 kDa (in terms of number average molecular weight), the carboxylated substituents have from 10 to 16 carbon atoms and are n-alkylcarboxylic acids and the hydrophobic substituents have from 1 to 12 carbon atoms and are alkyls, preferably n-alkyls, and/or alkylarylalkyls.

In some embodiments, a polyamine derivative is:

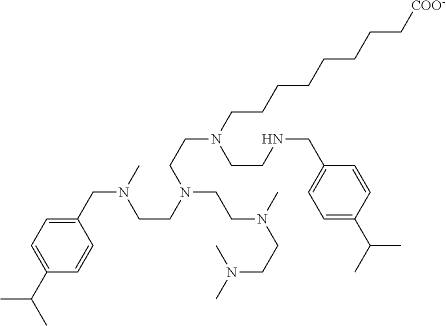

or a pharmaceutically acceptable salt thereof.

In some embodiments, a complex comprises a cationic polymer, an anionic polymer, RNA, and a polyamine, wherein the polyamine is:

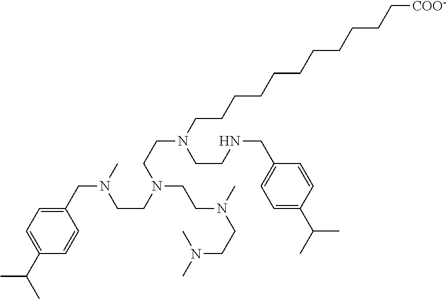

or a pharmaceutically acceptable salt thereof.

### RNA

In some embodiments, a complex described herein comprises a cationic polymer, an anionic polymer, and a nucleic acid. In some embodiments, a nucleic acid is RNA. In some embodiments, RNA is monomeric RNA (i.e., a complex described herein comprises a monomeric RNA molecule). “Monomeric RNA,” as used herein, refers to RNA that is an individual RNA molecule, and not an aggregate, or as a dimer, trimer, or oligomer of RNA. Therefore, reference to a complex comprising monomeric RNA (e.g., a monomeric RNA molecule) is understood to be a complex that with a single RNA molecule that is not aggregate, or as a dimer, trimer, or oligomer of RNA. A person of skill in the art will understand how to evaluate the presence of monomeric RNA in a sample. For example by a centrifugation and quantification method. That is, in some embodiments, the presence and quantification of monomeric RNA in a sample is determined by centrifugation of a sample comprising RNA, extraction of the supernatant, followed by measuring the concentration of RNA in the supernatant relative to the total amount of RNA in the sample. In some embodiments, the presence and evaluation of monomeric RNA in a sample is by an ultracentrifugation assay, where samples are measured with analytic ultracentrifuge and light absorption is measured at two different wavelengths: 255 nm and 650 nm. In some embodiments, such samples are centrifuged at 80,000×g at room temperature.

In some embodiments, an RNA amenable to technologies described herein is a single-stranded RNA. In some embodiments, an RNA as disclosed herein is a linear RNA. In some embodiments, a single-stranded RNA is a non-coding RNA in that its nucleotide sequence does not include an open reading frame (or complement thereof). In some embodiments, a single-stranded RNA has a nucleotide sequence that encodes (or is the complement of a sequence that encodes) a polypeptide or a plurality of polypeptides (e.g., epitopes) of the present disclosure.

In some embodiments, an RNA is or comprises an siRNA, an miRNA, or other non-coding RNA.

In many embodiments, a relevant RNA includes at least one open reading frame (ORF) (e.g., is an mRNA); in some embodiments, a relevant RNA includes a single ORF; in some embodiments, a relevant RNA includes more than one ORF.

In some embodiments, an RNA comprises an ORF, e.g., encoding a polypeptide of interest or encoding a plurality of polypeptides of interest. In some embodiments, an RNA produced in accordance with technologies provided herein comprises a plurality of ORFs (e.g., encoding a plurality of polypeptides). In some embodiments, an RNA produced in accordance with technologies herein comprises a single ORF that encodes a plurality of polypeptides. In some such embodiments, polypeptides are or comprise antigens or epitopes thereof (e.g., relevant antigens).

In some embodiments, an ORF for use in accordance with the present disclosure encodes a polypeptide that includes a signal sequence, e.g., that is functional in mammalian cells, such as an intrinsic signal sequence or a heterologous signal sequence. In some embodiments, a signal sequence directs secretion of an encoded polypeptide, in some embodiments, a signal sequence directs transport of an encoded polypeptide into a defined cellular compartment, preferably the cell surface, the endoplasmic reticulum (ER) or the endosomal-lysosomal compartment.

In some embodiments, an ORF encodes a polypeptide that includes a multimerization element (e.g., an intrinsic or heterologous multimerization element). In some embodiments, an ORF that encodes a surface polypeptide (e.g., that includes a signal sequence directing surface localization) includes a multimerization element.

In some embodiments, an ORF encodes a polypeptide that includes a transmembrane element or domain.

In some embodiments, an ORF is codon-optimized for expression in a cells of a particular host, e.g., a mammalian host, e.g., a human.

In some embodiments, an RNA includes unmodified uridine residues; an RNA that includes only unmodified uridine residues may be referred to as a “uRNA”. In some embodiments, an RNA includes one or more modified uridine residues; in some embodiments, such an RNA (e.g., an RNA including entirely modified uridine residues) is referred to as a “modRNA”. In some embodiments, an RNA may be a self-amplifying RNA (saRNA). In some embodiments, an RNA may be a trans-amplifying RNA (see, for example, WO2017/162461).

In some embodiments, a relevant RNA includes a polypeptide-encoding portion or a plurality of polypeptide-encoding portions. In some particular embodiments, such a portion or portions may encode a polypeptide or polypeptides that is or comprises a biologically active polypeptide or portion thereof (e.g., an enzyme or cytokine or therapeutic protein such as a replacement protein or antibody or portion thereof). In some particular embodiments, such a portion or portions may encode a polypeptide or polypeptides that is or comprises an antigen (or an epitope thereof), a cytokine, an enzyme, etc. In some embodiments, an encoded polypeptide or polypeptides may be or include one or more neoantigens or neoepitopes associated with a tumor. In some embodiments, an encoded polypeptide or polypeptides may be or include one or more antigens (or epitopes thereof) of an infectious agent (e.g., a bacterium, fungus, virus, etc.). In certain embodiments, an encoded polypeptide may be a variant of a wild type polypeptide.

In some embodiments, a single-stranded RNA (e.g., mRNA) may comprise a secretion signal-encoding region (e.g., a secretion signal-encoding region that allows an encoded target entity or entities to be secreted upon translation by cells). In some embodiments, such a secretion signal-encoding region may be or comprise a non-human secretion signal. In some embodiments, such a secretion signal-encoding region may be or comprise a human secretion signal.

In some embodiments, a single-stranded RNA (e.g., mRNA) may comprise at least one non-coding element (e.g., to enhance RNA stability and/or translation efficiency). Examples of non-coding elements include but are not limited to a 3′ untranslated region (UTR), a 5′ UTR, a cap structure (e.g., in some embodiments, an enzymatically-added cap; in some embodiments, a co-transcriptional cap), a poly adenine (polyA) tail (e.g., that, in some embodiments, may be or comprise 100 A residues or more, and/or in some embodiments may include one or more “interrupting” [i.e., non-A] sequence elements), and any combinations thereof. Exemplary embodiments of such non-coding elements may be found, for example, in WO2011015347, WO2017053297, U.S. Pat. Nos. 10,519,189, 10,494,399, WO2007024708, WO2007036366, WO2017060314, WO2016005324, WO2005038030, WO2017036889, WO2017162266, and WO2017162461, each of which is incorporated herein by referenced in its entirety.

### Formats

At least four formats useful for RNA pharmaceutical compositions (e.g., immunogenic compositions or vaccines) have been developed, namely non-modified uridine containing mRNA (uRNA), nucleoside modified mRNA (modRNA), self-amplifying mRNA (saRNA), and trans-amplifying RNAs.

Features of a non-modified uridine platform may include, for example, one or more of intrinsic adjuvant effect, good tolerability and safety, and strong antibody and T cell responses.

Features of modified uridine (e.g., pseudouridine) platform may include reduced adjuvant effect, blunted immune innate immune sensor activating capacity and thus augmented antigen expression, good tolerability and safety, and strong antibody and CD4-T cell responses. As noted herein, the present disclosure provides an insight that such strong antibody and CD4 T cell responses may be particularly useful for vaccination.

Features of self-amplifying platform may include, for example, long duration of polypeptide (e.g., protein) expression, good tolerability and safety, higher likelihood for efficacy with very low vaccine dose.

In some embodiments, a self-amplifying platform (e.g., RNA) comprises two nucleic acid molecules, wherein one nucleic acid molecule encodes a replicase (e.g., a viral replicase) and the other nucleic acid molecule is capable of being replicated (e.g., a replicon) by said replicase in trans (trans-replication system). In some embodiments, a self-amplifying platform (e.g., RNA) comprises a plurality of nucleic acid molecules, wherein said nucleic acids encode a plurality of replicases and/or replicons.

In some embodiments, a trans-replication system comprises the presence of both nucleic acid molecules in a single host cell.

In some such embodiments, a nucleic acid encoding a replicase (e.g., a viral replicase) is not capable of self-replication in a target cell and/or target organism. In some such embodiments, a nucleic acid encoding a replicase (e.g., a viral replicase) lacks at least one conserved sequence element important for (−) strand synthesis based on a (+) strand template and/or for (+) strand synthesis based on a (−) strand template.

In some embodiments, a self-amplifying RNA comprises a 5′-cap; in some trans-replication systems, at least an RNA encoding a replicase is capped. Without wishing to be bound by any one theory, it has been found that a 5′-cap can be important for high level expression of a gene of interest in trans.

In some embodiments, a self-amplifying platform does not require propagation of virus particles (e.g., is not associated with undesired virus-particle formation). In some embodiments, a self-amplifying platform is not capable of forming virus particles. In some embodiments, an RNA may comprise an Internal Ribosomal Entry Site (IRES) element. In some embodiments, an RNA does not comprise an IRES site; in particular, in some embodiments, an saRNA does not comprise an IRES site. In some such embodiments, translation of a gene of interest and/or replicase is not driven by an IRES element. In some embodiments, an IRES element is substituted by a 5′-cap. In some such embodiments, substitution by a 5′-cap does not affect the sequence of a polypeptide encoded by an RNA.

In some embodiments, a complex described herein comprises mRNA, modRNA or saRNA. In some embodiments, a complex comprises mRNA. In some embodiments, a complex comprises modRNA. In some embodiments, a complex comprises saRNA.

### Anionic Polymers

As described generally herein, a complex of the present disclosure comprises an anionic polymer capable of encapsulating a core complex also described herein. An anionic polymer described herein can be linear or branched, and comprises one or more anionic moieties or groups. As used herein, an “anionic” moiety or group is a group that is negatively charged at neutral pH (e.g., about a pH of 7), or is otherwise capable of becoming negatively charged due to a change in pH, such as to physiological pH (e.g., a pH of 7.4). In some embodiments, an anionic polymer is a polyanionic polymer, e.g., a polymer having one or more anionic groups. In some embodiments, an anionic group is a —CO₂⁻, a —OSO₃⁻, or a —OPO₃²⁻ group.

In some embodiments, an anionic polymer is selected from poly-L-glutamic acid, a poly-L-aspartic acid, a polysaccharide, a derivative of poly(2-oxazoline), a derivative of poly(2-oxazine), poly(2-methoxycarbonylethyl-2-oxazoline), poly(2-methoxycarbonylpropyl-2-oxazoline), poly(2-methoxycarbonylethyl-2-oxazine), poly(2-methoxycarbonylpropyl-2-oxazine), and polyphosphate.

In some embodiments, an anionic polymer is selected from poly-L-glutamic acid, a poly-L-aspartic acid, a polysaccharide, poly(2-methoxycarbonylethyl-2-oxazoline), poly(2-methoxycarbonylpropyl-2-oxazoline), poly(2-methoxycarbonylethyl-2-oxazine), poly(2-methoxycarbonylpropyl-2-oxazine), and polyphosphate.

In some embodiments, an anionic polymer is a polyglutamic acid. In some embodiments, an anionic polymer is poly-L-glutamic acid. In some embodiments, an anionic polymer is fondaparinux, represented by the structure below:

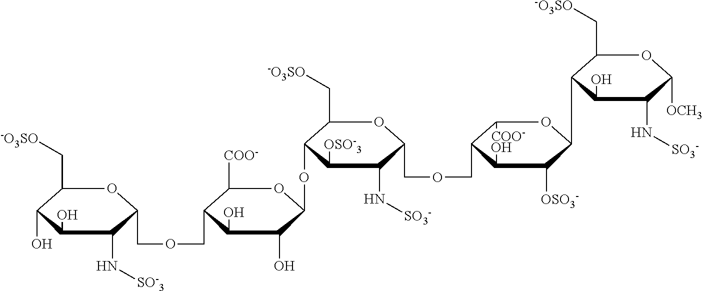

In some embodiments, an anionic polymer is a derivative of poly(2-oxazoline). In some embodiments, an anionic polymer is a derivative of poly(2-oxazine). In some embodiments, an anionic polymer is poly(2-methoxycarbonylethyl-2-oxazoline) (pC2MestOx). In some embodiments, an anionic polymer is poly(2-methoxycarbonylpropyl-2-oxazoline) (pC3MestOx). In some embodiments, an anionic polymer is poly(2-methoxycarbonylethyl-2-oxazine) (pC2MestOz). In some embodiments, an anionic polymer is poly(2-methoxycarbonylpropyl-2-oxazine) (pC3MestOz). In some embodiments, an anionic polymer is a polyphosphate (PolyP). In some embodiments, an anionic polymer is a homopolymer. In some embodiments, an anionic polymer is a homopolymer comprising about 10 to about 150 repeating monomeric units. In some embodiments, an anionic polymer is a homopolymer comprising about 10 to about 100 repeating monomeric units. In some embodiments, an anionic polymer is a homopolymer comprising about 20 to about 100 repeating monomeric units. In some embodiments, an anionic polymer is a homopolymer comprising about 50 to about 150 repeating monomeric units. In some embodiments, an anionic polymer is a homopolymer comprising about 50 repeating monomeric units. In some embodiments, an anionic polymer is a homopolymer comprising about 100 repeating monomeric units.

In some embodiments, an anionic polymer that is a homopolymer is poly-L-glutamic acid having from about 10 to about 150 repeating units of glutamic acid. In some embodiments, an anionic polymer that is a homopolymer is poly-L-glutamic acid having from about 20 to about 100 repeating units of glumatic acid. In some embodiments, an anionic polymer that is a homopolymer is poly-L-glutamic acid, having from about 50 to 150 repeating units of glutamic acid. In some embodiments, an anionic polymer that is a homopolymer is poly-L-glutamic acid, having about 50 repeating units of glutamic acid. In some embodiments, an anionic polymer that is a homopolymer is poly-L-glutamic acid, having about 100 repeating units of glutamic acid.

In some embodiments, an anionic polymer that is a homopolymer is poly-L-aspartic acid having from about 10 to about 150 repeating units of aspartic acid. In some embodiments, an anionic polymer that is a homopolymer is poly-L-aspartic acid having from about 20 to about 100 repeating units of glum aspartic acid. In some embodiments, an anionic polymer that is a homopolymer is poly-L-aspartic acid, having from about 50 to 150 repeating units of aspartic acid. In some embodiments, an anionic polymer that is a homopolymer is poly-L-aspartic acid, having about 50 repeating units of aspartic acid. In some embodiments, an anionic polymer that is a homopolymer is poly-L-aspartic acid, having about 100 repeating units of aspartic acid.

In some embodiments, an anionic polymer that is a homopolymer is a polyphosphate having from about 10 to about 150 repeating units of phosphates. In some embodiments, an anionic polymer that is a homopolymer is a polyphosphate having from about 20 to about 100 repeating units of phosphates. In some embodiments, an anionic polymer that is a homopolymer is a polyphosphate having from about 50 to about 150 repeating units of phosphates. In some embodiments, an anionic polymer that is a homopolymer is a polyphosphate having from about 50 repeating units of phosphates.

In some embodiments, an anionic polymer that is a homopolymer is a derivative of poly(2-oxazoline) having from about 10 to about 150 repeating units of a derivative of 2-oxazoline. In some embodiments, an anionic polymer that is a homopolymer is a derivative of poly(2-oxazoline) having from about 20 to about 100 repeating units of a derivative of 2-oxazoline. In some embodiments, an anionic polymer that is a homopolymer is a derivative of poly(2-oxazoline), having from about 50 to 150 repeating units of a derivative of 2-oxazoline. In some embodiments, an anionic polymer that is a homopolymer is a derivative of poly(2-oxazoline), having about 50 repeating units of a derivative of 2-oxazoline. In some embodiments, an anionic polymer that is a homopolymer is a derivative of poly(2-oxazoline), having about 100 repeating units of a derivative of 2-oxazoline.

In some embodiments, an anionic polymer that is a homopolymer is a derivative of poly(2-oxazine) having from about 10 to about 150 repeating units of a derivative of 2-oxazine. In some embodiments, an anionic polymer that is a homopolymer is a derivative of poly(2-oxazine) having from about 20 to about 100 repeating units of a derivative of 2-oxazine. In some embodiments, an anionic polymer that is a homopolymer is a derivative of poly(2-oxazine), having from about 50 to 150 repeating units of a derivative of 2-oxazine. In some embodiments, an anionic polymer that is a homopolymer is a derivative of poly(2-oxazine), having about 50 repeating units of a derivative of 2-oxazine. In some embodiments, an anionic polymer that is a homopolymer is a derivative of poly(2-oxazine), having about 100 repeating units of a derivative of 2-oxazine.

In some embodiments, an anionic polymer that is a homopolymer is poly(2-methoxycarbonylethyl-2-oxazoline) having from about 10 to about 150 repeating units of 2-methoxycarbonylethyl-2-oxazoline. In some embodiments, an anionic polymer that is a homopolymer is poly(2-methoxycarbonylethyl-2-oxazoline) having from about 20 to about 100 repeating units of 2-methoxycarbonylethyl-2-oxazoline. In some embodiments, an anionic polymer that is a homopolymer is poly(2-methoxycarbonylethyl-2-oxazoline), having from about 50 to 150 repeating units of 2-methoxycarbonylethyl-2-oxazoline. In some embodiments, an anionic polymer that is a homopolymer is poly(2-methoxycarbonylethyl-2-oxazoline), having about 50 repeating units of 2-methoxycarbonylethyl-2-oxazoline. In some embodiments, an anionic polymer that is a homopolymer is poly(2-methoxycarbonylethyl-2-oxazoline), having about 100 repeating units of 2-methoxycarbonylethyl-2-oxazoline.

In some embodiments, an anionic polymer that is a homopolymer is poly(2-methoxycarbonylpropyl-2-oxazoline) having from about 10 to about 150 repeating units of 2-methoxycarbonylpropyl-2-oxazoline. In some embodiments, an anionic polymer that is a homopolymer is poly(2-methoxycarbonylpropyl-2-oxazoline) having from about 20 to about 100 repeating units of 2-methoxycarbonylpropyl-2-oxazoline. In some embodiments, an anionic polymer that is a homopolymer is poly(2-methoxycarbonylpropyl-2-oxazoline), having from about 50 to 150 repeating units of 2-methoxycarbonylpropyl-2-oxazoline. In some embodiments, an anionic polymer that is a homopolymer is poly(2-methoxycarbonylpropyl-2-oxazoline), having about 50 repeating units of 2-methoxycarbonylpropyl-2-oxazoline. In some embodiments, an anionic polymer that is a homopolymer is poly(2-methoxycarbonylpropyl-2-oxazoline), having about 100 repeating units of 2-methoxycarbonylpropyl-2-oxazoline.

In some embodiments, an anionic polymer that is a homopolymer is poly(2-methoxycarbonylethyl-2-oxazine) having from about 10 to about 150 repeating units of 2-methoxycarbonylethyl-2-oxazine. In some embodiments, an anionic polymer that is a homopolymer is poly(2-methoxycarbonylethyl-2-oxazine) having from about 20 to about 100 repeating units of 2-methoxycarbonylethyl-2-oxazine. In some embodiments, an anionic polymer that is a homopolymer is poly(2-methoxycarbonylethyl-2-oxazine), having from about 50 to 150 repeating units of 2-methoxycarbonylethyl-2-oxazine. In some embodiments, an anionic polymer that is a homopolymer is poly(2-methoxycarbonylethyl-2-oxazine), having about 50 repeating units of 2-methoxycarbonylethyl-2-oxazine. In some embodiments, an anionic polymer that is a homopolymer is poly(2-methoxycarbonylethyl-2-oxazine), having about 100 repeating units of 2-methoxycarbonylethyl-2-oxazine.

In some embodiments, an anionic polymer that is a homopolymer is poly(2-methoxycarbonylpropyl-2-oxazine) having from about 10 to about 150 repeating units of 2-methoxycarbonylpropyl-2-oxazine. In some embodiments, an anionic polymer that is a homopolymer is poly(2-methoxycarbonylpropyl-2-oxazine) having from about 20 to about 100 repeating units of 2-methoxycarbonylpropyl-2-oxazine. In some embodiments, an anionic polymer that is a homopolymer is poly(2-methoxycarbonylpropyl-2-oxazine), having from about 50 to 150 repeating units of 2-methoxycarbonylpropyl-2-oxazine. In some embodiments, an anionic polymer that is a homopolymer is poly(2-methoxycarbonylpropyl-2-oxazine), having about 50 repeating units of 2-methoxycarbonylpropyl-2-oxazine. In some embodiments, an anionic polymer that is a homopolymer is poly(2-methoxycarbonylpropyl-2-oxazine), having about 100 repeating units of 2-methoxycarbonylpropyl-2-oxazine.

In some embodiments, an anionic polymer is a heteropolymer. In some embodiments, an anionic polymer is a heteropolymer comprising monomers of glutamic acid, ethylene glycol, propylene glycol, sarcosine, phosphates, 2-methyl-2-oxazoline, methoxycarbonylethyl-2-oxazoline, 2-ethyl-2-oxazoline, 2-methyl-2-oxazine, 2-methoxycarbonylethyl-2-oxazine, 2-methoxycarbonylpropyl-2-oxazoline, 2-methoxycarbonylpropyl-2-oxazine, 2-(2-(2-aminoethoxy) ethoxy) acetic acid, and/or 2-(2-(2-methylaminoethoxy) ethoxy) acetic acid. In some embodiments, an anionic polymer is a heteropolymer comprising monomers of glutamic acid and ethylene glycol. In some embodiments, an anionic polymer is a heteropolymer comprising monomers of glutamic acid and propylene glycol. In some embodiments, an anionic polymer is a heteropolymer comprising monomers of glutamic acid and sarcosine. In some embodiments, an anionic polymer is a heteropolymer comprising monomers of glutamic acid and 2-methoxycarbonylethyl-2-oxazoline.

In some embodiments, an anionic polymer is a block-co-polymer. In some embodiments, an anionic polymer is a block-co-polymer comprising blocks of polymers selected from the group consisting of poly(2-methoxycarbonylethyl-2-oxazoline), poly(2-methyl-2-oxazoline), poly-L-glutamic acid, poly-L-aspartic acid, polyphosphates, poly(N-methyl-sarcosine), poly(ethylene glycol), poly(propylene glycol), poly(2-methoxycarbonylpropyl-2-oxazoline), poly(2-methoxycarbonylethyl-2-oxazine), poly(2-methoxycarbonylpropyl-2-oxazine) poly-2-(2-(2-aminoethoxy) ethoxy) acetic acid, and poly-2-(2-(2-methylaminoethoxy) ethoxy) acetic acid.

In some embodiments, a block-co-polymer comprises about 10 to about 100 monomeric repeating units of each block. In some embodiments, a block-co-polymer comprises about 20 to about 100 monomeric repeating units of each block. In some embodiments, a block-co-polymer comprises about 50 monomeric repeating units of each block. In some embodiments, a block-co-polymer comprises an anionic block and a stealth block. In some embodiments, a block-co-polymer comprises an anionic block, wherein the anionic block is selected from poly(2-methoxycarbonylethyl-2-oxazoline), poly-L-glutamic acid, poly-L-aspartic acid, poly(2-methoxycarbonylpropyl-2-oxazoline), poly(2-methoxycarbonylethyl-2-oxazine), and poly(2-methoxycarbonylpropyl-2-oxazoline); and the stealth block is selected from poly(ethylene glycol), poly(propylene glycol), poly(2-methyl-2-oxazoline), poly(N-methyl-sarcosine), poly-2-(2-(2-aminoethoxy) ethoxy) acetic acid (AEEA), and poly-2-(2-(2-methylaminoethoxy) ethoxy) acetic acid. In some embodiments, a block-co-polymer comprises from about 10 to about 100 repeating monomeric units of the anionic block, and from about 10 to about 100 repeating monomeric units of the stealth block.

In some embodiments, a block-co-polymer comprises a first anionic block and a second anionic block. In some embodiments, a first anionic block and a second anionic block are not the same. In some embodiments, a block-co-polymer comprises a first anionic block, wherein the first anionic block is selected from poly(2-methoxycarbonylethyl-2-oxazoline), poly-L-glutamic acid, poly-L-aspartic acid, poly(2-methoxycarbonylpropyl-2-oxazoline), poly(2-methoxycarbonylethyl-2-oxazine), and poly(2-methoxycarbonylpropyl-2-oxazine); and the second anionic block is selected from poly(2-methoxycarbonylethyl-2-oxazoline), poly-L-glutamic acid, poly-L-aspartic acid, poly(2-methoxycarbonylpropyl-2-oxazoline), poly(2-methoxycarbonylethyl-2-oxazine), and poly(2-methoxycarbonylpropyl-2-oxazine). In some embodiments, a block-co-polymer comprises from about 10 to about 100 repeating monomeric units of the first anionic block, and from about 10 to about 100 repeating monomeric units of the second anionic block.

In some embodiments, a block-co-polymer comprises three or more anionic blocks, wherein each block comprises an anionic polymer independently selected from poly(2-methoxycarbonylethyl-2-oxazoline), poly-L-glutamic acid, poly-L-aspartic acid, poly(2-methoxycarbonylpropyl-2-oxazoline), poly(2-methoxycarbonylethyl-2-oxazine), and poly(2-methoxycarbonylpropyl-2-oxazine). In some embodiments, each anionic block of the block-co-polymer comprises from about 10 to about 100 monomeric units of the anionic polymer.

In some embodiments, an anionic polymer that is a block-co-polymer is poly-L-glutamic acid-block-poly(2-methoxy-2-oxazoline). In some embodiments, an anionic polymer that is a block-co-polymer is poly-L-glutamic acid-block-poly(N-methyl-sarcosine). In some embodiments, an anionic polymer that is a block-co-polymer is poly-L-glutamic acid-block-poly(ethylene glycol). In some embodiments, an anionic polymer that is a block-co-polymer is poly-L-glutamic acid-block-poly(propylene glycol). In some embodiments, an anionic polymer that is a block-co-polymer is poly-L-glutamic acid-block-poly-2-(2-(2-aminoethoxy) ethoxy) acetic acid. In some embodiments, an anionic polymer that is a block-co-polymer is poly-L-glutamic acid-block-poly-2-(2-(2-In some embodiments, an anionic polymer that is a block-co-polymer is poly-L-aspartic acid-block-poly(2-methoxy-2-oxazoline). In some embodiments, an anionic polymer that is a block-co-polymer is poly-L-aspartic acid-block-poly(N-methyl-sarcosine). In some embodiments, an anionic polymer that is a block-co-polymer is poly-L-aspartic acid-block-poly(ethylene glycol). In some embodiments, an anionic polymer that is a block-co-polymer is poly-L-aspartic acid-block-poly(propylene glycol). In some embodiments, an anionic polymer that is a block-co-polymer is poly-L-aspartic acid-block-poly-2-(2-(2-aminoethoxy)ethoxy)acetic acid. In some embodiments, an anionic polymer that is a block-co-polymer is poly-L-aspartic acid-block-poly-2-(2-(2-methylaminoethoxy)ethoxy)acetic acid.

In some embodiments, an anionic polymer that is a block-co-polymer is poly(2-methoxycarbonylethyl-2-oxazoline)-block-poly(2-methyl-2-oxazoline). In some embodiments, an anionic polymer that is a block-co-polymer is poly(2-methoxycarbonylethyl-2-oxazoline)-block-poly(N-methyl-sarcosine). In some embodiments, an anionic polymer that is a block-co-polymer is poly(2-methoxycarbonylethyl-2-oxazoline)-block-poly(ethylene glycol). In some embodiments, an anionic polymer that is a block-co-polymer is poly(2-methoxycarbonylethyl-2-oxazoline)-block-poly(propylene glycol). In some embodiments, an anionic polymer that is a block-co-polymer is poly(2-methoxycarbonylethyl-2-oxazoline)-block-poly-2-(2-(2-aminoethoxy)ethoxy)acetic acid. In some embodiments, an anionic polymer that is a block-co-polymer is poly(2-methoxycarbonylethyl-2-oxazoline)-block-poly-2-(2-(2-methylaminoethoxy)ethoxy)acetic acid.

In some embodiments, an anionic polymer that is a block-co-polymer is poly(2-methoxycarbonylpropyl-2-oxazoline)-block-poly(2-methyl-2-oxazoline). In some embodiments, an anionic polymer that is a block-co-polymer is poly(2-methoxycarbonylpropyl-2-oxazoline)-block-poly(N-methyl-sarcosine). In some embodiments, an anionic polymer that is a block-co-polymer is poly(2-methoxycarbonylpropyl-2-oxazoline)-block-poly(ethylene glycol). In some embodiments, an anionic polymer that is a block-co-polymer is poly(2-methoxycarbonylpropyl-2-oxazoline)-block-poly(propylene glycol). In some embodiments, an anionic polymer that is a block-co-polymer is poly(2-methoxycarbonylpropyl-2-oxazoline)-block-poly-2-(2-(2-aminoethoxy)ethoxy)acetic acid. In some embodiments, an anionic polymer that is a block-co-polymer is poly(2-methoxycarbonylpropyl-2-oxazoline)-block-poly-2-(2-(2-methylaminoethoxy)ethoxy)acetic acid.

In some embodiments, an anionic polymer that is a block-co-polymer is poly(2-methoxycarbonylethyl-2-oxazine)-block-poly(2-methyl-2-oxazoline). In some embodiments, an anionic polymer that is a block-co-polymer is poly(2-methoxycarbonylethyl-2-oxazine)-block-poly(N-methyl-sarcosine). In some embodiments, an anionic polymer that is a block-co-polymer is poly(2-methoxycarbonylethyl-2-oxazine)-block-poly(ethylene glycol). In some embodiments, an anionic polymer that is a block-co-polymer is poly(2-methoxycarbonylethyl-2-oxazine)-block-poly(propylene glycol). In some embodiments, an anionic polymer that is a block-co-polymer is poly(2-methoxycarbonylethyl-2-oxazine)-block-poly-2-(2-(2-aminoethoxy)ethoxy)acetic acid. In some embodiments, an anionic polymer that is a block-co-polymer is poly(2-methoxycarbonylethyl-2-oxazine)-block-poly-2-(2-(2-methylaminoethoxy)ethoxy)acetic acid.

In some embodiments, an anionic polymer that is a block-co-polymer is poly(2-methoxycarbonylpropyl-2-oxazine)-block-poly(2-methyl-2-oxazoline). In some embodiments, an anionic polymer that is a block-co-polymer is poly(2-methoxycarbonylpropyl-2-oxazine)-block-poly(N-methyl-sarcosine). In some embodiments, an anionic polymer that is a block-co-polymer is poly(2-methoxycarbonylpropyl-2-oxazine)-block-poly(ethylene glycol). In some embodiments, an anionic polymer that is a block-co-polymer is poly(2-methoxycarbonylpropyl-2-oxazine)-block-poly(propylene glycol). In some embodiments, an anionic polymer that is a block-co-polymer is poly(2-methoxycarbonylpropyl-2-oxazine)-block-poly-2-(2-(2-aminoethoxy)ethoxy)acetic acid. In some embodiments, an anionic polymer that is a block-co-polymer is poly(2-methoxycarbonylpropyl-2-oxazine)-block-poly-2-(2-(2-methylaminoethoxy)ethoxy)acetic acid.

In some embodiments, an anionic polymer is a homopolymer poly-α-L-glutamic acid having 10, 20, 50, or 100 repeating units (PGA(10), (20), (50), and (100), respectively). In some embodiments, an anionic polymer is homopolymer poly-L-α-L-glutamic acid having 10, 20, 50, or 100 repeating units (PGA(10), (20), (50), and (100), respectively).

In some embodiments, an anionic polymer is a homopolymer of n-butyl-poly-(L-aspartic acid) having 10, 20, 50, or 100 repeating units (PAsp (10), (20), (50), and (100), respectively).

In some embodiments, an anionic polymer is a block-copolymer of poly-α-L-glutamic acid (PGA)-block-poly-N-methyl-sarcosine (pSar) having 10, 20, 50, or 100 units of PGA and about 10, 25, or 50 units of pSar (PGA(10, 20, 50 or 100)-b-pSar(10, 25, or 50)). In some embodiments, an anionic polymer is a block copolymer of poly-α-L-glutamic acid (PGA)-block-2-(2-(2-aminoethoxy)ethoxy)acetic acid (AEEA). In some embodiments, a PGA-b-AEEA block copolymer has 10, 20, 50, or 100 units of PGA and 4, 8, or 14 units of AEEA. In some embodiments, a PGA-b-AEEA block copolymer is PGA(50)-Ac-AEEA(14), where “Ac” refers to an acetate group.

In some embodiments, an anionic polymer described herein comprises a targeting moiety or imaging moiety. For example, an anionic polymer can include an imaging moiety such as a radionuclide (e.g., ¹⁸F, ¹³¹I, and the like), a chelating moiety (e.g., metal chelating moieties such as DOTA), and/or a fluorophore (e.g., fluorescein).

In some embodiments, a targeting moiety is a derivative of an agent having a strong binding affinity to certain cellular markers on a target cell, thereby enhancing targeting and internalization of the complex and/or the cargo into the target cell. In some embodiments, a targeting moiety is or comprises one or more of a small molecule, a peptide, an aptamer or other nucleic acid sequence, or a nanobody. In some embodiments, a small molecule targeting moiety is folic acid, phenylboronic acid, alendronate, telmisartan, glycyrrhetinic acid, adenosine, a vitamin, or a carbohydrate. In some embodiments, a vitamin is vitamin H (biotin) or vitamin B (flavin) mononucleotide. In some embodiments, a carbohydrate is a monosaccharide selected from glucose, mannose, galactose, fucose, sialic acid, mannose-6-phosphate, and lactobionic acid, or an oligosaccharide comprising one or more monosaccharides described herein. In some embodiments, a peptide is cysteine-arginine-glutamic acid-lysine-alanine (CREKA) peptide of SEQ ID No.1, K237 peptide of SEQ ID NO.2, F3 peptide of SEQ ID No.3, A54 peptide of SEQ ID No. 4, apamin of SEQ ID No. 5, hexapeptide ligand AE, epidermal growth factor, octreotide, RGD (Arg-Gly-Asp) motif with SEQ ID No. 6, iRGD, RGDF peptides, cRGD, U11 peptide, HIV trans-activating transcriptional activator (TAT) peptide, Lyp-1 peptide, Rabies virus glycoprotein (RVG), chlorotoxin (CITx), Aβ-binding peptides (KLVFF) with SEQ ID No. 7, TGNYKALHPHNG (TGN) with SEQ ID No.8, QSHYRHISPAQV(QSH) with SEQ. ID No. 9, Tet-1 peptide, T7 peptide, apolipoprotein, E Peptide(ApoE), Peptide motif B6, angiopep-2, cell-penetrating peptide (CPP), ferritin.

In some embodiments, a peptide is SRLEEELRRRLTE with SEQ. ID No. 10, and is referred to as an “Alfa” peptide. In some embodiment, a composition comprising the Alfa peptide is referred to as being “Alfa-tagged”. In some embodiments, an aptamer is nucleotide-targeted ADN aptamer AS1411, anti-epidermal growth factor receptor aptamer, mucins-1 aptamer, prostate-specific membrane antigen (PSMA) aptamer A9g or PSMA A10. In some embodiments, the nanobody is anti-HER2 nanobody 2Rb17c or EGa1 nanobodies. In some embodiments, a nanobody targets the Alfa peptide (NbALFA). In some embodiments, the nanobody is NbALFA. In some embodiments, the targeting molecule is a multivalent dendron moiety comprising several identical or different targeting motifs.

In some embodiments, an anionic polymer described herein further comprises a stealth linker between the anionic polymer and a targeting moiety. A stealth moiety, as used herein, is any suitable polymer, including, for example, repeating units of ethylene glycol, sarcosine, or AEEA. For example, in some embodiments, an anionic polymer that comprises a targeting moiety is a hetero block co-polymer that is poly(glutamic acid)-block-poly-2-(2-(2-aminoethoxy)ethoxy)acetic acid-Alfa (referred to as PGA-b-Ac-AEEA-Alfa). In some embodiments, an anionic polymer that comprises a targeting moiety is PGA(50)-b-Ac-AEEA(14)-Alfa. In some embodiments, an anionic polymer that comprises a targeting moiety is a block copolymer of poly-α-L-glutamic acid and polysarcosine, and the targeting moiety is an Alfa peptide. In some embodiments, an anionic polymer that comprises a targeting moiety is PGA(50)-b-pSar(50)-Alfa.

In some embodiments, an anionic polymer comprising a targeting moiety is a homopolymer of poly-α-L-glutamic acid and the targeting moiety is an Alfa peptide.

In some embodiments, provided compounds are provided and/or utilized in a salt form (e.g., a pharmaceutically acceptable salt form). Reference to a compound provided herein is understood to include reference to salts thereof, unless otherwise indicated.

### Complex Characteristics

As described herein, a complex comprises a core complex that is encapsulated within an anionic polymer. In some embodiments, a complex that is encapsulated is characterized by a charge ratio of anionic groups in an anionic polymer to cationic groups in a cationic polymer of the core complex. In some embodiments, a charge ratio of an anionic polymer to a cationic polymer is from about 0.5:1 to bout 3:1. In some embodiments, a charge ratio of an anionic polymer to a cationic polymer is form about 0.5:1 to about 2:1. In some embodiments, a charge ratio of an anionic polymer to a cationic polymer is form about 1:1 to about 2:1. In some embodiments, a charge ratio of an anionic polymer to a cationic polymer is form about 1.25:1 to about 2:1. In some embodiments, a charge ratio of an anionic polymer to a cationic polymer is about 1.5:1.

In some embodiments, a complex (e.g., an encapsulated complex) described herein is characterized by a diameter of from about 10 nm to about 200 nm. In some embodiments, a complex (e.g., an encapsulated complex) described herein is characterized by a diameter of from about 10 nm to about 150 nm. In some embodiments, a complex (i.e., an encapsulated complex) described herein is characterized by a diameter of from about 10 nm to about 100 nm. In some embodiments, a complex (e.g., an encapsulated complex) described herein is characterized by a diameter of from about 10 nm to about 70 nm. In some embodiments, a complex described herein is characterized by a diameter of from about 20 nm to about 50 nm. In some embodiments, a complex described herein is characterized by a diameter of about 20 nm, about 25 nm, about 30 nm, about 35 nm, about 40 nm, about 45 nm, or about 50 nm. In some embodiments, a complex described herein is characterized by a diameter of less than about 50 nm. Methods of characterizing a diameter for a complex are described in PCT App. Pub. No. WO 2021/001417.

In some embodiments a composition comprising complexes described herein exhibit uniform particle distribution (i.e., have a polydispersity index (PDI) that is indicative of a collection of particles that are of similar size). For example, in some embodiments, a composition of complexes described herein have a PDI less than 0.5 (e.g., less than 0.5, less than 0.4, less than 0.3).

One advantage of provided complexes, among others, is that a surface charge can be tuned to a desirable value depending on the selection of the anionic polymer. For example, in some embodiments, a surface charge of a provided complex can be from about +25 mV to about −25 mV. Without wishing to be bound by theory, it is hypothesized that increasing an amount of anionic polymer in the complex allows for up to about −25 mV surface charge.

Still further, presently described complexes exhibit improved colloidal stability, for example, across multiple freeze-thaw cycles (e.g., over 1, 2, 3, or more freeze-thaw cycles). Such complexes exhibit said stability over freeze-thaw cycles at both −20° C. and −80° C. In some embodiments, stability of particular complexes is improved using anionic polymers having less than 100 repeating units. In some embodiments, an anionic polymer described herein has less than or equal to 50 total monomeric units. In some embodiments, an anionic polymer described herein has less than or equal to 25 total monomeric units. In some embodiments, an anionic polymer described herein has less than or equal to 10 total monomeric units.

In some embodiments a complex described herein comprises a core complex that is encapsulated by an anionic polymer shell. Determination that a complex comprises a core/shell structure can be by any method known to those of skill in the art. For example, in some embodiments, determination of a core/shell structure can be by microscopic methods, such as Transmission Electron Microscopy (TEM). In TEM, samples are scanned with an electron beam, and electrons are transmitted through the samples then collected, and the signal is then transformed into an image. The distinction of the core from the shell can happen through the application of a staining agent to change the contrast between the components.

In some embodiments, determination of a core/shell structure can be by small-angle scattering methods, such as small-angle x-ray scattering (SAXS). In SAXS, samples are treated with monochromatic X-ray that is then scattered by the electrons of the atoms present in the formulations. SAXS measures the excess electron density, i.e. the density difference between particles and their surrounding medium. Satisfactory distinction between core and shell are obtained provided that the core and the shell differ in scattering intensity of electrons. SAXS can help with giving quantitative info about the sizes and shapes of the particles (that might change upon coating) as well as specific surface modification (like the coating layer thickness).

In some embodiments, determination of a core/shell structure can be by differential scanning calorimetry (DSC). DSC uses a change in temperature to measure a heat quantity, which is radiated or absorbed by the sample. The change in heat capacity for each segment (core and shell) can be used to confirm the existence of the two components (two separate signals are present if components are completely immiscible) and determine quantitatively the weight fractions.

In some embodiments, determination of a core/shell structure can be by infrared spectroscopy (IR), including, for example Fourier transform infrared (FTIR). FTIR is based on the principle that infrared radiation passes through a sample and some of the radiation is absorbed (and recorded). This radiation is converted into energy based on the specific interaction of functional groups with the radiation, generating a spectrum that is a molecular fingerprint of the sample. If new functional groups are introduced in the sample (as consequence of the coating material) the FTIR spectrum will consequently change.

### Methods of Delivery

The present disclosure provides, among other things, a complex (e.g., a pharmaceutical composition or a pharmaceutical formulation, as referred to herein) to be administered to a subject. For example, in some embodiments, a complex is administered as a monotherapy. In some embodiments, a complex is administered as part of a combination therapy. In some embodiments, a concentration of RNA in a pharmaceutical composition described herein is of about 0.01 mg/mL to about 0.5 mg/mL, or about 0.05 mg/mL to about 0.1 mg/mL.

Pharmaceutical compositions may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, MD, 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, an excipient is approved for use in humans and for veterinary use. In some embodiments, an excipient is approved by the United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator.

In some embodiments, pharmaceutical formulations described herein can further comprise a buffering agent. In some embodiments, a buffering agent is selected from 4-(2-hydroxyethyl)-1-piperazineethanesulfonic acid (HEPES), 3-morpholino-2-hydroxypropanesulfonic acid (MOPSO), 2-(N-morpholino)ethanesulfonic acid (MES), Bis-tris buffering systems, carboxylic acid buffering systems, phosphatic acid buffering systems, or citric acid buffering systems.

General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety).

In some embodiments, pharmaceutical compositions provided herein may be formulated with one or more pharmaceutically acceptable carriers or diluents as well as any other known adjuvants and excipients in accordance with conventional techniques such as those disclosed in Remington: The Science and Practice of Pharmacy 21st ed., Lippincott Williams & Wilkins, 2005 (incorporated herein by reference in its entirety).

Pharmaceutical complexes and compositions described herein can be administered by appropriate methods known in the art. As will be appreciated by a skilled artisan, the route and/or mode of administration may depend on a number of factors, including, e.g., but not limited to stability and/or pharmacokinetics and/or pharmacodynamics of pharmaceutical compositions described herein.

In some embodiments, pharmaceutical compositions described herein are formulated for parenteral administration, which includes modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In some embodiments, pharmaceutical compositions described herein are formulated for intravenous administration. In some embodiments, pharmaceutically acceptable carriers that may be useful for intravenous administration include sterile aqueous solutions or dispersions and sterile powders for preparation of sterile injectable solutions or dispersions.

In some particular embodiments, pharmaceutical compositions described herein are formulated for subcutaneous (s.c.) administration. In some particular embodiments, pharmaceutical compositions described herein are formulated for intramuscular (i.m.) administration. In some embodiments, pharmaceutical compositions described herein are formulation for intranasal administration. In some embodiments, pharmaceutical compositions described herein are formulation for intravenous (i.v.) administration.

Therapeutic compositions typically must be sterile and stable under the conditions of manufacture and storage. The composition can be formulated as a solution, dispersion, powder (e.g., lyophilized powder), microemulsion, lipid nanoparticles, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition.

In some embodiments, prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration.

In some embodiments, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Examples of suitable aqueous and nonaqueous carriers which may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the presence of microorganisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into pharmaceutical compositions described herein. In addition, prolonged absorption of the injectable pharmaceutical form may be brought about by the inclusion of agents which delay absorption such as aluminum monostearate and gelatin.

Formulations of pharmaceutical compositions described herein may be prepared by any method known or hereafter developed in the art of pharmacology. In general, such preparatory methods include the step of bringing active ingredient(s) into association with a diluent or another excipient and/or one or more other accessory ingredients, and then, if necessary and/or desirable, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of at least one RNA product produced using a system and/or method described herein.

In some embodiments, an active agent that may be included in a pharmaceutical composition described herein is or comprises a therapeutic agent administered in a combination therapy described herein. Pharmaceutical compositions described herein can be administered in combination therapy, i.e., combined with other agents. In some embodiments, such therapeutic agents may include agents leading to depletion or functional inactivation of regulatory T cells. For example, in some embodiments, a combination therapy can include a provided pharmaceutical composition with at least one immune checkpoint inhibitor.

In some embodiments, pharmaceutical composition described herein may be administered in conjunction with radiotherapy and/or autologous peripheral stem cell or bone marrow transplantation.

In some embodiments, a pharmaceutical composition described herein can be frozen to allow long-term storage.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation.

It is also appreciated that different methods of administration can be used to treat diseases, disorders, or conditions described herein. For example, in some embodiments, a composition comprising a complex described herein is administered a method described herein in one or more doses (e.g., one or more doses by parenteral means, intranasal means, oral, and the like). In some embodiments, a first dose is administered by a first means, and a second dose is administered by the same or different means. For example, in some embodiments, a first dose of a composition comprising a complex described herein is administered by intramuscular, subcutaneous, or intravenous means, and a second dose of a composition comprising a complex described herein is administered by intranasal means. In some embodiments, doses can be administered in a routine manner by any means described herein after an initial dose. For example, in some embodiments, a first dose is administered by intramuscular, subcutaneous, or intravenous means, and subsequent doses are administered by intranasal means.

In some embodiments, a first dose is administered, and a second dose is administered at a period of time after the first dose. In some embodiments, a period of time between a first and second dose is between one day and one year. In some embodiments, a period of time between a first dose and a second dose is between one week and six months. In some embodiments, a period of time between a first dose and a second dose is between one month and three months.

In some embodiments, subsequent doses after a first dose are administered in regular intervals. For example, in some embodiments, a subsequent dose is administered once per year after administration of a first dose.

### Methods of Use

Complexes described herein are useful in the treatment and prophylaxis in a subject of diseases, disorders, and conditions described herein. In some embodiments, a disease, disorder, or condition is an infectious disease, cancer, an autoimmune disease, or a rare disease.

In some embodiments, an infectious disease is caused by or associated with a viral pathogen. In some embodiments, a viral pathogen is of a family selected from poxviridae, rhabdoviridae, filoviridae, paramyxoviridae, hepadnaviridae, coronaviridae, caliciviridae, picornaviridae, reoviridae, retroviridae, and orthomyxoviridae. In some embodiments, an infectious disease is a virus selected from SARS-CoV-2, influenza, Crimean-Congo Hemorhhagic Fever (CCHF), Ebola virus, Lassa virus, Marburg virus, HIV, Nipah virus, and MERS-CoV.

In some embodiments, an infectious disease is caused by or associated with a bacterial pathogen. In some embodiments, a bacterial pathogen is of a species selected from Actinomyces israelii, bacillus antracis, Bacteroides fragilis, Bordetella pertussis, Borrelia burgdorferi, Borrelia garinii, Borrelia afzelii, Borrelia recurrentis, Brucella abortus, Brucella canis, Brucella melitensis, Brucella suis, Campolobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium idphteriae, Ehrlichia canis, Ehrlichia chaffeensis, Enterococcus faecalis, Enterococcus faecium, Escherichia coli, Francisella tularensis, Haemophilus influenzae, Helicobacter pylori, Klebsiella pneumoniae, Legionella pneumophila, Leptospira, Listeria monocytogenes, Mycobacterium leprae, Mycobacterium tuberculosis, Mycoplasma pneumoniae, Neisseria gonorrhoeae, Neisseria meningitidis, Pseudomonas aeruginosa, Nocardia asteroids, Rickettsia ricektssii, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Vibrio cholerae, and Yersinia pestis.

In some embodiments, an infectious disease is caused by or associated with a parasite. In some embodiments, a parasite is of a family selected from Plasmodium, Leishmania, Cryptosporidium, Entamoeba, Trypanosomas, Schistosomes, Ascaris, Echinococcus and Taeniidae.

In some embodiments, a disease, disorder, or condition is a cancer. In some embodiments, a cancer is selected from bladder cancer, breast cancer, colorectal cancer, kidney cancer, lung cancer, lymphoma, melanoma, oral/oropharyngeal cancer, pancreatic cancer, prostate cancer, thyroid cancer, and uterine cancer.

In some embodiments, a disease, disorder, or condition is a genetic disorder. In some embodiments, a genetic disorder is associated with a gain-of-function mutation or a loss-of-function mutation.

In some embodiments, a disease, disorder, or condition is an autoimmune disease. In some embodiments, an autoimmune disease is selected from addison disease, celiac disease, rheumatoid arthritis, lupus, inflammatory bowel disease, dermatomyositis, multiple sclerosis, diabetes, guillain-barre syndrome, chronic inflammatory demyelinating polyneuropathy, psoriasis, pernicious anemia, graves' disease, hashimoto's thyroiditis, myasthenia gravis, and vasculitis sjörgen syndrome.

In some embodiments, a disease, disorder, or condition is a rare disease. As described herein, a rare disease refers to a life-threatening or chronically debilitating diseases which are of such low prevalence (e.g., fewer than 1/2000 people) that special combined efforts are needed to address them.

In some embodiments, the present disclosure provides complexes that can selectively target particular systems within a body. As used herein, reference to “targeting” a particular system refers to causing increased expression of RNA derived from cargo in the complex in the desired system. For example, in some embodiments, complexes described herein can selectively target the lungs, liver, spleen, heart, brain, lymph nodes, bladder, kidneys, and pancreas. As described herein, a complex “selectively targets” an organ when a single target expresses mRNA in an amount that is 65% or greater than expression in other organs post administration (e.g., 65% or more of mRNA throughout the body is expressed from a single organ, with the remaining 35% distributed between one or more different organs). In some embodiments, a complex described herein selectively targets the lungs. In some embodiments, a complex described herein selectively targets the liver. In some embodiments, a complex described herein selectively targets the spleen. In some embodiments, a complex described herein selectively targets the heart.

### EXEMPLARY EMBODIMENTS

Embodiment 1. A complex comprising a cationic polymer, an anionic polymer, and a monomeric RNA molecule, wherein the cationic polymer and the monomeric RNA molecule form a core complex encapsulated by the anionic polymer.

Embodiment 2. The complex of Embodiment 1, wherein the cationic polymer is or comprises copolymers of one or more of poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

Embodiment 3. The complex of Embodiment 1 or 2, wherein the cationic polymer is a homopolymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

Embodiment 4. The complex of Embodiments 2 or 3, wherein the cationic polymer is linear poly(ethylenimine).

Embodiment 5. The complex of Embodiment 4, wherein the linear poly(ethylenimine) has a Mₙ of about 600 Da to about 400,000 Da.

Embodiment 6. The complex of Embodiment 5, wherein the linear poly(ethylenimine) has a Mₙ of about 10,000 Da to about 120,000 Da.

Embodiment 7. The complex of any one of Embodiments 1 or 2, wherein the cationic polymer is a linear block-co-polymer comprising poly(ethylenimine) and poly(propylenimine).

Embodiment 8. The complex of Embodiment 1, wherein the cationic polymer is a polymer or copolymer comprising blocks of formula I and/or II:

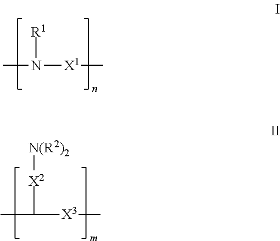

or a pharmaceutically acceptable salt thereof, wherein:
R¹ is H, —C(O)-optionally substituted C₁-C₆ aliphatic, optionally substituted C₁-C₆ aliphatic or G;
each R² is independently H, optionally substituted C₁-C₆ aliphatic, or G;
X¹ and X³ are each independently C1-C₆ aliphatic;
X² is a bond or optionally substituted C₁-C₆ aliphatic;
m is an integer between 2 and 2000;
n is an integer between 2 and 2000;
each G is independently:

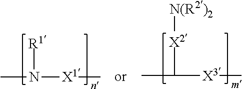

R^{1′} is H, —C(O)-optionally substituted C₁-C₆ aliphatic, optionally substituted C₁-C₆ aliphatic, or G′;
each R^{2′} is independently H, optionally substituted C₁-C₆ aliphatic, or G′;
X¹ and X^{3′} are each independently optionally substituted C₁-C₆ aliphatic;
X^{2′} is a bond or optionally substituted C₁-C₆ aliphatic;
n′ is an integer between 2 and 2000;
m′ is an integer between 2 and 2000;
each G′ is independently:

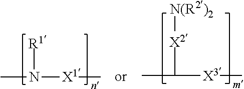

R^{1″} is H, —C(O)-optionally substituted C₁-C₆ aliphatic, or optionally substituted C₁-C₆ aliphatic;
each R^{2″} is independently H or C1-C₆ aliphatic;
X^{1″} and X^{3″} are each independently optionally substituted C₁-C₆ aliphatic;
X^{2″} is a bond or optionally substituted C₁-C₆ aliphatic;
n″ is an integer between 2 and 2000; and
m″ is an integer between 2 and 2000.

Embodiment 9. The complex of any one of Embodiments 1-8, wherein the anionic polymer is a homopolymer.

Embodiment 10. The complex of any one of Embodiments 1-9, wherein the anionic polymer is a homopolymer selected from poly-L-glutamic acid, poly-L-aspartic acid, a polysaccharide, poly(2-methoxycarbonylethyl-2-oxazoline), poly(2-methoxycarbonylpropyl-2-oxazoline), poly(2-methoxycarbonylethyl-2-oxazine), poly(2-methoxycarbonylpropyl-2-oxazine), and polyphosphate.

Embodiment 11. The complex of any one of Embodiments 9 or 10, wherein the anionic polymer comprises from about 10 to about 100 repeating monomeric units.

Embodiment 12. The complex of any one of Embodiments 1-8, wherein the anionic polymer is a heteropolymer.

Embodiment 13. The complex of Embodiment 12, wherein the heteropolymer comprises monomers of glutamic acid, aspartic acid, ethylene glycol, propylene glycol, sarcosine, phosphates, 2-methyl-2-oxazoline, 2-methoxycarbonylethyl-2-oxazoline, 2-ethyl-2-oxazoline, 2-methyl-2-oxazine, 2-methoxycarbonylethyl-2-oxazine, 2-methoxycarbonylpropyl-2-oxazoline, 2-methoxycarbonylpropyl-2-oxazine, 2-(2-(2-aminoethoxy)ethoxy)acetic acid, and/or 2-(2-(2-methylaminoethoxy)ethoxy)acetic acid.

Embodiment 14. The complex of Embodiments 12 or 13, wherein the anionic polymer comprises about 10 to about 100 monomeric units.

Embodiment 15. The complex of any one of Embodiments 1-8, wherein the anionic polymer is a block-co-polymer.

Embodiment 16. The complex of Embodiment 15, wherein the block-co-polymer comprises blocks of polymers selected from the group consisting of poly(2-methoxycarbonylethyl-2-oxazoline), poly(2-methyl-2-oxazoline), poly-L-glutamic acid, poly-L-aspartic acid, polyphosphates, poly(N-methyl-sarcosine), poly(ethylene glycol), poly(propylene glycol), poly(2-methoxycarbonylpropyl-2-oxazoline), poly(2-methoxycarbonylethyl-2-oxazine), poly(2-methoxycarbonylpropyl-2-oxazine), poly-2-(2-(2-aminoethoxy)ethoxy)acetic acid, and poly-2-(2-(2-methylaminoethoxy)ethoxy)acetic acid.

Embodiment 17. The complex of Embodiment 16, wherein the block-co-polymer comprises an anionic block and a stealth block.

Embodiment 18. The complex of Embodiment 17, wherein the anionic block is selected from poly(2-methoxycarbonylethyl-2-oxazoline), poly-L-glutamic acid, poly-L-aspartic acid, polyphosphate, poly(2-methoxycarbonylpropyl-2-oxazoline), poly(2-methoxycarbonylethyl-2-oxazine), and poly(2-methoxycarbonylpropyl-2-oxazoline).

Embodiment 19. The complex of Embodiments 17 or 18, wherein the stealth block is selected from the group consisting of poly(ethylene glycol), poly(propylene glycol), poly(2-methyl-2-oxazoline), poly(2-ethyl-2-oxazoline), poly(2-methyl-2-oxazine), poly(N-methyl-sarcosine), poly-2-(2-(2-aminoethoxy)ethoxy)acetic acid, and poly-2-(2-(2-methylaminoethoxy)ethoxy)acetic acid.

Embodiment 20. The complex of any one of Embodiments 15-19, wherein each block comprises about 10 to about 100 repeating monomeric units.

Embodiment 21. The complex of Embodiment 1, wherein the cationic polymer is a linear poly(ethyleneimine), and the anionic polymer is a block co-polymer comprising block selected from poly-L-glutamic acid, poly-L-aspartic acid, and poly-2-(2-(2-aminoethoxy)ethoxy)acetic acid.

Embodiment 22. The complex of any one of Embodiments 1-21, wherein the anionic polymer comprises a targeting moiety.

Embodiment 23. The complex of Embodiment 22, wherein the targeting moiety is an Alfa peptide.

Embodiment 24. The complex of any one of Embodiments 1-23, wherein a charge ratio of anionic group in the anionic polymer to cationic groups in the cationic polymer is from about 0.25:1 to about 3:1.

Embodiment 25. The complex of Embodiment 24, wherein the charge ratio of anionic group in the anionic polymer to cationic groups in the cationic polymer is about 1.5:1.

Embodiment 26. The complex of any one of Embodiments 1-25, further comprising a buffering agent.

Embodiment 27. The complex of Embodiment 26, wherein the buffering agent is selected from the group consisting of 4-(2-hydroxyethyl)-1-piperazineethanesulfonic acid (HEPES), 3-morpholino-2-hydroxypropanesulfonic acid (MOPSO), 2-(N-morpholino)ethanesulfonic acid (MES), 2-Amino-2(hydroxymethyl)propane-1,3-diol (TRIS), Bis-tris buffering systems, carboxylic acid buffering systems, phosphatic acid buffering systems, and citric acid buffering systems.

Embodiment 28. The complex of any one of Embodiments 1, 9-20, or 22-27, wherein the cationic polymer is or comprises a polyamine derivative.

Embodiment 29. The complex of Embodiment 28, wherein the polyamine derivative is:

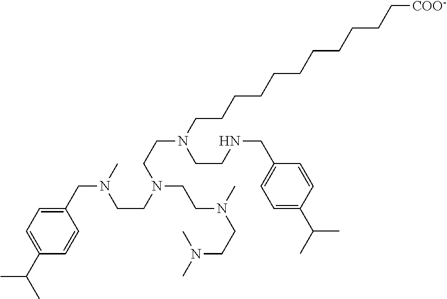

Embodiment 30. The complex of any one of Embodiments 1-29, wherein the RNA is mRNA, modRNA, saRNA, or taRNA.

Embodiment 31. The complex of any one of Embodiments 1-30, wherein the complex has a diameter of about 10 nm to about 150 nm.

Embodiment 32. The complex of any one of Embodiments 1-31, wherein the complex has a diameter of about 20 nm to about 50 nm.

Embodiment 33. A method of increasing or causing increased expression of RNA in a target in a subject comprising administering to the subject the complex of any one of Embodiments 1-32.

Embodiment 34. The method of Embodiment 33, wherein the target is selected from the lungs, liver, spleen, heart, brain, lymph nodes, bladder, kidneys, and pancreas.

Embodiment 35. A method of treating a disease, disorder, or condition in a subject comprising administering to the subject a complex of any one of Embodiments 1-32.

Embodiment 36. The method of Embodiment 35, wherein the disease, disorder, or condition is an infectious disease, cancer, a genetic disorder, an autoimmune disease, or a rare disease.

Embodiment 37. The method of any one of Embodiments 33-36, wherein the complex is administered parenterally.

Embodiment 38. The method of Embodiment 37, wherein the complex is administered intramuscularly, subcutaneously, or intravenously.

Embodiment 39. The method of any one of Embodiments 33-36, wherein the complex is administered intranasally.

Embodiment 40. The method of any one of Embodiments 33-36, wherein a first dose of the complex is administered by a first route, and a second dose of the complex is administered by a second route.

Embodiment 41. The method of Embodiment 40, wherein the first route is parenteral.

Embodiment 42. The method of Embodiments 40 or 41, wherein the second route is intranasal.

Embodiment 43. A complex of any one of Embodiments 1-32, for use as a medicament.

Embodiment 44. A complex of any one of Embodiments 1-32, for use in the treatment and/or prevention of a disease, disorder, or condition, wherein the disease, disorder, or condition is an infectious disease, cancer, a genetic disorder, an autoimmune disease, or a rare disease.

### EXAMPLES

As described in the Examples below, in certain exemplary embodiments, compounds are prepared according to the following general procedures. It will be appreciated that, although the general methods depict the synthesis of certain compounds of the present disclosure, the following general methods and other methods known to one of ordinary skill in the art can be applied to all compounds and subclasses and species of each of these compounds, as described herein.

Table of Abbreviations

| | |
|---|---|
| -b- | -block- |
| Da | Dalton |
| DP | Degree of polymerization |
| i.m. | Intramuscular |
| i.v. | Intravenous |
| MBG | MES buffered glucose |
| MES | 2-(N-morpholino)ethanesulfonic acid |
| pC2MestOx | Poly(2-methoxycarbonylethyl-2-oxazoline) |
| PEI | Polyethylenimine |
| PGA | poly-α-L-glutamic acid |
| pMeOx | poly(2-methyl-2-oxazoline) |
| pSar | Poly(N-methyl-sarcosine) |
| AEEA | 2-(2-(2-aminoethoxy)ethoxy)acetic acid |
| pAEEA | poly(2-(2-(2-aminoethoxy)ethoxy)acetic acid) |

### Example 1

A composition comprising saRNA was complexed at N/P12 with linear polyethylenimine (PEI; 500DP or 22.5 kDa), in MBG Buffer (final concentration 5% w/v Glucose, 10 mM MES, pH 6.1) and at RNA final concentration of 0.1 mg/mL. The mixing ratio of the RNA containing solution and the polymer containing solution was 99:1 (v:v). The formation of ternary formulations of saRNA were produced after addition of either a homopolymer of poly-α-L-glutamic acid (PGA50) or a block-copolymer of poly-α-L-glutamic acid-block-poly-N-methyl-sarcosine (PGA(50)-b-pSar(50) with 50 repeating units of each block) or a homopolymer of poly(2-methoxycarbonylethyl-2-oxazoline) (pC2MestOx(50) a polymer having 50 repeating units) or a block-copolymer of poly(2-methoxycarbonylethyl-2-oxazoline)-block-poly(2-methyl-2-oxazoline) (pC2MestOx(50)-b-pMeOx(50) with 50 repeating units each block) and having a charge ratio of anion:PEI of 1.5. The charge ratio anion:PEI is based on ratio of the negative charge in anionic polymer to the positive charges present in PEI.

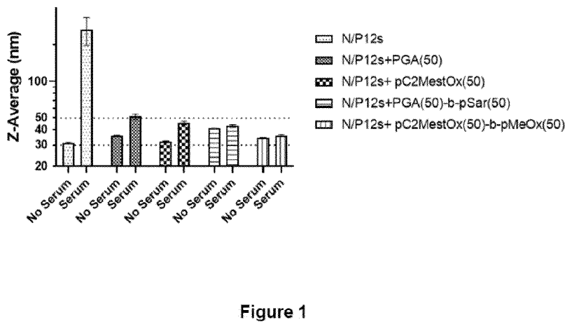

After addition of the appropriate amount of each anionic polymer, the concentration was adjusted to 0.08 mg/mL RNA in all samples in MBG Buffer (final concentration 5% w/v Glucose, 10 mM MES, pH 6.1). A control group was also included where the sample was further diluted with buffer to match the concentration of 0.08 mg/mL RNA seen in the other samples. The composition comprising saRNA and buffer or just buffer was used as a control is referenced simply as “N/P12s” in FIG. 1. The samples colloidal stability after serum incubation was investigated by addition of either human serum to the samples (to provide a final concentration of 10% v/v concentration of human serum) or buffer in order to have a final RNA concentration of 0.05 mg/mL. All samples were diluted with human serum or buffer, incubated for 30 minutes at 37° C. After 30 minutes, the samples are cooled in an ice bath at 4° C. for 10 minutes and size of the complexes is measured with dynamic light scattering. FIG. 1 is a bar graph illustrating the average hydrodynamic diameter of formulations comprising saRNA after incubation in 10% v/v human serum at 37° C. for 30 minutes.

### Example 2

A composition comprising saRNA is complexed at N/P12 with linear PEI (500DP or 22.5 kDa), in MBG Buffer (final concentration 5% w/v Glucose, 10 mM MES, pH 6.1) and at RNA final concentration of 0.1 mg/ml, where the mixing ratio of the RNA containing solution and the polymer containing solution is 99:1 (v:v). The formation of ternary formulations of saRNA are produced after addition of poly-α-L-glutamic acid of different lengths (10, 20, 50 or 100 repeating units, referred to as PGA(10) or PGA(20) or PGA(50) or PGA(100)) or a block-copolymer of poly-α-L-glutamic acid-block (PGA)-poly-N-methyl-sarcosine(pSar) with a variation of the length of pSar (10, 25, or 50 repeating units, hereinafter PGA(50)-b-pSar(10) or PGA(50)-b-pSar(25) or PGA(50)-b-pSar(50)) within a range of concentration that varies depending on the desired final charge ratio of anion to PEI. The charge ratio anion:PEI is based on the negative charges in anionic polymer to the positive charges present in PEI.

After addition of the appropriate amount of each anionic polymer, the concentration of RNA is adjusted to 0.08 mg/mL in all samples in MBG Buffer (final concentration 5% w/v Glucose, 10 mM MES, pH 6.1). In total 8 different charges ratios anion:PEI are tested for each type of PGA polymer. One control sample is tested that includes only RNA and buffer. The samples colloidal stability after serum incubation is investigated by addition of either human serum to the samples (to provide a final concentration of 10% v/v of human serum) or of buffer to provide a final concentration of 0.05 mg/mL. All samples, either diluted with human serum or buffer, are incubated for 30 minutes at 37° C. After the 30 minutes, the samples are cooled in an ice bath at 4° C. for 10 minutes and the size of the complexes is measured with dynamic light scattering and the biological performance is evaluated in-vitro.

### Example 3

A composition comprising saRNA is complexed with PEI and subsequently complexed with one of five different anions to provide ternary formulations for testing in BALB/c mice. Twenty-one BALB/c mice are divided into seven different groups to receive an intramuscular dose of 1 μg of a composition comprising RNA in each leg. One group receives only the control examples of either Example 1 or 2. The compositions comprising saRNA are complexed at N/P12 with PEI, in MBG Buffer (final concentration 5% w/v glucose, 10 mM MES, pH 6.1) and at RNA final concentration of 0.125 mg/mL, where the mixing ratio of the RNA containing solution and the polymer containing solution was 99:1 (v:v).

The five subsequent ternary formulations are prepared by the selective addition of the appropriate amount of either PGA(10) or PGA(20) or PGA(50) or PGA(100) or PGA(50)-b-pSar(50) at a charge ratio anion:PEI of 1.5 in MBG Buffer (final concentration 5% w/v Glucose, 10 mM MES, pH 6.1) and obtained a final RNA concentration of 0.1 mg/mL. The charge ratio anion:PEI is based on the negative charges in the anionic polymer to the positive charges present in PEI. The BALB/c mice received the following formulations:

| Group | Formulation |
|---|---|
| 1 | saRNA and PEI |
| 2 | saRNA, PEI, PGA(10) |
| 3 | saRNA, PEI, PGA(20) |
| 4 | saRNA, PEI, PGA(50) |
| 5 | saRNA, PEI, PGA(100) a |
| 6 | saRNA, PEI, PGA(50)-b-pSar(50) |
| 7 | MGB Buffer (control) |

All the groups received the same amount of formulated luciferase encoding saRNA (1 μg), applied i.m. to each leg. At six time points (6 h, 24 h, 72 h, 6 d, 9 d, 20 d) in-vivo luciferase expression in applied tissue was measured.

### Example 4

A composition comprising saRNA is complexed with PEI and subsequently complexed with one of four different anionic polymers to provide ternary formulations. Thirty BALB/c mice are divided into five groups. The compositions comprising saRNA are complexed at N/P12 with PEI, in MBG Buffer (final concentration 5% w/v glucose, 10 mM MES, pH 6.1) and at RNA final concentration of 0.125 mg/mL, where the mixing ratio of the RNA containing solution and the polymer containing solution was 99:1 (v:v).

The four subsequent ternary formulations (formulations 2-5) are prepared by the selective addition of the appropriate amount of either PGA(50) or PGA(50)-b-pSar(10) or PGA(50)-b-pSar(25) or PGA(50)-b-pSar(50) at a charge ratio anion:PEI of 1.5 in MBG Buffer (final concentration 5% w/v Glucose, 10 mM MES, pH 6.1) to provide a final RNA concentration of 0.1 mg/mL. The charge ratio anion:PEI is based on the negative charges in the anionic polymer to the positive charges present in PEI.

The five mice groups are given the following formulations:

| Group | Formulation |
|---|---|
| 1 | saRNA, PEI |
| 2 | saRNA, PEI, PGA(50) |
| 3 | saRNA, PEI, PGA(50)-b-pSar(10) |
| 4 | saRNA, PEI, PGA(50)-b-pSar(25) |
| 5 | saRNA, PEI, PGA(50)-b-pSar(50) |

All the groups receive the same amount of formulated luciferase encoding saRNA (1 μg), applied i.v. to the tail vein. After 24 hours of injection, in-vivo and ex-vivo luciferase expression is measured.

### Example 5

### Preparation of Binary Complexes

A binary formulation consisting of Luciferase saRNA was complexed at N/P12 with linear PEI 500 (H[C₂H₄NH·HCl]500OH with Mw 54.960 kDa). Manufacturing was carried out using a syringe pump-based fluid path manufacturing process with a Y-mixing element at total flow ratio of 250 mL/min and 3 to 1 mixing ratio (RNA to Polymer phase) were used. Binary formulation, or core polyplexes (PLXs), were manufactured at RNA final concentration of 0.1 mg/mL in a 1X MBG Buffer (final concentration 5% w/v Glucose, 10 mM MES, pH 6.0). These binary saRNA-PEI PLXs are about 35 nm in diameter and have a positive zeta potential of 20 to 25 mV. No free RNA is detected using Agarose gel electrophoresis (AGE). PLXs are freezable and no size increase was observed at −80° C. for at least 3 freeze thaw cycles. Binary saRNA complexes present a monomeric RNA content of 40% of the total RNA.

A binary formulation consisting of Luciferase modRNA was complexed at N/P12 with linear PEI 500 (H[C₂H₄NH·HCl]500OH) with Mw 54.960 kDa). Manufacturing is carried out using a syringe pump-based fluid path manufacturing process with a T-mixing element. Final buffer composition consists of MBG Buffer (final concentration 5% w/v Glucose, 10 mM MES, pH 6.0) and RNA final concentration of 0.125 mg/ml. These binary modRNA-PEI PLXs are about 30 nm in diameter and a positive zeta potential of 20 to 25 mV. No free RNA is detected using Agarose gel electrophoresis (AGE). PLXs are freezable and no size increase is observed after thawing at −80° C. Binary modRNA complexes present high monomeric RNA content, typically 80% of total RNA is in monomeric form.

### Preparation of Ternary Complexes

The formation of Alfa-tagged ternary formulations of saRNA was produced after addition of the homopolymer of Poly-α-L-glutamic acid (PGA(X), with X=50 or 100 repetitive units) equipped with Alfa-peptide sequence. The formation of ternary complexes is within a range of Glu/N charge ratio from 0 to 3. Final ternary formulations have concentration of 0.08 mg/ml RNA and storage matrix 1X MBG Buffer (5% w/v Glucose, 10 mM MES, pH 6.0).

The ternary formulations of modRNA are produced after addition of a Alfa-tagged block-copolymer of Poly-α-L-glutamic acid-block-poly-N-methyl-sarcosine (PGA(50)-b-pSar(50)-Alfa or a Alfa-tagged block-copolymer of Poly-α-L-glutamic acid-block-poly-N—Ac-AEEA14 (PGA(50)-b-Ac-AEEA(14)-Alfa). The formation of ternary complexes is studied with a range of Glu/N charge ratio from 0 to 3. Final ternary formulations have concentration of 0.1 mg/ml RNA and storage matrix 1X MBG Buffer (5% w/v Glucose, 10 mM MES, pH 6.0).

### Characterization of Binary and Ternary Particles

Physicochemical characterization of binary and ternary saRNA-PLXs and modRNA-PLXs includes Z average size and polydispersity index (PDI) using Wyatt dynamic light scattering (DLS). Surface charge zeta potential was measured using Malvern Zetasizer. Variations of zeta potential, as a consequence of the change in the charge exposed by the particles surface, further confirm the systematic coating of core polyplexes with the anionic polymer. Free RNA content was measured by agarose gel electrophoresis (AGE). Frozen stability studies were carried out by measuring particle size distribution using Wyatt for three consecutive freeze-thaw cycles. In those cycles, particles were frozen at either −20° C. or −80° C. overnight, thawed at room temperature for 1 hour, measured by standard Wyatt protocol and frozen again at the same temperature. The procedure is repeated three times, unless large size increases are observed.

Monomeric RNA was evaluated with a centrifugation and RNA quantification method. Briefly, 400 μl of binary PLX is filled in a 1.5 mL Eppendorf® tube and centrifuged at 20,000 rcf, 4° C. for 1.5 h (accelerating slope 9, slowing 5). A supernatant of 250 μl was taken out of each centrifuged formulation. The RNA concentration of the supernatant (where monomeric RNA is present) is measured and put in relation to the overall RNA content, giving the monomeric RNA of the formulation as a percentage of the total RNA. This method has been proved comparable to an Ultracentrifugation assay where samples are measured with analytic ultracentrifuge and light absorption is measured at two different wavelengths: 255 nm and 650 nm. MBG sample buffer is used as blank sample for the measurements. Samples are centrifuged at 80,000×g at room temperature.

An example of a typical result for modRNA PEI-PLXs is shown in Table 1. In this example, to derive the monomeric RNA content of the formulation, the ratio of the RNA concentration from the centrifuged PLXs over the uncentrifuged one is considered, and this ratio gives a result of 70% monomeric RNA content.

TABLE 1
Results from monomeric RNA assay for modRNA PEI-PLXs

| Sample | RNA concentration (mg/mL) |
|---|---|
| Centrifuged modRNA PEI-PLX | 0.804 |
| Uncentrifuged modRNA PEI-PLX | 0.1165 |

RNA concentration is quantified with a Nanodrop Spectrophotometer. The microvolume option is used for the quantification of RNA, more specifically 1.5 μl of formulated RNA are used. For the calculation of the RNA concentration, 260/280 and 260/230 nm purity ratios are considered, and formulation buffer is used as blanking solution for the RNA quantification of each sample.

Evaluation of Alfa tagged block-copolymer of Poly-α-L-glutamic acid-block-poly-N-methyl-sarcosine, Poly-α-L-glutamic acid and Poly-α-L-glutamic acid-block-poly-N— Ac-AEEA to form ternary complexes with modRNA-PEI polyplexes.

The formation of ternary formulations of modRNA are produced after addition of either Alfa-tagged block-copolymer of Poly-α-L-glutamic acid-block-poly-N-methyl-sarcosine (PGA(50)-b-pSar(50)-Alfa), an Alfa-tagged homopolymer of Poly-α-L-glutamic acid (PGA(X)-Alfa, with X=50, 100 repeating monomeric units) or an Alfa-tagged block-copolymer of Poly-α-L-glutamic acid-block-poly-N—Ac-AEEA (PGA(50)-b-Ac-AEEA(14)-Alfa).

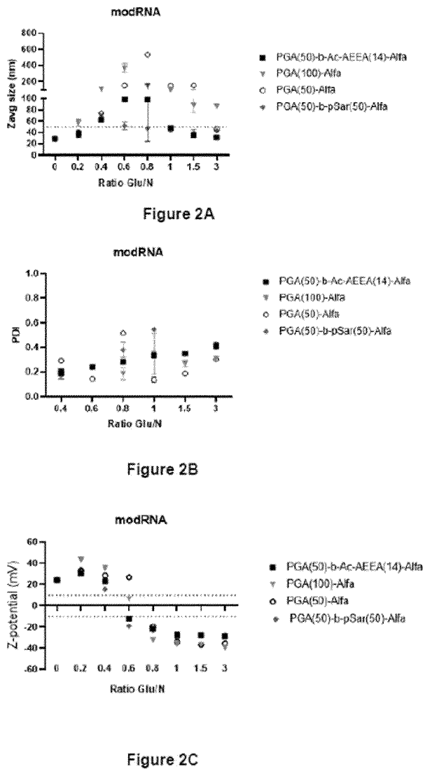

Ternary complexes with PGA(50)-b-pSar(50)-Alfa, PGA(50)-b-Ac-AEEA(14)-Alfa, PGA(50)-Alfa, and PGA(100)-Alfa can be successfully formed with sizes being a function of the polymer used and the Glu/N ratio tested (FIG. 2A). The presence of a stealth moiety as a linker between PGA and Alfa peptide (pSar(50) and pAEEA(14)) allows for particles that have a Z average size below 100 nm.

Across the majority of Glu/N tested, ternary complexes show acceptable distributions, with PDI values≤0.4 and high PDI are usually obtained at high Glu/N ratios (FIG. 2B). At the same time, surface charge of the particles can be tuned, as with increasing amounts of anionic polymer, zeta potential values completely shift from approximately +25 mV to −25 mV as seen in FIG. 2C.

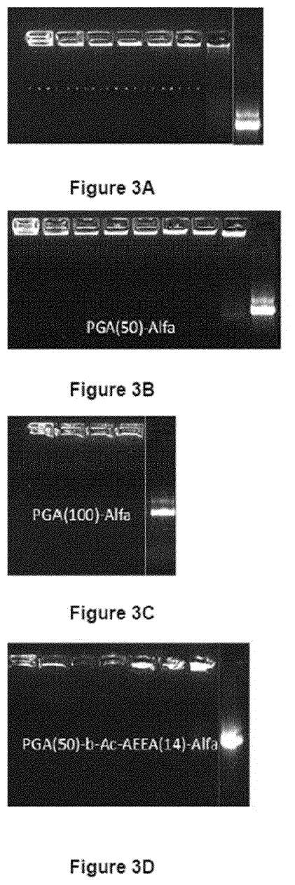

Agarose Gel Electrophoresis (AGE) analysis shows that Alfa-tagged modRNA ternary complexes can be formulated within certain Glu/N ratios achieving fully RNA encapsulation (FIGS. 3A-D).

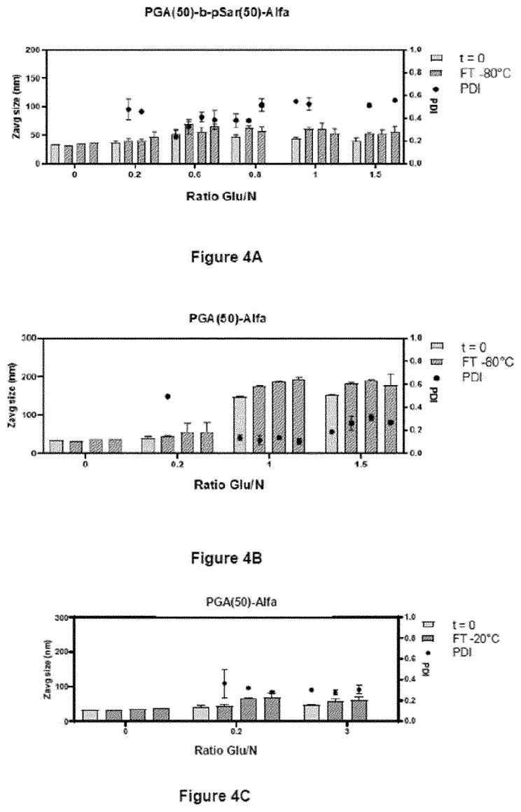

Ternary complexes coated with Alfa-tagged polymers can be successfully stabilized at both −80° C. and −20° C., up to three freeze thaw cycles and in a wide range of Glu/N ratios, thus ensuring frozen stability of particles positively, neutral and negatively charged (FIGS. 4A-F). Without wishing to be bound by theory, it is understood from frozen stability data that a stealth moiety is useful for ensuring stability of polymeric complexes with neutral zeta potential as seen in the case of PGA(50)-b-pSar(50)-Alfa (compare with PGA(50)-Alfa from FIGS. 4A-F).

Overall, results show that modRNA-based complexes can be decorated, through coating with Alfa-tagged polymers, with functionalities that further enable active targeting of these delivery systems and achieve organ tropism.

Evaluation of Homopolymer Alfa-Tagged Poly-α-L-Glutamic Acid Length to Form Ternary Complexes with saRNA-PEI Polyplexes.

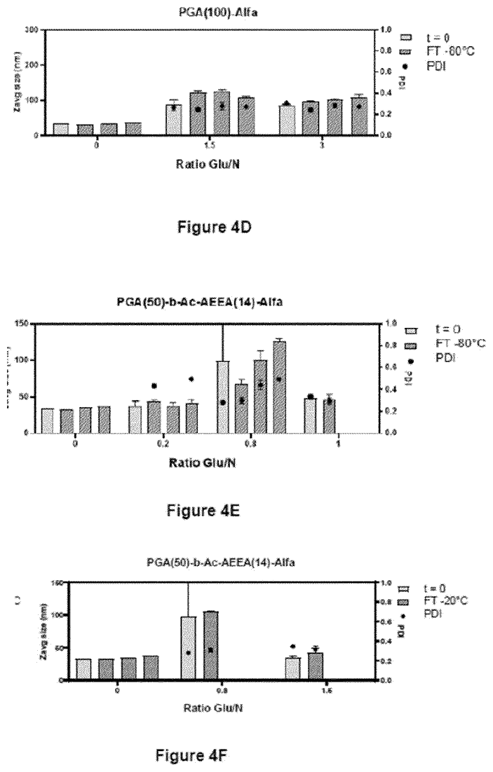

Physicochemical characterization showed that Alfa-tagged ternary complexes can be formed with both PGA(50)-Alfa and PGA(100)-Alfa, in a size range between 40 and 200 nm, with larger particle sizes obtained at Glu/N 0.8-1 (FIGS. 5A-C). Particle size remains relatively small in either very small (0.2) or very large (3) Glu/N ratios. PDI of the particles, as shown in FIGS. 5A-C, is ≤0.4. Without wishing to be bound by theory, it is understood that surface charge of the particles can be tuned with increasing amounts of ternary polymer and can thereby shift from approximately +25 mV to −30 mV as seen in FIGS. 5A-C.

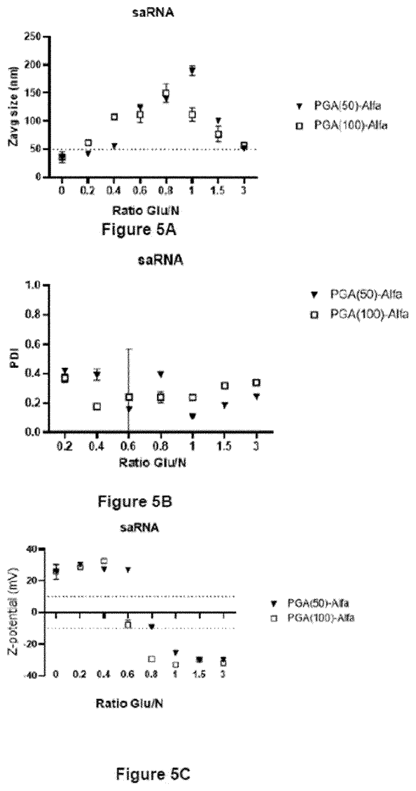

Agarose gel electrophoresis (AGE) analysis shows RNA can be successfully encapsulated up when saRNA binary polyplexes are coated with both PGA(50)-Alfa and PGA(100)-Alfa (FIG. 6).

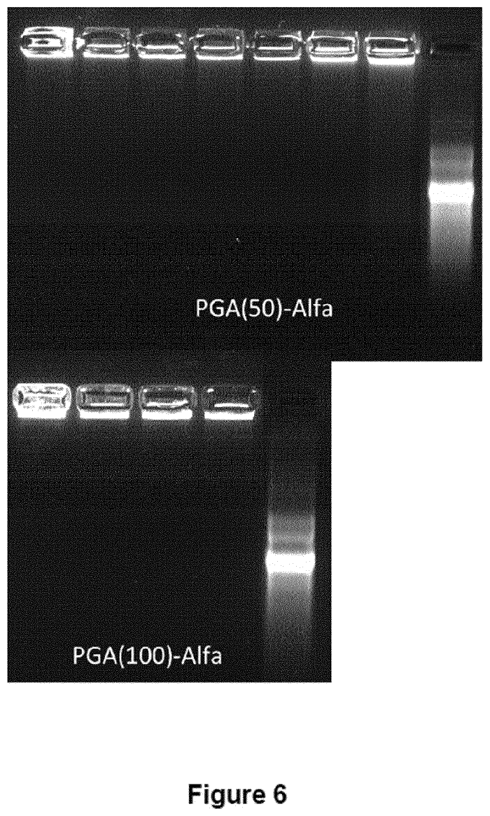

Frozen stability studies show that for PGA(50)-Alfa colloids are stable up to 3 freeze thaw cycles at low amounts of PGA (e.g., a Glu/N ratio of 0.2), when particles are still highly positive charged. For the same polymer length, −80° C. stability can be achieved also at higher Glu/N ratios (0.8). Increasing the polymer length allows to achieve colloidal stability at −20° C. and −80° C. also for negatively charged complexes (Glu/N ratios of 1 and 1.5) (FIGS. 7A-C).

Evaluation of Alfa Tagged Block-Copolymer of Poly-α-L-Glutamic Acid-Block-Poly-N-Methyl-Sarcosine and Poly-α-L-Glutamic Acid-Block-Poly-N—Ac-AEEA14 to Form Ternary Complexes with saRNA-PEI Polyplexes.

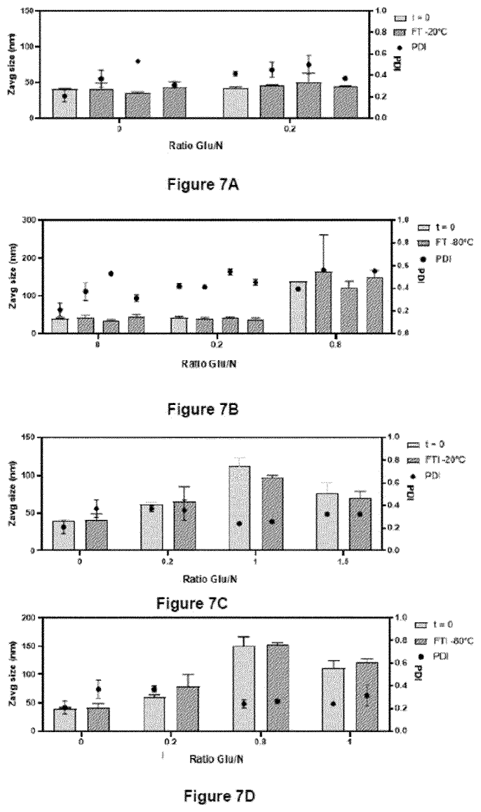

Physicochemical characterization shows that ternary complexes with PGA(50)-b-pSar(50)-Alfa and PGA(50)-b-Ac-AEEA(14)-Alfa can be successfully formed maintaining sizes between 40 and 60 nm (FIG. 8A). In the majority of Glu/N ratios no significant differences are observed in terms of sizes between the two different stealth moieties used (pSar(50) and pAEEA(14)). PDI values≤0.4 are obtained for both anionic polymers (FIG. 8B). Without wishing to be bound by theory, it is hypothesized that when a stealth moiety is present as linker between the anion polymer and the Alfa moiety (pSar(50) and pAEEA(14)), the sizes of the ternary complexes can be maintained below 70 nm.

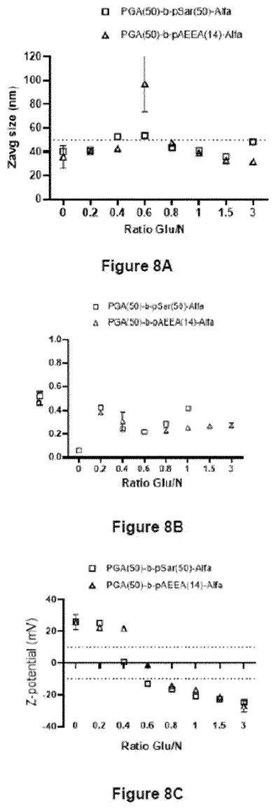

Without wishing to be bound by theory, it is understood that surface charge of the particles can be tuned by increasing amounts of anionic polymer, to allow zeta potential values to completely shift from approximately +25 mV to −25 mV as seen in FIG. 8C. AGE results shows the saRNA can successfully encapsulated upon coating of binary polyplexes with PGA(50)-b-pSar(50)-Alfa and PGA(50)-b-Ac-AEEA(14)-Alfa (FIGS. 9A-B).

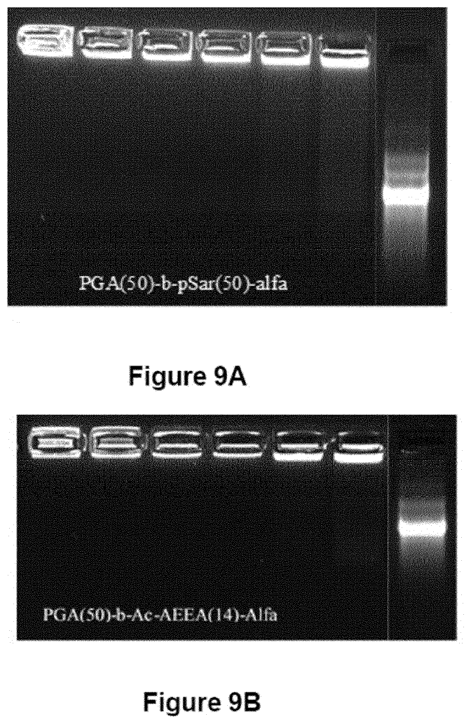

Ternary complexes coated with PGA(50)-b-pSar(50)-Alfa can be successfully stabilized at both −80° C. and −20° C., up to three freeze thaw cycles and in a broad range of Glu/N ratios, thus ensuring frozen stability of particles positively, neutral and negatively charged. This result is particularly important as it broadens the applications of drug delivery systems as a function of specific routes of administration. Ternary complexes obtained with PGA(50)-b-Ac-AEEA(14)-Alfa on the other hand, show good frozen stability at low Glu/N ratios (0.2) (FIGS. 10A-D).

Moreover, a stabilizing effect can be identified in the case of PGA(50)-b-pSar(50)-Alfa polymer: the presence of the alfa-tag on the anion polymer improves stability of negatively charged particles (high Glu/N ratios) (FIGS. 10A and B) compared to anion polymer that does not bear the alfa-tag.

Without wishing to be bound by theory, it is hypothesized that a stealth moiety ensures frozen stability of polymeric complexes with neutral or negative zeta potential and that pSar(50) is particularly efficient in maintaining colloidal properties upon repeated freeze thawing.

### Example 6

### Preparation of Mixed RNA-and-DNA/Viromer Core Particles

A nucleic acid mixture containing Thy1.1 RNA and Nanoplasmid Venus DNA was mixed with the acidified cationic polymer (Viromer VL3 in 0.5% acetic acid) to form the core polyplex particle at a concentration greater than 0.1 g/L at an N/P ratio (ratio of cationic or ionizable nitrogens in the polymer/phosphorus o nucleic acid backbone) of ≥7.5. It was found that conducting this mixing in a microfluidic device improved reproducibility and allowed control over the particle size, compared to mixing by a hand-held pipette—although both methods yield working particles. Subsequently, a buffer concentrate was added to achieve a final concentration of 10 mM HEPES and 10% Trehalose (“1×HBT”) at a pH of approximately 7.

### Formation of a PGA-AEEA(14)-Alfa-Coated Polyplex Particles

The PGA-AEEA(14)-Alfa shell was added by first mixing the appropriate amount of a solution of the PGA-conjugate in water with 1×HBT, followed by addition of the core-polyplex dispersion. The ratio of negatively charged glutamate residues of the PGA to positively charged nitrogens of the cationic polymer (“Glu/N ratio”) was found to be a suitable measure of PGA loading. With increasing Glu/N, the Zeta potential of preparations drops, and at Glu/N≥0.16, approaches the neutral zone (broadly defined as +/−15 mV); at the same time, increasing amounts of free cargo and free PGA-conjugate can be observed. Accordingly, PGA-AEEA(14)-Alfa polyplexes with a Glu/N between 0.01 and 0.04 were prepared.

### Functionalization and Testing of PGA-AEEA(14)-Alfa-Coated Polyplexes.

Ligand-functionalized particles were prepared by first mixing the appropriate amount of ligand (aCD3VHH×NbAlfa) and 1×HBT buffer and then adding a dispersion of PGA-AEEA(14)-Alfa-coated polyplexes. To ensure that no unbound ligand remains in solution, the molar ratio of Alfa-tag to ligand (“X:L ratio”) was always kept with an excess of Alfa tag; in this example, X:L ratios between 1.5 and 6.0 were tested in addition to an unfunctionalized reference (“X:L=0”). The volume of added buffer was chosen to result in a final cargo (RNA+DNA) concentration of 0.1 g/L.

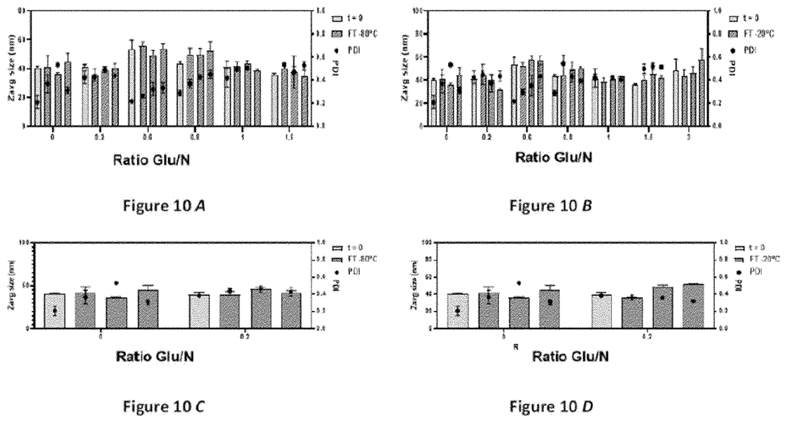

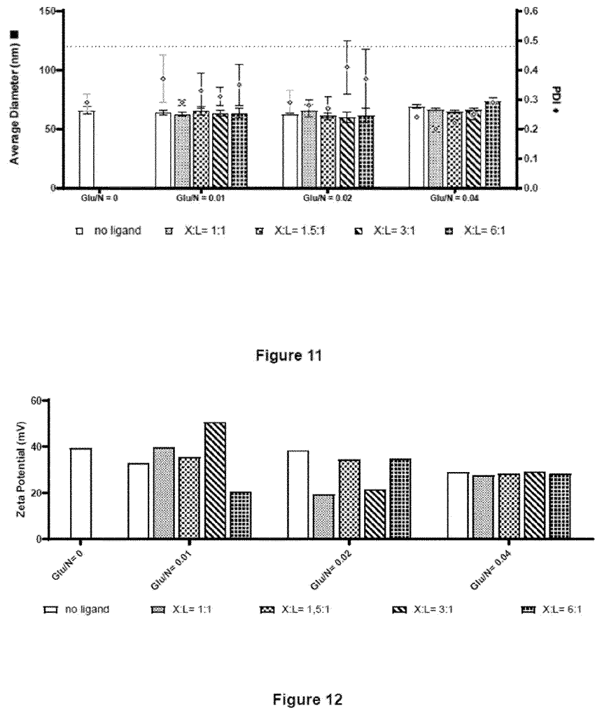

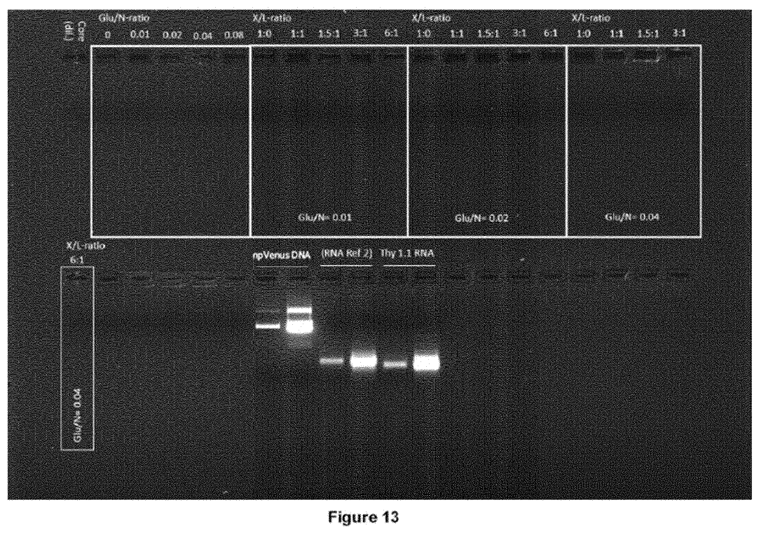

At low PGA loading (Glu/N=0.01-0.04), and across the tested ligand loadings, the size of the core particle is maintained; only at the highest measured Glu/N ratio, the size generally rises and shows more dependency on the amount of attached ligand (FIG. 11). The Pdl and the zeta potential drops with increasing PGA loading in the tested range (FIGS. 11-12). No free RNA or DNA was observed for any of the tested formulations (FIG. 13).

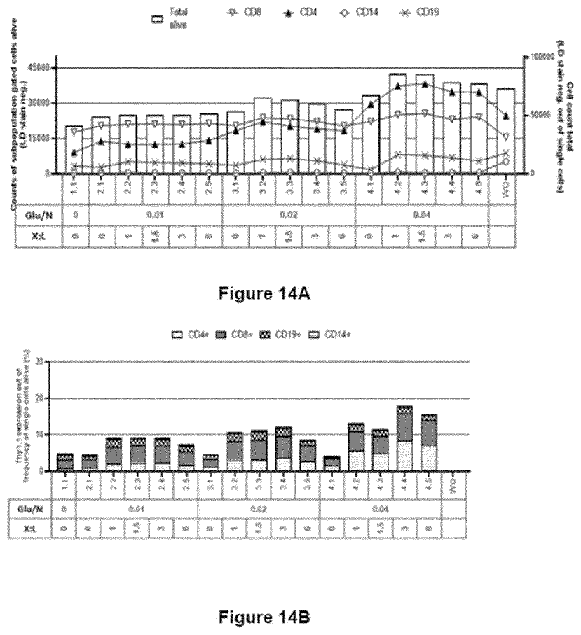

In vitro, the core particle and alfa-AEEA(14)-PGA PLX with low PGA loading show a decreased cell count, compared to the untreated reference, indicating toxicity. At higher PGA loading this toxicity is not only mitigated, but cells counts are elevated over the untreated reference, likely due to induction of proliferation by the CD3 ligand present on the particle surface (FIGS. 14A-B).

Ligand-mediated selective transfection of primary T-cells became apparent by the difference in transfection between non-functionalized and functionalized particles in the PBMC assay (FIGS. 14A-B). Surprisingly, the transfection efficiency was mostly governed by the amount of PGA-AEEA(14)-Alfa present, rather than the attached ligand, within the measured range. (FIGS. 14A-B).

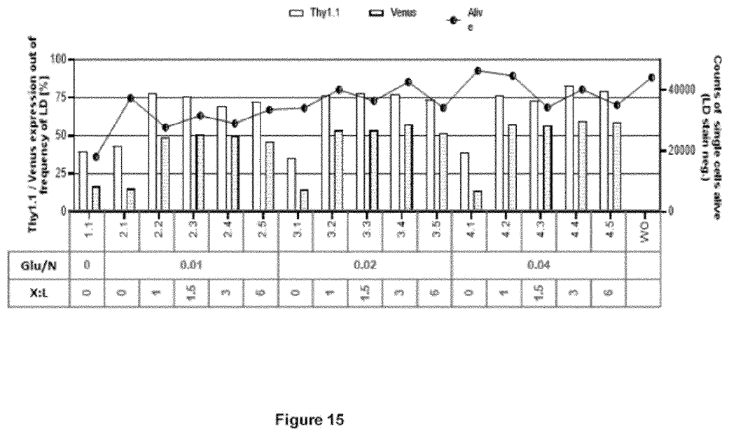

On Jurkat cells, similar findings were obtained for Thy-1.1 and Venus, with the difference that transfection of the non-functionalized and core formulations is generally higher (FIG. 15).

### Example 7

The present example evaluates linear and branched PEI to form binary complexes with modRNA and saRNA.

Preparation of Binary Core Particles with Linear and Branched PEI

A binary formulation consisting of Luciferase saRNA or Luciferase modRNA was complexed at N/P12 with linear PEI 500 (H[C2H4NH·HCl]500OH Mw 54.960 kDa) and branched PEI500 by mixing the two phases at 24 to 1 mixing ratio (RNA to Polymer phase). Binary formulation, or core polyplexes (PLXs), were manufactured at RNA final concentration of 0.125 mg/mL in a 1×MBG Buffer (final concentration 5% w/v Glucose, 10 mM MES, pH 6.0).

### Characterization of Binary Particles

Physicochemical characterization of binary saRNA-PLXs and modRNA-PLXs includes Zavg size and polydispersity index (PDI) using Wyatt DLS. Free RNA content was measured by agarose gel electrophoresis (AGE). Frozen stability studies were carried out by measuring particle size distribution using Wyatt for three consecutive freeze-thaw cycles. In those cycles, particles were frozen at either −20° C. or −80° C. overnight, thawed at room temperature for 1 hour, measured by standard Wyatt protocol and frozen again at the same temperature. This procedure was repeated three times, unless large size increases are observed.

Monomeric RNA was evaluated with a centrifugation and RNA quantification method. Briefly, 400 μl of binary PLX is filled in a 1.5 mL Eppendorf® tube and centrifuged at 20 000 rcf, 4° C. for 1.5 h (accelerating slope 9, slowing 5). A supernatant of 250 μl was taken out of each centrifuged formulation. The RNA concentration of the supernatant (where monomeric RNA was present) was measured and put in relation to the overall RNA content, giving the monomeric RNA of the formulation as a percentage of the total RNA. This method is comparable to an Ultracentrifugation assay where samples are measured with analytic ultracentrifuge and light absorption is measured at two different wavelengths: 255 nm and 650 nm. MBG sample buffer was used as blank sample for the measurements. Samples were centrifuged at 80.000 G at RT.

RNA concentration was quantified with Nanodrop. The microvolume option was used for the quantification of RNA—1.5 μl of formulated RNA was used. For the calculation of the RNA concentration, 260/280 and 260/230 nm purity ratios were considered, and formulation buffer was used as blanking solution for the RNA quantification of each sample.

Luciferase assays are a reliable and sensitive method to detect the expression of luciferase in live cells. In this assay, firefly luciferase catalyzes the mono-oxygenation of beetle luciferin via ATP and Mg2+-dependent. This results in emission of light in the range of 550 to 620 nm. For cell transfections, C2C12 and RAW 264.7 cells were seeded at respective cell number of 5,000 cells/well and 25,000 cells/well in Nunc white flat-bottom 96-well plate (Merck KGaA, Darmstadt, Germany) and centrifuged at 500×g for 5 min. After 18-24 hours, the medium was replaced by fresh medium at 90 μl/well; DPBS and only medium were used as negative and blank control, respectively. Formulation samples were tested at mRNA assay concentration between 12.5 to 100 ng per well. Samples and controls were added at 10 μl/well. After 24 hours, luciferase expression was determined by Bright-Glo™ Luciferase assay (Promega, Madison, WI, USA) according to manufacturer's protocol. The reagent was added to the cells in medium in 1:1 (v/v) ratio, followed by an incubation of 5 min in the dark to allow for complete cell lysis.

Viability was measured by CellTiter-Glo® (Promega, Madison, WI, USA) according to manufacturer's protocol. The reagent was added to the cells in medium in 1:1 (v/v) ratio, followed by an incubation of 10 minutes in a shaker followed by 20 minutes to allow for stabilization of the signal. Alternatively, luciferase and viability were determined by ONE-Glo™+Tox Luciferase Reporter and Cell Viability Assay (Promega GmbH, Madison, WI, USA) according to manual instructions: 20 μl of 5X CellTiter-Fluor™ Reagent was added to the wells, and mixed by orbital shaking (300-500 rpm for ˜30 sec); after incubation for 30 min at 37° C., the fluorescence was measured with excitation wavelength at 400 nm and emission at 505 nm (viability). Then, ONE-Glo™ Reagent was added at 100 μl/well. After incubation for three minutes, the bioluminescence was measured (luciferase expression). Bioluminescence signals (photons per second [p/s])) and fluorescence were measured using a microplate luminescence reader Infinite M200 (Tecan, Männedorf, Switzerland). Relative luminescence was calculated by subtracting the signal of DPBS control from the sample control. Relative viability was calculated by:

Viability %=[RLUsample/RLUcells only]×100

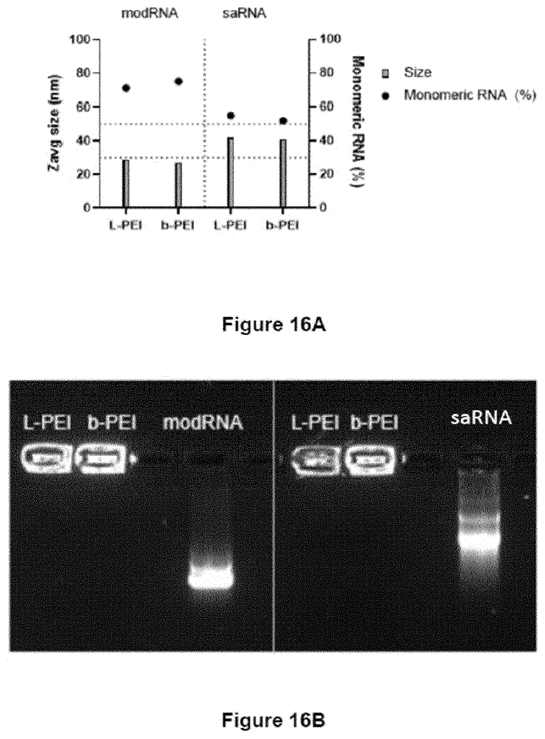

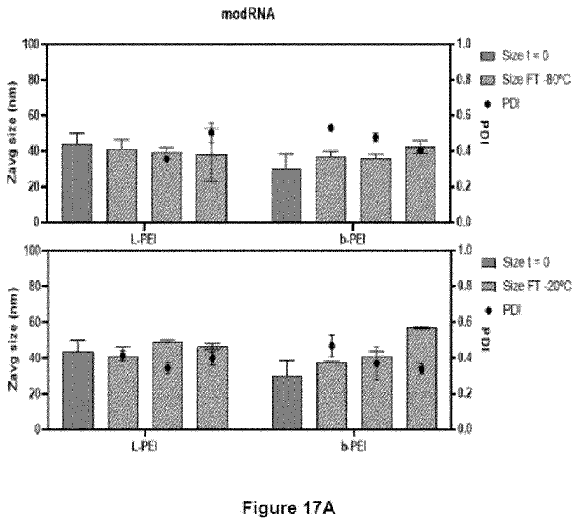

Binary saRNA-PEI PLXs present a size and monomeric RNA content of around 40 nm and 40% of the total RNA, respectively (FIG. 16A). Binary modRNA-PEI PLXs present a size of 30 nm and a monomeric RNA content of around 75%, respectively (FIG. 16A). No free RNA is detected using Agarose gel electrophoresis (AGE) (FIG. 16B). PLXs are freezable and no size increase is observed at −80° C. and −20° C. for at least 3 freeze thaw cycles (FIGS. 17A and 17B).

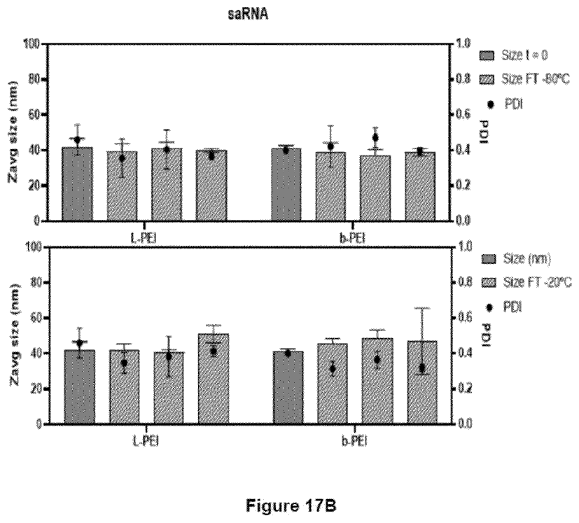

In vitro expression was evaluated in C2C12, RAW and HEPG2 cell lines. modRNA-PEI and saRNA-PEI polyplexes were evaluated at 4 different RNA concentrations: 100 ng, 50 ng, 25 ng and 12.5 ng of RNA per well. Results show that for both modRNA and saRNA, polyplexes prepared with linear PEI (L-PEI) show a luciferase signal systematically higher than polyplexes prepared using branched PEI (b-PEI) (FIGS. 18A-C, 19A-C, 20A-C, and 21A-C), confirming the higher performance in vitro of the linear cationic polymer over the branched cationic polymer.

### Example 8

The present example describes preparation and characterization of certain complexes, including at least those used in Example 9-17, although they can be applied to various complexes described throughout the present application. As described herein, reference to a “binary formulation” or “binary particle” is also intended to refer to a “core complex” as described throughout the present application. That is, a binary formulation comprises a cationic polymer and a nucleic acid. A “ternary formulation” or “ternary particle” is also intended to refer to a complex comprising a cationic polymer, a nucleic acid, and an anionic polymer, wherein the cationic polymer and the nucleic acid form a core complex that is encapsulated by an anionic polymer.

### Preparation of Binary Core Particles

A binary formulation consisting of Luciferase saRNA was complexed at N/P12 with linear PEI 500 (H[C2H4NH·HCl]500OH Mw 54.960 kDa). Manufacturing was carried out using a fluid path process by mixing RNA and Polymer phases. Binary formulation, or core polyplexes (PLXs), were manufactured at RNA final concentration of 0.1 mg/mL in a 1×MBG Buffer (final concentration 5% w/v Glucose, 10 mM MES, pH 6.0). These binary saRNA-PEI PLXs present a size of around 35 nm and a strong positive zeta potential of 20 to 25 mV. No free RNA is detected using Agarose gel electrophoresis (AGE). PLXs are freezable and no size increase is observed at −80° C. for at least 3 freeze thaw cycles. Binary saRNA complexes present a monomeric RNA content of 40% of the total RNA.

A binary formulation consisting of Luciferase modRNA was complexed at N/P12 with linear PEI 500 (H[C2H4NH·HCl]500OH) with Mw 54.960 kDa). Manufacturing was carried out using a fluid path process by mixing RNA and Polymer phases. The binary formulation (also referred to herein as or core complexes or core polyplexes (PLXs)), were manufactured to an RNA final concentration of 0.125 mg/mL in a 1×MBG Buffer (final concentration 5% w/v Glucose, 10 mM MES, pH 6.0). These binary modRNA-PEI PLXs present a size measured by Wyatt of around 30 nm and a strong positive zeta potential of 20 to 25 mV. No free RNA is detected using Agarose gel electrophoresis (AGE). PLXs were freezable and no size increase was observed after thawing at −80° C. Binary modRNA complexes present high monomeric RNA content, typically 80% of total RNA is in monomeric form.

### Preparation of Ternary Complexes

The formation of ternary formulations of modRNA were produced after addition of the anionic PGA-based polymer on the binary formulations (core polyplexes). The ratio of negatively charged glutamate residues of the PGA to positively charged nitrogen atoms of the cationic polymer (“Glu/N ratio”) was found to be a suitable measure of PGA loading. The formation of ternary complexes was studied within a range of Glu/N charge ratio from 0 to 3. After addition of the appropriate amount of each anionic polymer, the concentration was adjusted to 0.10 mg/ml RNA in all samples and final storage matrix was 1×MBG Buffer (5% w/v Glucose, 10 mM MES, pH 6.0).

The formation of ternary formulations of modRNA were produced after addition of a homopolymer of n-butyl-Poly-(L-Aspartic acid sodium salt) 50 (PAsp). The ratio of negatively charged aspartate residues of the PAsp to positively charged nitrogen atoms of the cationic polymer (“Asp/N ratio”) was found to be a suitable measure of PAsp loading The formation of ternary complexes was studied within a range of Asp:N charge ratio from 0 to 3. After addition of the appropriate amount of the anionic polymer, the concentration was adjusted to 0.10 mg/ml RNA in all samples and final storage matrix is 1×MBG Buffer (5% w/v Glucose, 10 mM MES, pH 6.0).

The ternary formulations of modRNA were produced by mixing of RNA, cationic polymer and anionic polymer to form the core-shell complexes. Final buffer composition consists of 1×MBG Buffer (final concentration 5% w/v Glucose, 10 mM MES, pH 6.0).

The formation of ternary formulations of saRNA were produced after addition of the anionic PGA-based polymer on the binary formulations (core polyplexes). The ratio of negatively charged glutamate residues of the PGA to positively charged nitrogens of the cationic polymer (“Glu/N ratio”) was found to be a suitable measure of PGA loading. The formation of ternary complexes is studied within a range of Glu/N charge ratio from 0 to 3. After addition of the appropriate amount of each anion, the concentration is adjusted to 0.08 mg/ml RNA in all samples and MBG Buffer (final concentration 5% w/v Glucose, 10 mM MES, pH 6.0).

### Characterization of Binary and Ternary Particles

Physicochemical characterization of binary and ternary saRNA-PLXs and modRNA-PLXs includes Zavg size and polydispersity index (PDI) using Wyatt DLS. Surface charge zeta potential was measured using Malvern Zetasizer. Variations of zeta potential, as a consequence of the change in the charge exposed by the particles surface, further confirm the systematic coating of core polyplexes with the anionic polymer. Free RNA content was measured by agarose gel electrophoresis (AGE). Frozen stability studies are carried out by measuring particle size distribution using Wyatt for three consecutive freeze-thaw cycles. In those cycles, particles are frozen at either −20° C. or −80° C. overnight, thawed at room temperature for 1 hour, measured by standard Wyatt protocol and frozen again at the same temperature. This procedure was repeated three times.

Monomeric RNA was evaluated with a centrifugation and RNA quantification method. Briefly, 400 μl of binary PLX is filled in a 1.5 mL Eppendorf® tube and centrifuged at 20 000 rcf, 4° C. for 1.5 h (accelerating slope 9, slowing 5). A supernatant of 250 μl was taken out of each centrifuged formulation. The RNA concentration of the supernatant (where monomeric RNA is present) is measured and put in relation to the overall RNA content, giving the Monomeric RNA of the formulation as a percentage of the total RNA. This method is comparable to an Ultracentrifugation assay where samples are measured with analytic ultracentrifuge and light absorption is measured at two different wavelengths: 255 nm and 650 nm. MBG sample buffer was used as blank sample for the measurements. Samples are centrifuged at 80.000 G at RT.

An example of a typical result for modRNA PEI-PLXs was shown in the table below. In this example, to derive the monomeric RNA content of the formulation, the ratio of the RNA concentration from the centrifuged PLXs over the uncentrifuged one is considered, and this ratio gives a result of 70% monomeric RNA content.

Results from Monomeric RNA Assay for modRNA PEI-PLXs

Results from monomeric RNA assay for modRNA PEI-PLXs

| Sample | RNA concentration (mg/mL) |
|---|---|
| Centrifuged modRNA PEI-PLX | 0.804 |
| Uncentrifuged modRNA PEI-PLX | 0.1165 |

RNA concentration was quantified with Nanodrop. The microvolume option was used for the quantification of RNA, more specifically 1.5 μl of formulated RNA are used. For the calculation of the RNA concentration, 260/280 and 260/230 nm purity ratios are considered, and formulation buffer is used as blanking solution for the RNA quantification of each sample.

Both modRNA and saRNA protection against RNAses can be evaluated by serum incubation, as those types of RNA degrade very promptly in serum. Human serum solution was prepared to dilute the samples in MBG5% (D-Glucose 5% w/v, 10 mM MES, pH 6.0) to a final concentration of 0.05 mg/mL and 10% v/v human serum. Negative controls are incubated in MBG 5%. The samples were incubated at 37° C. for 30 minutes and afterwards cooled in a 4° C. bath for 10 minutes. For the preparation of the agarose gel, 100 ml of 1% Agarose in TAE Buffer was used. 10 μl of GelRed was added prior gel solidification for RNA fluorescent labeling. Each sample containing 1 μg of RNA was diluted with gel loading buffer to a final volume 15 of 24 μl that is loaded into each well of the agarose gel. The samples were run at 80V, 50 mA for 40 min. UV-fluorescence was measured after 0.5 seconds of exposure time to enhance the visibility of faint bands.

Luciferase assays are a reliable and sensitive method to detect the expression of luciferase in live cells. In this assay, firefly luciferase catalyzes the mono-oxygenation of beetle luciferin via ATP and Mg2+-dependent. This results in emission of light in the range of 550 to 620 nm. For cell transfections, C2C12 and RAW 264.7 cells were seeded at respective cell number of 5,000 cells/well and 25,000 cells/well in Nunc white flat-bottom 96-well plate (Merck KGaA, Darmstadt, Germany) and centrifuged at 500×g for 5 min. After 18-24 hours, the medium was replaced by fresh medium at 90 μl/well; DPBS and only medium were used as negative and blank control, respectively. Formulation samples were tested at mRNA assay concentration between 12.5 to 100 ng per well. Samples and controls were added at 10 μl/well. After 24 hours, luciferase expression was determined by Bright-Glo™ Luciferase assay (Promega, Madison, WI, USA) according to manufacturer's protocol, that is, the reagent was added to the cells in medium in 1:1 (v/v) ratio, followed by an incubation of 5 min in the dark to allow for complete cell lysis. Viability was measured by CellTiter-Glo® (Promega, Madison, WI, USA) according to manufacturer's protocol, that is, the reagent was added to the cells in medium in 1:1 (v/v) ratio, followed by an incubation of 10 min in shaker followed by 20 min to allow for stabilization the signal. Alternatively, luciferase and viability were determined by ONE-Glo™+Tox Luciferase Reporter and Cell Viability Assay (Promega GmbH, Madison, WI, USA) according to manual instructions. Briefly, 20 μl of 5X CellTiter-Fluor™ Reagent was added to the wells, mix by orbital shaking (300-500 rpm for ˜30 sec). After incubation for 30 minutes at 37° C., the fluorescence was measured with excitation wavelength at 400 nm and emission at 505 nm (viability). Then, ONE-Glo™ Reagent was added at 100 μl/well. After incubation for three min, the bioluminescence was measured (luciferase expression). Bioluminescence signals (photons per second [p/s])) and fluorescence were measured using a microplate luminescence reader Infinite M200 (Tecan, Männedorf, Switzerland). Relative luminescence was calculated by subtracting the signal of DPBS control from the sample control. Relative viability was calculated by:

$$Viability \% = [ RLUsample / RLUcells only ] \times 100$$

### Example 9

The present example provides an evaluation of homopolymer poly-α-L-glutamic acid length to form ternary complexes with modRNA-PEI.

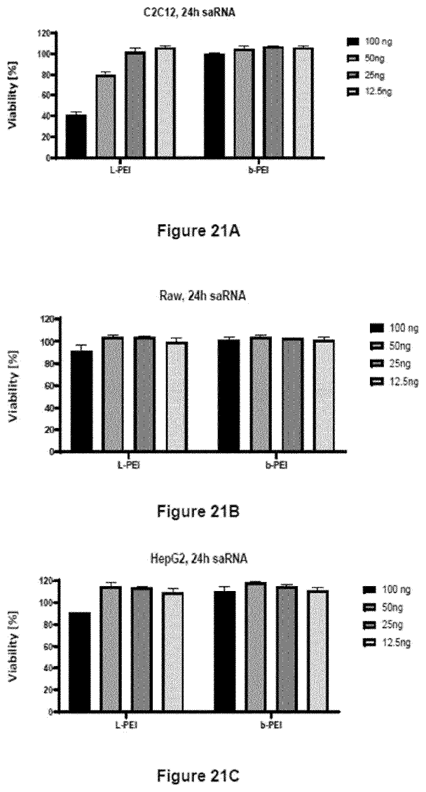

Physicochemical characterization shows that ternary polyplexes can be formulated when PGA with different monomeric units is used, with sizes ranging between 50 nm and 150 nm. Ternary complexes sizes are dependent on Glu/N ratio and PGA length. As a general trend, ternary PLXs are larger for Glu/N ratios close to neutral surface charge (ratios 0.4 to 0.8), while particle size remains relatively small in either very small (0.2) or very large (3) Glu/N ratios (Zavg≤100 nm) (FIG. 22). PGA length affects both size and PDI. PGA(50) and PGA(20) lead to low complexes sizes among all the Glu/N tested (Zavg≤150 nm). At the same time the shortest PGA PGA(20) lead to the most uniform particles sizes distribution, with PDI≤0.3. Surface charge of particles can be tuned with increasing amounts of anionic polymer added on core polyplexes and it completely shifts from approximately +25 mV to −25 mV as seen from zeta potential values in FIG. 22. Variation of zeta potential, as a consequence of the change in the charge exposed by the particles surface, further confirm the systematic coating of core polyplexes with the anionic polymer.

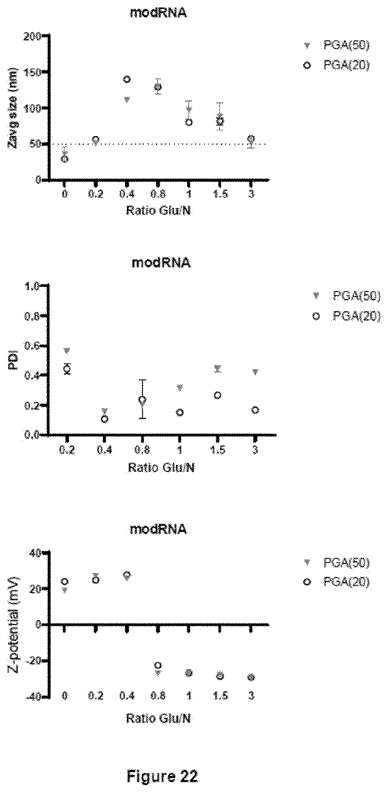

Free RNA content was measured with AGE and showed in FIG. 23. For PGA(20) and PGA(50), no free RNA was detected in any of the Glu/N tested, suggesting a successful encapsulation of the cargo.

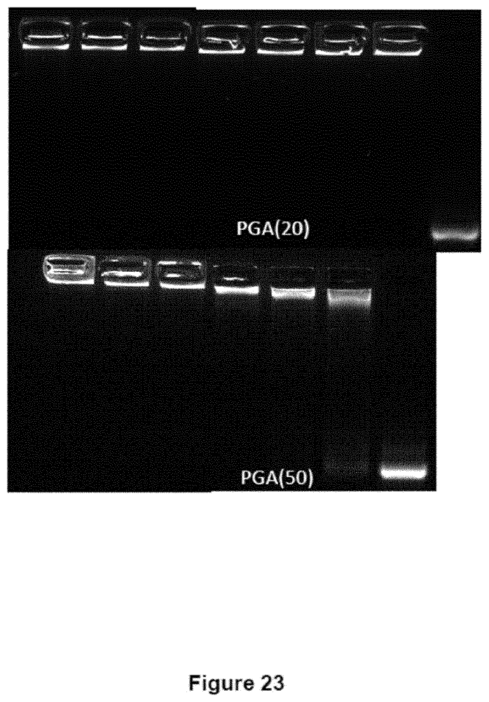

Ternary complexes were tested for release of the cargo and RNAse degradation upon serum incubation. FIG. 24 shows corresponding AGE images of all ternary complexes with the different PGA lengths and ratios including control group (untreated samples) and samples that undergo serum incubation. The compaction of the modRNA induced by the interaction with PEI successfully protects it against the RNases degradation. Moreover, for shorter PGA chains ((PGA(20)) no increase of free RNA content is observed upon serum incubation, while longer chains (PGA(50)) show the presence of free RNA starting from Glu/N ratio 0.8.

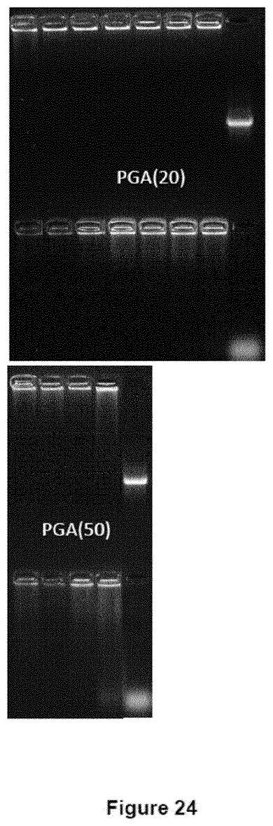

FIG. 25 shows that PGA coating imparts colloidal stability upon freeze thawing at both −20° C. and −80° C. and that the length of the polymer affects the Glu/N range in which stability is achieved.

### Example 10

The present examples describes evaluation of homopolymer of n-butyl-Poly-(L-Aspartic acid) to form ternary complexes with modRNA-PEI.

Poly-(L-Aspartic acid) (PAsp) was tested as polymer coating of core polyplexes because of its desirable properties for the development of a new drug delivery carrier. PAsp is in fact biocompatible, biodegradable and show low cytotoxicity. See Nie, et al., ACS Appl. Mater. Interfaces 2015, 7, 1, 553-562.

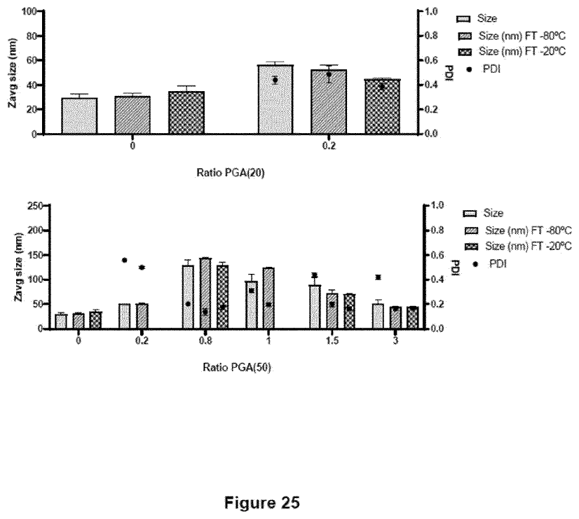

Physicochemical characterization is illustrated in FIG. 26. Ternary complexes can be formulated with PAsp obtaining particles sizes in the 50-150 nm range as a function of the Asp:N used. Ternary PLXs show large particle size distribution for ratios close to neutral surface charge (ratio 0.6), while particle size remains relatively small in either very small (0.2) or very large (3) Asp:N ratios (FIG. 26). Very low and very high Asp:N ratios lead to the least uniform particles sizes distribution (PDI>0.3 for Asp:N 0.2 and ≥1.5), while more homogenous complexes distribution are obtained for Asp:N ratios in the 0.4-0.6 range. Surface charge of particles can be tuned with increasing amounts of anionic polymer added on core polyplexes and it completely shifts from approximately +25 mV to −25 mV as seen from zeta potential values in FIG. 26. Variation of zeta potential, as a consequence of the change in the charge exposed by the particles surface, further confirms the systematic coating of core polyplexes with the anionic polymer.

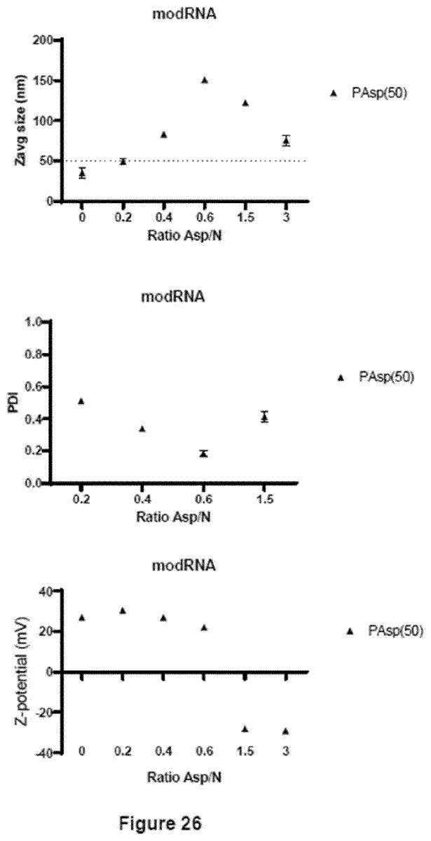

Free RNA content was measured with AGE (FIG. 27) and images show that it is possible to obtain ternary complexes coating PEI-RNA with a layer of biodegradable polymer like PAsp with acceptable size and PDI (Sizes≤150 nm and PDI≤0.3), and that the amount of biodegradable polymer can be modulated on the core polyplexes surface to obtain complexes with different surface charges without altering the polyplex structure (no free RNA up to Asp:N 0.6).

### Example 11

The present example describes an evaluation of block-copolymer of Poly-α-L-glutamic acid-block-poly-N-methyl-sarcosine length to form ternary complexes with modRNA-PEI, including an investigation of stability in serum and in vitro transfection activity.

The formation of ternary formulations of modRNA was produced after addition of a block-copolymer of Poly-α-L-glutamic acid-block-poly-N-methyl-sarcosine (PGA(50)-b-pSar(X), with X=10, 25, 50 repetitive units).

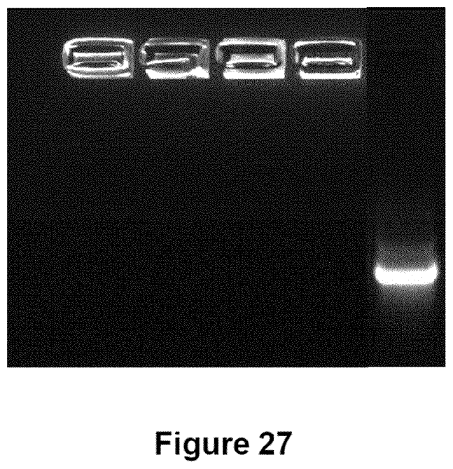

Physicochemical characterization shows that a size decrease was observed in all cases with the increase in length of the pSar (FIG. 28). Complexes prepared with PGA(50)-b-pSar(50) showed the smallest sizes˜50 nm across all the Glu/N tested.

Additionally, there is a general decrease in PDI with increasing pSar length, namely particle size distributions are more homogeneous (FIG. 28). As the pSar length increases, the particles become more stable. Without wishing to be bound by theory it is hypothesized that stability increases as the steric repulsion interactions between the particles become stronger. Addition of increasing amounts of PGA(50)-b-pSar(50) cause the size distribution of ternary formulations to remain almost constant, from 50 to 60 nm, while the surface charge systematically changes from +25 to −25 mV.

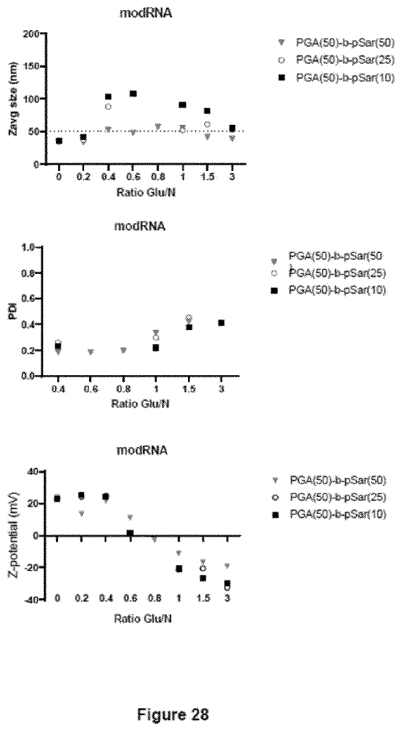

Free RNA content was measured with AGE and showed in FIG. 29. In this case, no significant differences emerge with increasing pSar length in terms of free RNA detected, In this case, no significant differences emerge with increasing pSar length in terms of free RNA were detected, as in all cases a good encapsulation of the cargo is obtained up to Glu/N of 0.6.

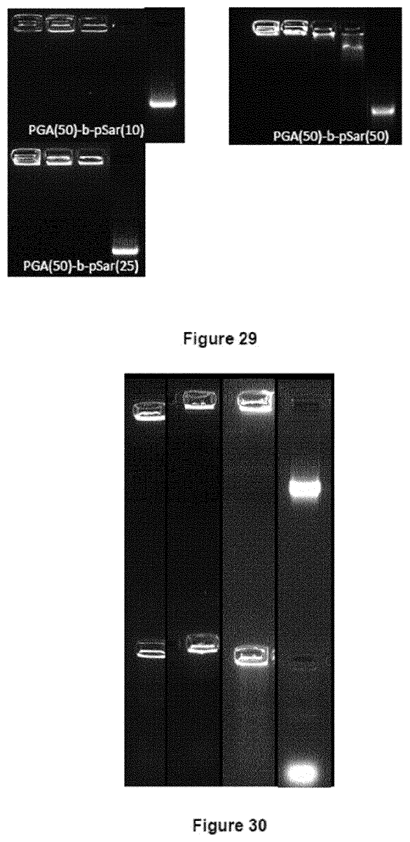

Ternary complexes formed with PGA(50)-b-pSar(50) at Glu:N 0.6 were tested for release of the cargo upon serum incubation for 30 min, 1 h and 2 h (FIG. 30). No increase of free RNA content is observed upon serum incubation up to 2 h, which indicates that PGA(50)-b-pSar(50) can successfully protect polyplexes from serum proteins destabilization.

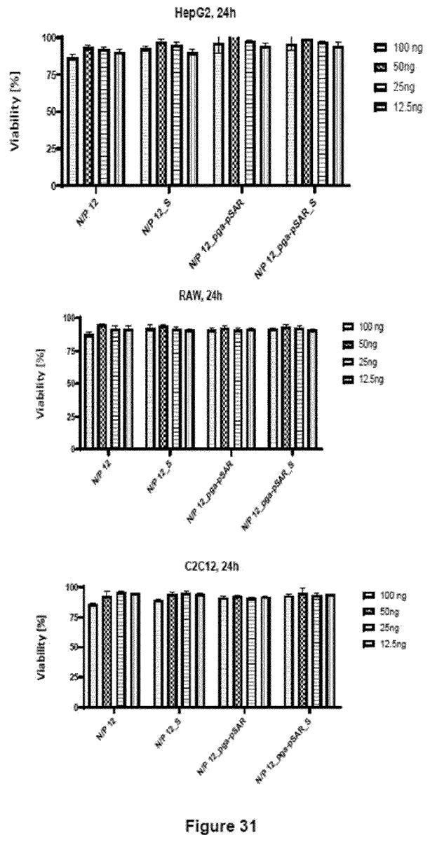

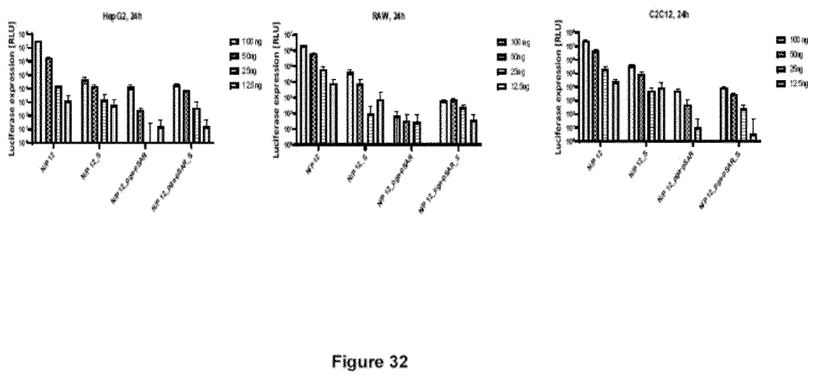

In vitro expression was evaluated in C2C12, RAW and HEPG2 cell lines. Ternary modRNA-PEI polyplexes complexed with PGA(50)-b-pSar(50) at 0.6 Glu/N ratio were evaluated at 4 different RNA concentrations, namely 100 ng, 50 ng, 25 ng and 12.5 ng of RNA per well before and after 2 hours of serum incubation. Control samples, core polyplexes with no coating, were included. Results show cell viability is maintained upon coating of core polyplexes with PGA(50)-bpSar(50) (FIG. 31). Most importantly, data show that upon serum incubation, core polyplexes have a decrease in luciferase expression, while coated complexes can successfully maintain luciferase expression, particularly at low doses, in all the cell lines tested (FIG. 32).

### Example 12

The present example describes evaluation of Alfa tagged block-copolymer of Poly-α-L-glutamic acid-block-poly-N-methyl-sarcosine, Poly-α-L-glutamic acid and Poly-α-L-glutamic acid-block-poly-N— Ac-AEEA length to form ternary complexes with modRNA-PEI polyplexes.

The formation of ternary formulations of modRNA are produced after addition of either Alfa tagged block-copolymer of Poly-α-L-glutamic acid-block-poly-N-methyl-sarcosine (PGA(50)-b-pSar(50)-Alfa), an Alfa tagged homopolymer of Poly-α-L-glutamic acid (PGA(X)-Alfa, with X=50, 100 repetitive units) or an alfa tagged block-copolymer of Poly-α-L-glutamic acid-block-poly-N— Ac-AEEA (PGA(50)-b-Ac-AEEA(14)-Alfa).

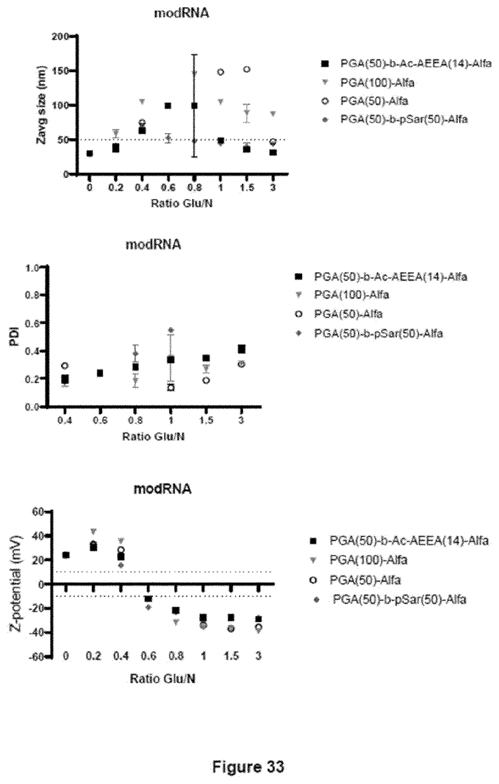

Ternary complexes with PGA(50)-b-pSar(50)-Alfa, PGA(50)-b-Ac-AEEA(14)-Alfa, PGA(50)-Alfa and PGA(100)-Alfa can be successfully formed with sizes being a function of the polymer used and the Glu/N ratio tested (FIG. 33, top graph). The presence of a stealth moiety as a linker between PGA and Alfa peptide (pSar(50) and pAEEA(14)) enables to maintain Zavg sizes below 100 nm. Across the majority of Glu/N tested, ternary complexes show acceptable distributions, with PDI values≤0.4 and high PDI are usually obtained at high Glu/N ratios (FIG. 33, middle graph). At the same time, surface charge of the particles can be tuned, as with increasing amounts of anionic polymer, zeta potential values completely shift from approximately +25 mV to −25 mV as seen in FIG. 33, bottom graph.

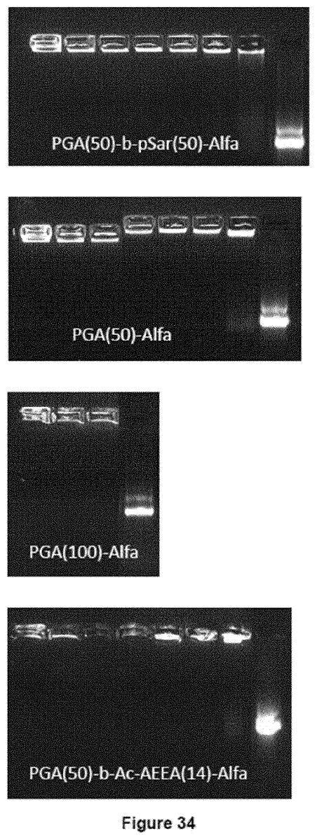

AGE analysis shows that Alfa-tagged modRNA ternary complexes can be formulated within certain Glu/N ratios achieving fully RNA encapsulation (FIG. 34).

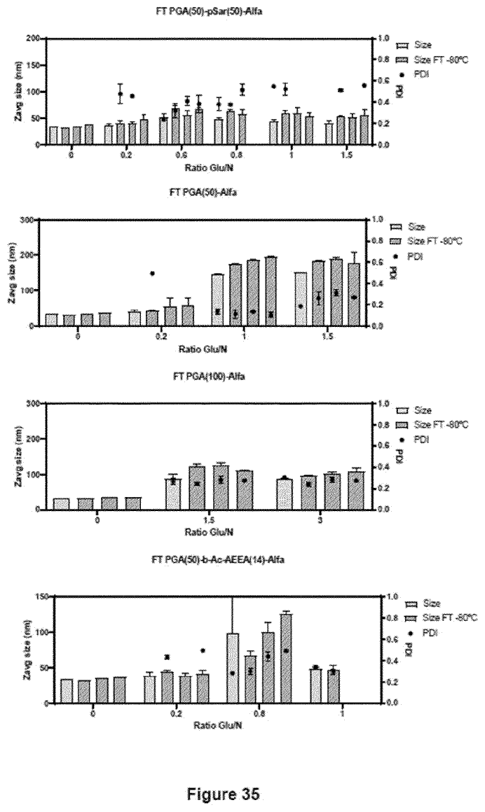

Ternary complexes coated with Alfa-tagged polymers can be successfully stabilized at both −80° C. and −20° C., up to three freeze thaw cycles and in a wide range of Glu/N ratios, thus ensuring frozen stability of particles positively, neutral and negatively charged (FIG. 35).

### Example 13

The present example describes evaluation of homopolymer poly-α-L-glutamic acid length to form ternary complexes with saRNA-PEI.

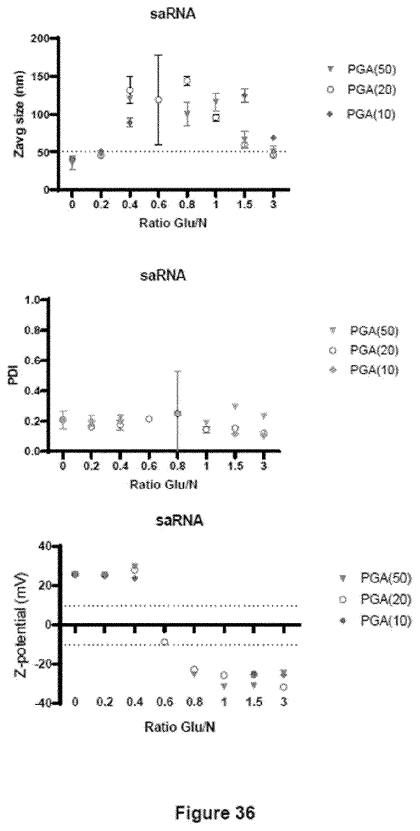

Physicochemical characterization shows that ternary complexes were obtained even when bigger RNA constructs are used, like in the case of saRNA, with sizes ranging between 50 nm and 150 nm. Ternary complexes sizes were impacted by Glu/N ratio tested and PGA length. It was found that ternary PLXs show larger particle sizes for Glu/N ratios close to neutral surface charge (ratios 0.6 to 0.8), while particle size remains smaller in either very small (0.2) or very large (3) Glu/N ratios (Zavg≤100 nm) (FIG. 36, top graph). PGA length affects mainly the uniformity of size distributions, with the longest PGAs (PGA(50) and (PGA(100)) leading to very homogenous complexes populations (PDI≤0.3) (FIG. 36, middle graph). Surface charge of particles can be tuned with increasing amounts of anionic polymer added on core polyplexes and it completely shifts from approximately +25 mV to −25 mV as seen from zeta potential values in FIG. 36, bottom graph.

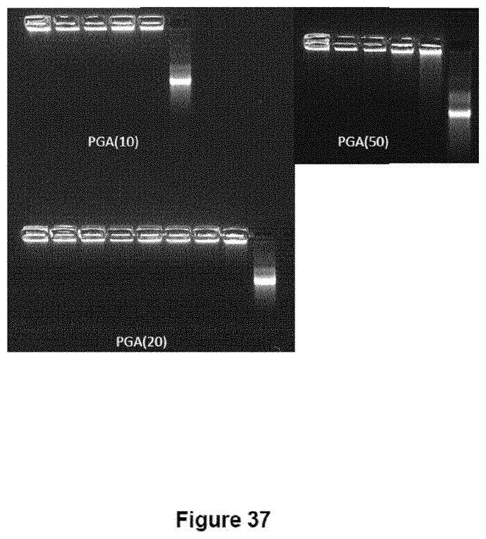

AGE results show that ternary complexes can be prepared achieving full cargo encapsulation across the Glu/N ratios tested (FIG. 37).

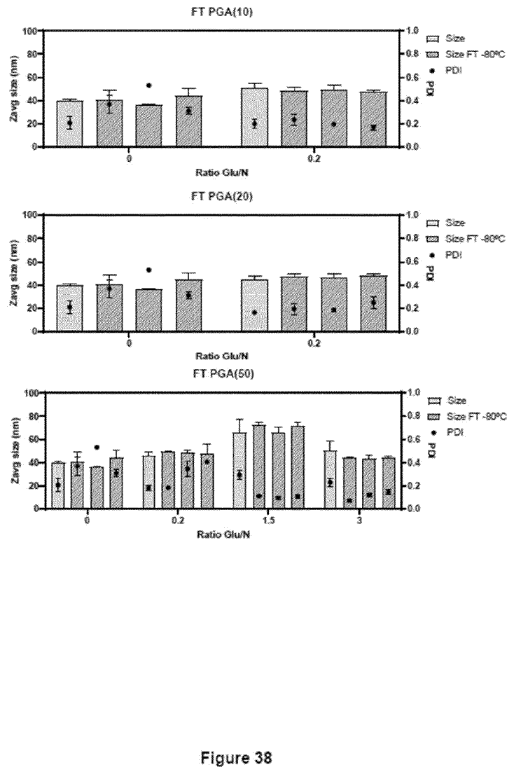

FIG. 38 shows that PGA coating can guarantee colloidal stability upon freeze thawing at both −20° C. and −80° C. and that the length of the polymer affects the Glu/N range in which stability is achieved. In this context, PGA(50) is particularly efficient in maintaining colloidal properties upon repeated freeze thawing in the widest range of Glu/N tested.

### Example 14

The present example describes evaluation of block-copolymer of Poly-α-L-glutamic acid-block-poly-N-methyl-sarcosine length to form ternary complexes with saRNA-PEI: investigation of in vitro transfection activity.

The formation of ternary formulations of saRNA were produced after addition of a block-copolymer of Poly-α-L-glutamic acid-block-poly-N-methyl-sarcosine (PGA(50)-b-pSar(X), with X=10, 25, 50 repetitive units).

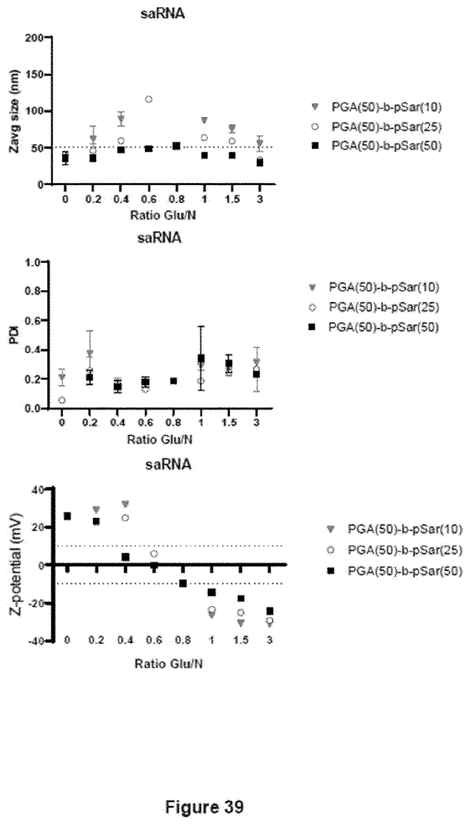

Physicochemical characterization shows that a size decrease is observed in all cases with the increase in length of the pSar, and this was especially significant in ratios where particles are close to neutral charge (0.6) (FIG. 39, top graph). Complexes prepared with PGA(50)-b-pSar(50) showed the smallest sizes˜50 nm across all the Glu/N tested. Ternary complexes had small size distribution at low Glu:N charge ratios (up to 0.8), with PDI≤0.3 (FIG. 39, middle graph). Addition of increasing amounts of PGA(50)-b-pSar(50) caused the size distribution of ternary formulations to remain at an almost constant size, from 50 to 60 nm, while the surface charge systematically changes from +25 to −25 mV.

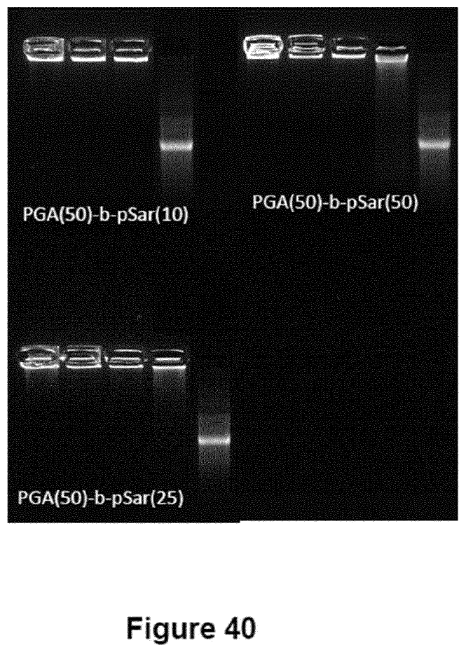

Free RNA content was measured with AGE and showed in FIG. 40. In this case, no significant differences emerge with increasing pSar length in terms of free RNA detected, as in all cases a good encapsulation of the cargo is obtained up to Glu/N of about 0.6.

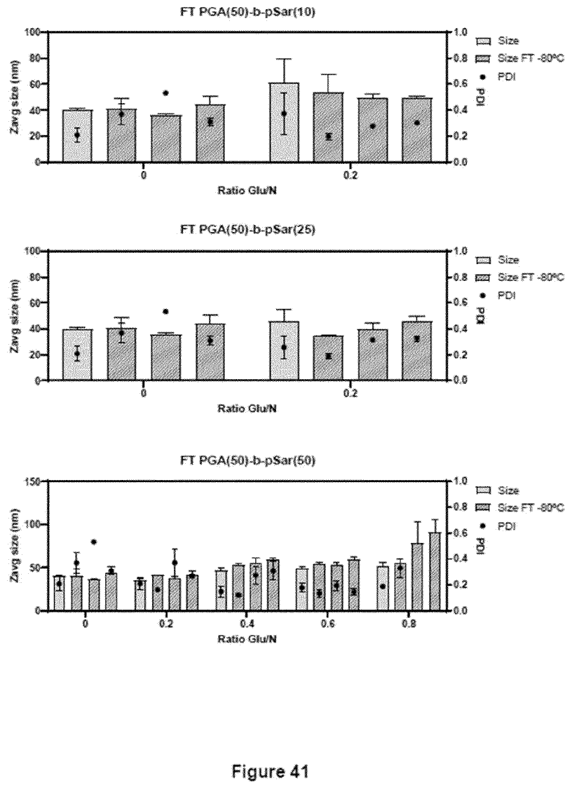

FIG. 41 shows that PGA coating can guarantee colloidal stability upon freeze thawing at −80° C. and that the length of the stealth moiety affects the Glu/N range in which stability is achieved. In this context, PGA(50)-b-pSar(50) is particularly efficient in maintaining colloidal properties upon repeated freeze thawing in the widest range of Glu/N tested.

### Example 15

The present example describes evaluation of homopolymer Alfa tagged poly-α-L-glutamic acid length to form ternary complexes with saRNA-PEI polyplexes.

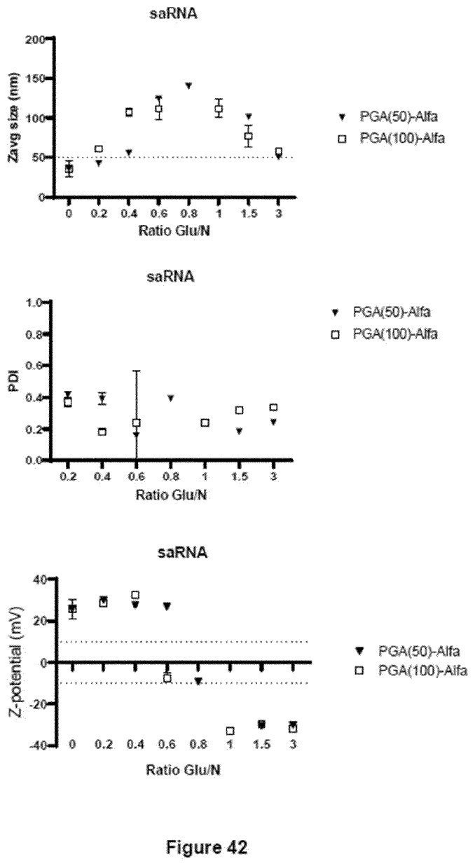

Physicochemical characterization showed that alfa-tagged ternary complexes can be formed with both PGA50-Alfa and PGA100-Alfa, in a size range between 40 and 150 nm, with bigger sizes obtained at Glu/N 0.4-1 (FIG. 42, top graph). Particle size remains relatively small in either very small (0.2) or very large (3) Glu/N ratios. PDI, shown in FIG. 42, middle graph, was about ≤0.4. Surface charge of the particles can be tuned with increasing amounts of ternary polymer and it completely shifts from approximately +25 mV to −30 mV as seen in FIG. 42, bottom graph.

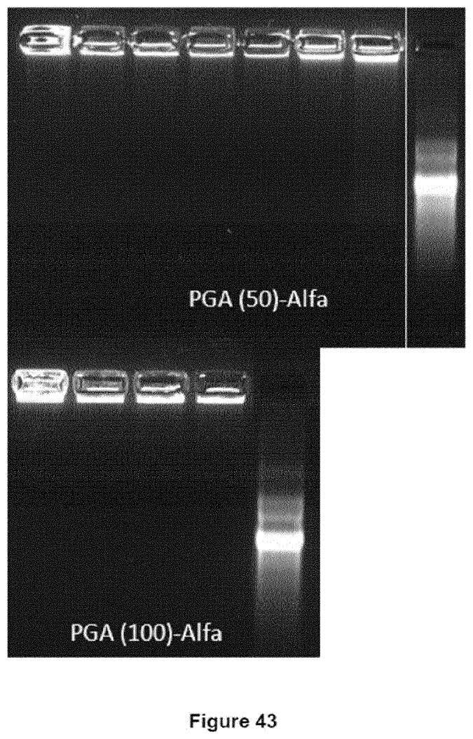

Agarose gel electrophoresis (AGE) analysis shows RNA can be successfully encapsulated up when saRNA binary polyplexes are coated with both PGA(50)-Alfa and PGA(100)-Alfa (FIG. 43).

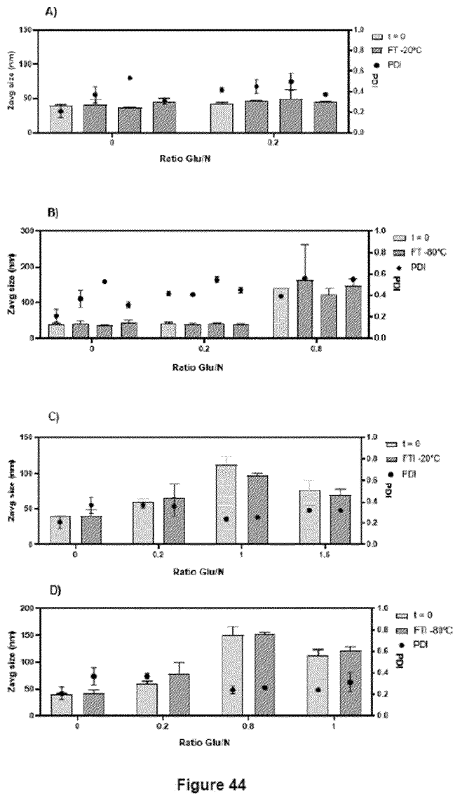

Frozen stability studies show that for PGA50-Alfa colloids are stable up to 3 freeze thaw cycles only at low amount of PGA (Glu/N 0.2), when particles are still highly positive charged. For the same polymer length, −80° C. stability can be achieved also at higher Glu/N ratios (0.8). Increasing the polymer length allows to achieve colloidal stability at −20° C. and −80° C. also for negatively charged complexes (Glu/N ratios of 1 and 1.5) (FIG. 44).

### Example 16

The present example describes evaluation of Alfa tagged block-copolymer of Poly-α-L-glutamic acid-block-poly-N-methyl-sarcosine and Poly-α-L-glutamic acid-block-poly-N—Ac-AEEA14 to form ternary complexes with saRNA-PEI polyplexes.

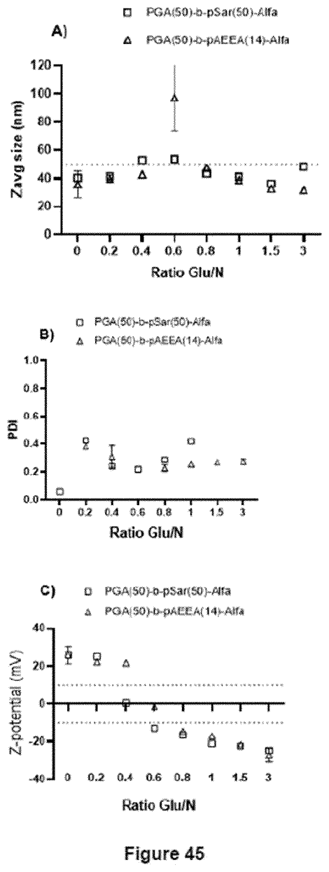

Physicochemical characterization showed that ternary complexes with PGA(50)-b-pSar(50)-Alfa and PGA(50)-b-Ac-AEEA(14)-Alfa can be successfully formed maintaining sizes between 40 and 60 nm (FIG. 45, top graph). In the majority of Glu/N ratios no significant differences are observed in terms of sizes between the two different stealth used (pSar(50) and pAEEA(14)). PDI values≤0.4 are obtained for both anionic polymers (FIG. 45, middle graph). These results show that when a stealth moiety is present as linker between the anion polymer and the alfa (pSar(50) and pAEEA(14)), the sizes of the ternary complexes can be maintained below 70 nm.

Surface charge of the particles can be tuned, as with increasing amounts of anionic polymer, zeta potential values completely shift from approximately +25 mV to −25 mV as seen in FIG. 45, bottom graph.

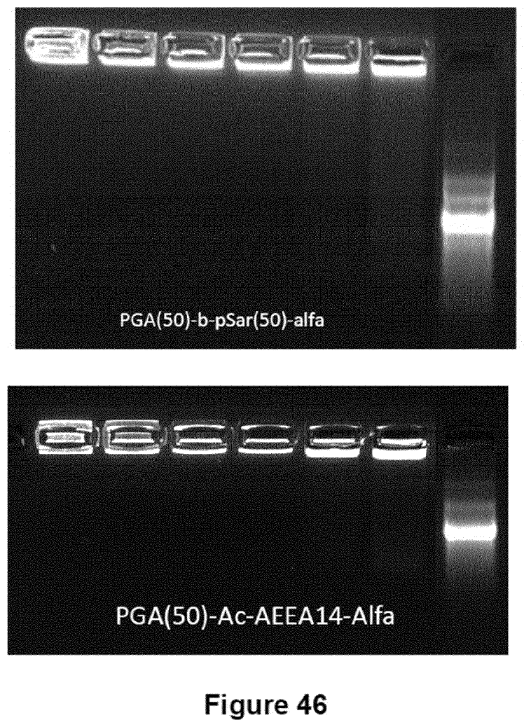

AGE results shows the saRNA can successfully encapsulated upon coating of binary polyplexes with PGA(50)-b-pSar(50)-Alfa and PGA(50)-b-Ac-AEEA(14)-Alfa (FIG. 46).

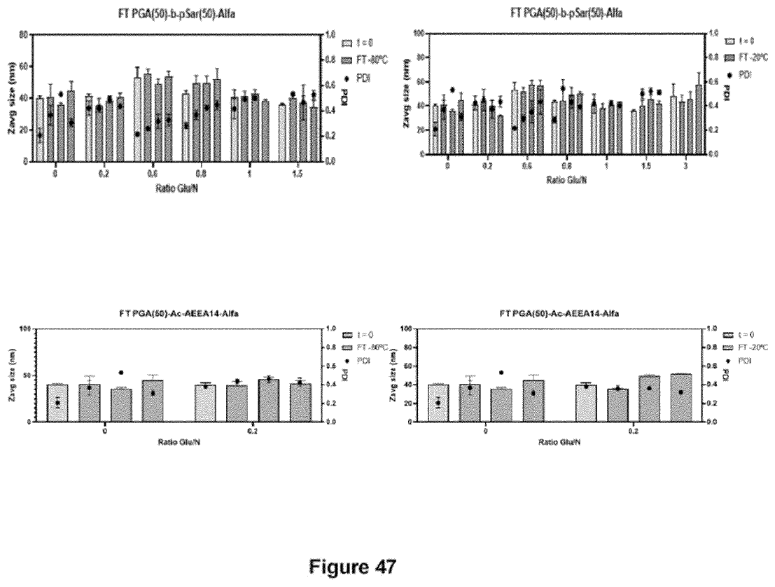

Ternary complexes coated with PGA(50)-b-pSar(50)-Alfa can be successfully stabilized at both −80° C. and −20° C., up to three freeze thaw cycles and in a broad range of Glu/N ratios, thus ensuring frozen stability of particles positively, neutral and negatively charged. Ternary complexes obtained with PGA(50)-b-Ac-AEEA(14)-Alfa show good frozen stability at low Glu/N ratios (0.2) (FIG. 47). Moreover, a stabilizing effect can be identified in the case of PGA(50)-b-pSar(50)-Alfa polymer: the presence of the Alfa-tag on the anion polymer improves stability of negatively charged particles (high Glu/N ratios) (FIG. 47) compared to anion polymer that does not bear the Alfa-tag (see FIG. 41).

### Example 17

Evaluation of Block-Copolymer of Poly-α-L-Glutamic Acid-Block-Poly-N—Ac-AEEA Length to Form Ternary Complexes with saRNA-PEI

The formation of ternary formulations of saRNA were produced after addition of a block-copolymer of Poly-α-L-glutamic acid-block-poly-N—Ac-AEEA (PGA(50)-Ac-AEEA(X), with X=4, 8, 14 repeating units).

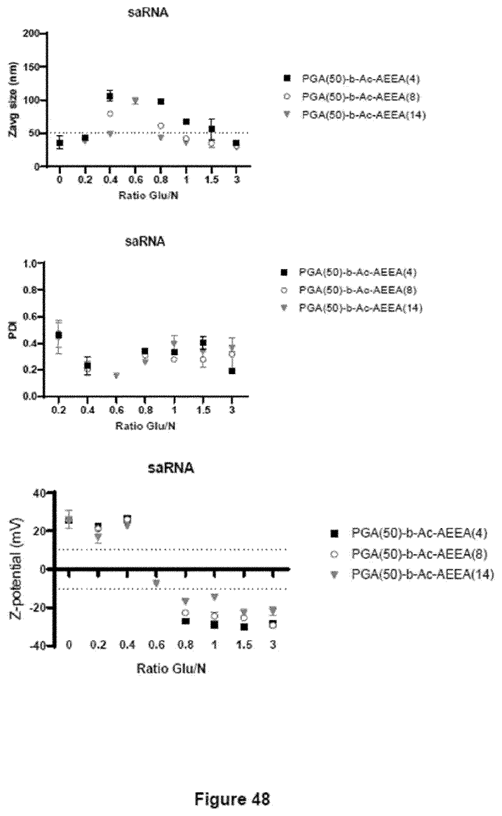

Physicochemical characterization showed ternary complexes were formed with PGA(50)-Ac-AEEA(X) with all the AEEA tested, and that the longer AEEA (PGA(50)-Ac-AEEA(14)) showed the smallest complexes sizes, Zavg≤100 nm (FIG. 48, top graph). Complex distribution was homogenous (PDI≤0.4) and surface charges of the particles change as a function of the amount of coating polymer added (zeta potential shifting from +25 to −25 mV) (FIG. 48, middle and bottom graphs).

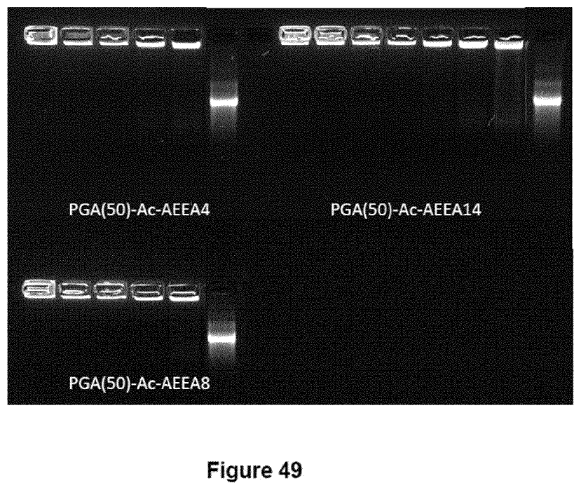

AGE reveals good saRNA encapsulation up to Glu/N of 1, with no significant effect due to stealth moiety length (FIG. 49).

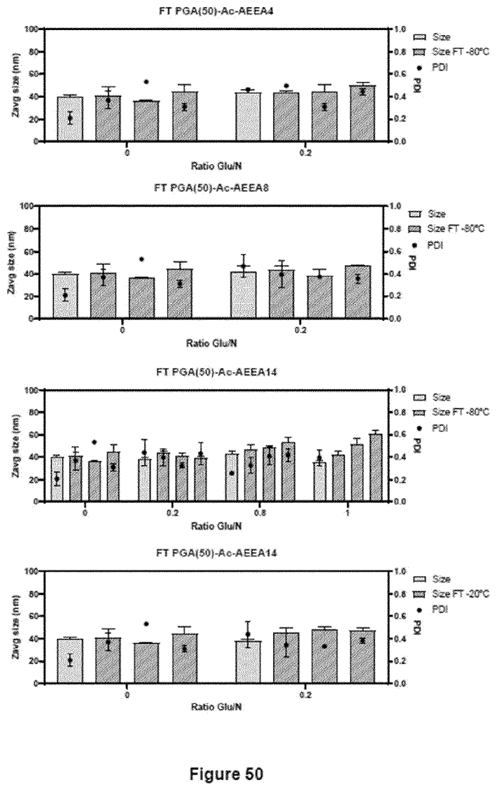

FIG. 50 showed that PGA(50)-Ac-AEEA(X) coating can guarantee colloidal stability upon freeze thawing at both −20° C. and −80° C. within certain Glu/N ratios and that PGA(50)-b-Ac-AEEA(14) is particularly efficient in maintaining colloidal properties upon repeated freeze thawing in the widest range of Glu/N tested.

The embodiments of the disclosure described above are intended to be merely exemplary, numerous variations and modifications will be apparent to those skilled in the art. All such variations and modifications are intended to be within the scope of the present invention as defined in any appended claims.

1. A complex comprising a linear cationic polymer, an anionic polymer, and a monomeric RNA molecule, wherein the cationic polymer and the monomeric RNA molecule form a core complex encapsulated by the anionic polymer.

2. The complex of claim 1, wherein the cationic polymer is or comprises copolymers of one or more of poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

3. The complex of claim 1 or 2, wherein the cationic polymer is a homopolymer selected from poly(ethylenimine), poly(propylenimine), polybrene, polyallylamine, polyvinylamine, polyamidoamine, poly-L-lysine, poly-L-arginine, poly-L-histidine, and poly(2-aminoethyl methacrylate), or a pharmaceutically acceptable salt thereof.

4. The complex of claim 2 or 3, wherein the cationic polymer is poly(ethylenimine).

5. The complex of claim 4, wherein the linear poly(ethylenimine) has a Mₙ of about 600 Da to about 400,000 Da.

6. The complex of claim 5, wherein the linear poly(ethylenimine) has a Mₙ of about 10,000 Da to about 120,000 Da.

7. The complex of any one of claim 1 or 2, wherein the cationic polymer is a block-co-polymer comprising poly(ethylenimine) and poly(propylenimine).

8. The complex of claim 1, wherein the cationic polymer is a polymer or copolymer comprising blocks of formula I and/or II: 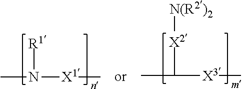
or a pharmaceutically acceptable salt thereof, wherein:
R¹ is H, —C(O)-optionally substituted C₁-C₆ aliphatic, optionally substituted C₁-C₆ aliphatic or G;
each R² is independently H, optionally substituted C₁-C₆ aliphatic, or G;
X¹ and X³ are each independently C1-C₆ aliphatic;
X² is a bond or optionally substituted C₁-C₆ aliphatic;
m is an integer between 2 and 2000;
n is an integer between 2 and 2000;
each G is independently: 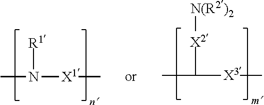
R^{1′} is H, —C(O)-optionally substituted C₁-C₆ aliphatic, optionally substituted C₁-C₆ aliphatic, or G′;
each R^{2′} is independently H, optionally substituted C₁-C₆ aliphatic, or G′;
X^{1′} and X^{3′} are each independently optionally substituted C₁-C₆ aliphatic;
X^{2′} is a bond or optionally substituted C₁-C₆ aliphatic;
n′ is an integer between 2 and 2000;
m′ is an integer between 2 and 2000;
each G′ is independently: 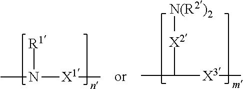
R^{1″} is H, —C(O)-optionally substituted C₁-C₆ aliphatic, or optionally substituted C₁-C₆ aliphatic;
each R^{2″} is independently H or C1-C₆ aliphatic;
X^{1″} and X^{3″} are each independently optionally substituted C₁-C₆ aliphatic;
X^{2″} is a bond or optionally substituted C₁-C₆ aliphatic;
n″ is an integer between 2 and 2000; and
m″ is an integer between 2 and 2000.

9. The complex of any one of claims 1-8, wherein the anionic polymer is a homopolymer.

10. The complex of any one of claims 1-9, wherein the anionic polymer is a homopolymer selected from poly-L-glutamic acid, poly-L-aspartic acid, a polysaccharide, poly(2-methoxycarbonylethyl-2-oxazoline), poly(2-methoxycarbonylpropyl-2-oxazoline), poly(2-methoxycarbonylethyl-2-oxazine), poly(2-methoxycarbonylpropyl-2-oxazine), and polyphosphate.

11. The complex of any one of claim 9 or 10, wherein the anionic polymer comprises from about 10 to about 100 repeating monomeric units.

12. The complex of any one of claims 1-8, wherein the anionic polymer is a heteropolymer.

13. The complex of claim 12, wherein the heteropolymer comprises monomers of glutamic acid, aspartic acid, ethylene glycol, propylene glycol, sarcosine, phosphates, 2-methyl-2-oxazoline, 2-methoxycarbonylethyl-2-oxazoline, 2-ethyl-2-oxazoline, 2-methyl-2-oxazine, 2-methoxycarbonylethyl-2-oxazine, 2-methoxycarbonylpropyl-2-oxazoline, 2-methoxycarbonylpropyl-2-oxazine, 2-(2-(2-aminoethoxy)ethoxy)acetic acid, and/or 2-(2-(2-methylaminoethoxy)ethoxy)acetic acid.

14. The complex of claim 12 or 13, wherein the anionic polymer comprises about 10 to about 100 monomeric units.

15. The complex of any one of claims 1-8, wherein the anionic polymer is a block-co-polymer.

16. The complex of claim 15, wherein the block-co-polymer comprises blocks of polymers selected from the group consisting of poly(2-methoxycarbonylethyl-2-oxazoline), poly(2-methyl-2-oxazoline), poly-L-glutamic acid, poly-L-aspartic acid, polyphosphates, poly(N-methyl-sarcosine), poly(ethylene glycol), poly(propylene glycol), poly(2-methoxycarbonylpropyl-2-oxazoline), poly(2-methoxycarbonylethyl-2-oxazine), poly(2-methoxycarbonylpropyl-2-oxazine), poly-2-(2-(2-aminoethoxy)ethoxy)acetic acid, and poly-2-(2-(2-methylaminoethoxy)ethoxy)acetic acid.

17. The complex of claim 16, wherein the block-co-polymer comprises an anionic block and a stealth block.

18. The complex of claim 17, wherein the anionic block is selected from poly(2-methoxycarbonylethyl-2-oxazoline), poly-L-glutamic acid, poly-L-aspartic acid, polyphosphate, poly(2-methoxycarbonylpropyl-2-oxazoline), poly(2-methoxycarbonylethyl-2-oxazine), and poly(2-methoxycarbonylpropyl-2-oxazoline).

19. The complex of claim 17 or 18, wherein the stealth block is selected from the group consisting of poly(ethylene glycol), poly(propylene glycol), poly(2-methyl-2-oxazoline), poly(2-ethyl-2-oxazoline), poly(2-methyl-2-oxazine), poly(N-methyl-sarcosine), poly-2-(2-(2-aminoethoxy)ethoxy)acetic acid, and poly-2-(2-(2-methylaminoethoxy)ethoxy)acetic acid.

20. The complex of any one of claims 15-19, wherein each block comprises about 10 to about 100 repeating monomeric units.

21. The complex of claim 1, wherein the cationic polymer is a linear poly(ethyleneimine), and the anionic polymer is a block co-polymer comprising block selected from poly-L-glutamic acid, poly-L-aspartic acid, and poly-2-(2-(2-aminoethoxy)ethoxy)acetic acid.

22. The complex of any one of claims 1-21, wherein the anionic polymer comprises a targeting moiety.

23. The complex of claim 22, wherein the targeting moiety is an Alfa peptide.

24. The complex of any one of claims 1-23, wherein a charge ratio of anionic group in the anionic polymer to cationic groups in the cationic polymer is from about 0.25:1 to about 3:1.

25. The complex of claim 24, wherein the charge ratio of anionic group in the anionic polymer to cationic groups in the cationic polymer is about 1.5:1.

26. The complex of any one of claims 1-25, further comprising a buffering agent.

27. The complex of claim 26, wherein the buffering agent is selected from the group consisting of 4-(2-hydroxyethyl)-1-piperazineethanesulfonic acid (HEPES), 3-morpholino-2-hydroxypropanesulfonic acid (MOPSO), 2-(N-morpholino)ethanesulfonic acid (MES), 2-Amino-2(hydroxymethyl)propane-1,3-diol (TRIS), Bis-tris buffering systems, carboxylic acid buffering systems, phosphatic acid buffering systems, and citric acid buffering systems.

28. The complex of any one of claims 1-27, wherein the RNA is mRNA, modRNA, saRNA, or taRNA.

29. The complex of any one of claims 1-28, wherein the complex has a diameter of about 10 nm to about 150 nm.

30. The complex of any one of claims 1-29, wherein the complex has a diameter of about 20 nm to about 50 nm.

31. A method of increasing or causing increased expression of RNA in a target in a subject comprising administering to the subject the complex of any one of claims 1-30.

32. The method of claim 31, wherein the target is selected from the lungs, liver, spleen, heart, brain, lymph nodes, bladder, kidneys, and pancreas.

33. A method of treating a disease, disorder, or condition in a subject comprising administering to the subject a complex of any one of claims 1-30.

34. The method of claim 33, wherein the disease, disorder, or condition is an infectious disease, cancer, a genetic disorder, an autoimmune disease, or a rare disease.

35. The method of any one of claims 31-34, wherein the complex is administered parenterally.

36. The method of claim 35, wherein the complex is administered intramuscularly, subcutaneously, or intravenously.

37. The method of any one of claims 31-34, wherein the complex is administered intranasally.

38. The method of any one of claims 31-34, wherein a first dose of the complex is administered by a first route, and a second dose of the complex is administered by a second route.

39. The method of claim 38, wherein the first route is parenteral.

40. The method of claim 38 or 39, wherein the second route is intranasal.

41. A complex of any one of claims 1-30, for use in medicine.

42. A complex of any one of claims 1-30, for use in the treatment and/or prevention of a disease, disorder, or condition, wherein the disease, disorder, or condition is an infectious disease, cancer, a genetic disorder, an autoimmune disease, or a rare disease.


SEQUENCE LISTING (10 sequences)

SEQ ID NO: 1 - protein, 5 aa, synthetic construct
CREKA

SEQ ID NO: 2 - protein, 12 aa, synthetic construct
HTMYYHHYQHHL

SEQ ID NO: 3 - protein, 31 aa, synthetic construct
KDEPQRRSARLSAKPAPPKPEPKPKKAPAKK

SEQ ID NO: 4 - protein, 12 aa, synthetic construct
AGKGTPSLETTP

SEQ ID NO: 5 - protein, 54 aa, synthetic construct
CYSASNCYSLYSALAPROGLUTHRALALEUCYSALAARGARGCYSGLNGLNHIS

SEQ ID NO: 6 - protein, 9 aa, synthetic construct
ARGGLYASP

SEQ ID NO: 7 - protein, 5 aa, synthetic construct
KLVFF

SEQ ID NO: 8 - protein, 12 aa, synthetic construct
TGNYKALHPHNG

SEQ ID NO: 9 - protein, 12 aa, synthetic construct
QSHYRHISPAQV

SEQ ID NO: 10 - protein, 13 aa, synthetic construct
SRLEEELRRRLTE